(19) United States
(12) Patent Application Publication
(10) Pub. No.: US 2024/0409957 A1
(43) Pub. Date: Dec. 12, 2024

(54) METHODS FOR PURIFICATION OF RECOMBINANT ADENO-ASSOCIATED VIRUS VECTORS FOR GENE THERAPY

(71) Applicant: Massachusetts Institute of Technology, Cambridge, MA (US)

(72) Inventors: Vivekananda Bal, Cambridge, MA (US); Anthony J. Sinskey, Cambridge, MA (US); Paul W. Barone, Cambridge, MA (US); Richard D. Braatz, Cambridge, MA (US); Stacy L. Springs, Cambridge, MA (US)

(21) Appl. No.: 18/732,263

(22) Filed: Jun. 3, 2024

Related U.S. Application Data
(60) Provisional application No. 63/505,610, filed on Jun. 1, 2023.

(51) Int. Cl. C12N 15/86

(52) U.S. Cl. CPC C12N 15/86; C12N 2750/14122; C12N 2750/14143; C12N 2750/14151

(57) ABSTRACT

Methods of purifying full capsids of recombinant adeno-associated virus vectors (rAAVs) from a solution containing a mixture of full and empty capsids are described. Typically, methods use crystallization reagents and conditions that preferentially crystalize full rAAV capsids over empty rAAV capsids. Specifically, crystallization reagents and conditions that preferentially crystalize full rAAV5 or rAAV8 capsids over their corresponding empty rAAV capsids are described. The resulting crystals containing mostly or exclusively full rAAV capsids are particularly suited for use in gene therapy. Methods of storing and reconstituting the crystals of full rAAV capsids in preparation for administering to a subject in need thereof are also provided.

### CROSS-REFERENCE TO RELATED APPLICATION

The present application claims priority to and benefit of U.S. Provisional Application No. 63/505,610, filed Jun. 1, 2023, the content of which is hereby incorporated by reference in its entirety.

### STATEMENT REGARDING FEDERALLY SPONSORED RESEARCH

This invention was made with government support under 5-RO1-FD007458-02 awarded by the US Food and Drug Administration. The government has certain rights in the invention.

### FIELD OF THE INVENTION

The invention is generally in the field of preparing and purifying adeno-associated viruses (AAVs), and more specifically using crystallization for isolating AAVs carrying recombinant DNAs for use in gene therapies.

### BACKGROUND OF THE INVENTION

Gene therapy has shown promise for the treatment of congenital blindness (Leber's congenital amaurosis), hemophilia A, hemophilia B, aromatic amino acid deficiency, and spinal muscular atrophy.¹ More than 70% of the gene therapies currently approved by drug regulatory agencies globally are viral vector based.²⁻⁴ The largest class of these gene therapies are based on recombinant adeno-associated virus vectors (rAAVs), which are the focus of more than 200 clinical trials.3 The popularity of rAAVs is due to their lack of pathogenicity, low immunogenicity, high transduction efficiency, ability to establish long-term transgene expression, lack of off-target genomic alternation, and unique property of stimulating endogenous homologous recombination (HR)-mediated double-strand DNA break repair in the genome.⁵ Gene therapy provides a means to treat many previously untreatable diseases. In gene therapy applications, the AAV genes are replaced by recombinant DNA containing therapeutic genes suitable for treating monogenic disorders. The single-stranded virion genome retains only the terminal palindromes (inverted terminal repeats or ITRs) required for vector replication and encapsidation.

Depending on the manufacturing platform, the produced rAAV particles may contain 50% to 90% empty capsids with no therapeutic transgene.⁶ The empty capsids, aka virus-like particles (VLPs), provide no therapeutic benefit, increase the capsid antigen load, reduce the transduction efficiency, and contribute to immune responses such as the recently observed thrombotic microangiopathies.⁶⁻⁹ These observations have stimulated the development of methods for removing empty capsids to improve the efficacy and safety of the therapy. However, separation of empty capsids from full capsids is extremely challenging due to very similar physical properties^{10,11} such as size, density, and isoelectric point. Chromatography and ultracentrifugation are widely used to purify or enrich for full particles,^{6,12,13} but the high cost, long processing times, additional steps, and product losses incurred make these methods undesirable for use in large-scale clinical grade vector production.¹⁴⁻¹⁶

It is an object of the invention to provide improved methods for isolating and purifying full rAAVs containing recombinant therapeutic genes suitable for gene therapies.

It is another object of the invention to provide improved methods for isolating full rAAV capsids from a mixture containing both full rAAV capsids and empty rAAV capsids.

It is yet another object of the invention to provide improved methods for isolating rAAV5, rAAV8, and rAAV9 containing recombinant therapeutic genes suitable for gene therapies.

### SUMMARY OF THE INVENTION

It has been established that under a range of crystallization conditions, full capsids of recombinant adeno-associated virus vectors (rAA Vs) containing recombinant therapeutic genes suitable for gene therapies are preferentially crystallized from a mixture that contains both full rAAV capsids and empty rAAV capsids. Thus, such preferential crystallization can be used as an effective purification step of rAAV capsids for rAAV-based gene therapies.

Accordingly, methods of purifying full rAAV capsids containing recombinant therapeutic genes from a mixture of full rAAV capsids and empty rAAV capsids having no recombinant therapeutic genes are provided. The methods typically include the steps of
(i) mixing the mixture with one or more crystallization solution, wherein the crystallization solution comprising a pre-determined set of conditions of precipitant concentration, salt concentration, and pH, wherein the pre-determined set of conditions of precipitant concentration, salt concentration, and pH favors crystallization of the full rAAV capsids over empty rAAV capsids;
(ii) providing an effective amount of time to allow crystals of the full rAAV capsids to grow; and
(iii) removing the crystals of the full rAAV capsids from the mixture, thereby purifying the full rAAV capsids from the mixture.

In some embodiments, the initial capsid concentration of the mixture of full rAAV capsids and empty rAAV capsids is between about 0.1×10¹⁴ to about 5.0×10¹⁴ vg/ml. In other embodiments, the mixture of full rAAV capsids and empty rAAV capsids contains about 20%, 30%, 40%, 50%, 60%, 70%, 80%, 90%, or more than 90% full rAAV capsids out of total full empty rAAV capsids.

In preferred embodiments, prior to step (i) the method includes a step of obtaining/finding the crystallization region (i.e., range of precipitant concentration, salt concentration, and pH over which each capsids crystallize) to provide a phase diagram (as in FIGS. 4A-4B, 5A-5B, 6A-6B, and 7A-7B) for each of the full rAAV capsids and the empty rAAV capsids by varying precipitant concentration, salt concentration, and pH; and a further step of superimposing the phase diagram for the full rAAV capsids and the phase diagram for the empty rAAV capsids (FIG. 9 was obtained by superimposing FIGS. 4B and 5B, and the FIG. 10 was obtained by superimposing FIGS. 6B and 7B) to identify the range of precipitant concentration, salt concentration, and pH that preferentially crystalizes the full rAAV capsids over empty rAAV capsids to provide the pre-determined set of conditions for preferentially crystalizing the full rAAV capsids over empty rAAV capsids. In further embodiments, the method identifies the range of precipitant concentration, salt concentration, and pH that preferentially crystalizes the empty rAAV capsids over full rAAV capsids to provide the pre-determined set of conditions for preferentially crystalizing the empty rAAV capsids over full rAAV capsids.

In other embodiments, the method includes a step of crystallizing the capsids from a mixture of full rAAV capsids and empty rAAV capsids of any composition, obtaining/finding the crystallization region (aka phase diagram) of capsids by varying the precipitant concentration, salt concentration, and pH, and then analyzing the composition of crystals obtained at different conditions to identify the range of precipitant concentration, salt concentration, and pH that preferentially crystalizes the full rAAV capsids over empty rAAV capsids to provide the pre-determined set of conditions for preferentially crystalizing the full rAAV capsids over empty rAAV capsids. In further embodiments, the method identifies the range of precipitant concentration, salt concentration, and pH that preferentially crystalizes the empty rAAV capsids over full rAAV capsids to provide the pre-determined set of conditions for preferentially crystalizing the empty rAAV capsids over full rAAV capsids.

In some embodiments, the precipitant is polyethylene glycol (PEG) such as PEG6000 and PEG8000. In the case of PEG8000, a typical concentration ranges between about 0.5 and about 8 w/v %, inclusive. In the case of PEG6000, a typical concentration ranges between about 1.0 and about 8.0 w/v %, inclusive; preferably, between about 2.0 and about 5.5 w/v %, inclusive. In some embodiments, the salt is sodium chloride, typically with a concentration ranging between about 0.01 to 2.5 M, inclusive. In some embodiments, the salt is magnesium chloride, typically with a concentration ranging between about 0.01 to 1.8 M, inclusive. In some embodiments, the pH range of the crystallization solution is between about 5.5 and about 7.5, inclusive.

The disclosed methods of purifying full rAAV capsids are generally suited for purifying rAAVs of a wide range of serotypes such as AAV1, AAV2, AAV3, AAV 3B, AAV4, AAV5, AAV6, AAV7, AAV8, AAV9, AVV10, AAV11, AAV12, BAAV, AAAV, or AAV VR-942. In preferred embodiments, the rAAVs are of serotype AAV5, AAV9, or AAV8.

In the case of serotype AAV5, the pre-determined set of conditions for full AAV5 capsids include PEG as the precipitant with a concentration between about 1.8 and about 8.0 w/v %, inclusive; sodium chloride as the salt with a concentration between about 0.3 and about 2.0 M; and a pH between about 5.5 and about 7.2. In further embodiments, the pre-determined set of conditions for full AAV5 capsids include PEG as the precipitant with a concentration between about 2.8 and about 4.3 w/v %, inclusive; sodium chloride as the salt with a concentration between about 0.8 and about 1.8 M; and a pH between about 5.6 and about 6.8.

In the case of serotype AAV9, the pre-determined set of conditions for full AAV9 capsids include PEG as the precipitant with a concentration between about 2.0 and about 6.0 w/v %, inclusive; sodium chloride as the salt with a concentration between about 0.01 and about 2 M; and a pH between about 5.5 and about 7.2. In further embodiments, the pre-determined set of conditions for full AAV9 capsids include PEG as the precipitant with a concentration between about 2.0 and about 5.0 w/v %, inclusive, preferably, between about 3 and 4.3% w/v %, inclusive; sodium chloride as the salt with a concentration between about 0.01 and about 2.0 M, preferably between about 0.35-1.65 M; and a pH between about 5.6 and about 6.9.

Typically, the methods further include a step of storing the crystals of the full rAAV capsids after purification for short-term or long-term. In some embodiments, the crystals are stored at between about 2° C. and about 20° C., preferably between about 4° C. and about 10° C. In other embodiments, one or more cryoprotectants (e.g., DMSO) are added to the crystals of the full rAAV capsids and then stored at between about −1° C. and about −80° C., preferably between about −10° C. and about −30° C. Preferably, the methods further include a step of reconstituting the crystals of the full rAAV capsids with a pharmaceutically acceptable excipient for administering to a subject in need of gene therapies. In some embodiments, the subject is in need of a genetic enzyme replacement therapy or has one or more of Duchenne muscular dystrophy, limb girdle muscular dystrophy type 2D, Leber's hereditary optic neuropathy, late infantile neuronal ceroid lipofuscinosis, rheumatoid arthritis, mucopolysaccharidosis, spinal muscular atrophy, X-linked juvenile retinoschisis, Dysferlin deficiency, hemophilia A, hemophilia B, metachromatic leukodystrophy, idiopathic Parkinson's disease, and Alzheimer's disease.

Crystals of full capsids of recombinant adeno-associated virus vectors (rAAVs) containing recombinant therapeutic genes prepared according to the disclosed methods are described. Preferably, the crystal contains minimal empty rAAV capsids having no recombinant therapeutic genes, for example, less than 10%, 9%, 8%, 7%, 6%, 5%, 4%, 3%, 2%, or 1% out of total rAAV capsids. Specifically, crystals of full capsids of recombinant adeno-associated virus vectors (rAAVs) of serotype AAV5 containing recombinant therapeutic genes prepared according to the disclosed methods are also described. In some embodiments, the methods for preparing crystals of full AAV5 capsids include PEG as the precipitant with a concentration between about 2.0 and about 8 w/v %, inclusive; sodium chloride as the salt with a concentration between about 0.35 and about 1.8 M; and a pH between about 5.5 and about 7.2. In preferred embodiments, the methods for preparing crystals of full AAV5 capsids include PEG as the precipitant with a concentration between about 2.8 and about 4.3 w/v %, inclusive; sodium chloride as the salt with a concentration between about 0.83 and about 1.75 M; and a pH between about 5.6 and about 6.8. Additionally, crystals of full capsids of recombinant adeno-associated virus vectors (rAAVs) of serotype AAV8 containing recombinant therapeutic genes prepared according to the disclosed methods are also described. In some embodiments, the methods for preparing crystals of full AAV8 capsids include PEG as the precipitant with a concentration between about 2.0 and about 4.0 w/v %, inclusive; sodium chloride as the salt with a concentration between about 0.01 and about 1.5 M; and a pH between about 5.5 and about 7.2. In preferred embodiments, the methods for preparing crystals of full AAV5 capsids include PEG as the precipitant with a concentration between about 2.0 and about 2.6 w/v %, inclusive; sodium chloride as the salt with a concentration between about 0.1 and about 1.2 M; and a pH between about 5.6 and about 6.9. Crystals of full rAAV5 or rAAV8 capsids are typically removed from the mixture solution, and optionally stored for short-term or long-term.

Methods of purifying empty rAAV capsids having no recombinant therapeutic genes from a mixture of full rAAV capsids with recombinant therapeutic genes and empty rAAV capsids are also provided.

Kits including crystallization reagents suitable for preferentially crystallizing full rAAV capsids of a particular serotype over corresponding empty rAAV capsids are also provided.

Generally, the kits include one or more crystallization solutions and instructions, optionally plates suitable for crystallization set-up. The reagents can be packaged in a single container or separate containers.

### BRIEF DESCRIPTION OF THE DRAWINGS

FIG. 1 is a schematic showing hanging-drop vapor diffusion experimental setup.

FIG. 2 shows crystallization/precipitation conditions for rAAV5 capsids as a function of initial PEG8000 concentration in droplets for NaCl concentration of 1 M at pH 7.4. The experimental results for full and empty capsids are within the error bars.

FIG. 3 is a line graph showing the effect of PEG8000 concentration and pH (6.01 and 6.17) on the content ratio at 0.6 M NaCl for the “empty” rAAV5 sample (with an initial capsid concentration of 10¹⁴ vg/ml).

FIGS. 4A-4B are phase diagram for crystallization of “full” rAAV5 at pH 5.7 (FIG. 4A) and crystallization conditions for “full” rAAV5 capsids as a function of initial NaCl and PEG concentrations and pH (FIG. 4B). The initial capsid concentration is 10¹⁴ vg/ml.

FIGS. 5A-5B are phase diagram for crystallization of “empty” rAAV5 at pH 5.7 (FIG. 5A) and crystallization conditions for “empty” rAAV5 capsids as a function of initial NaCl and PEG concentrations and pH (FIG. 5B). The initial capsid concentration is 10¹⁴ vg/ml.

FIGS. 6A-6B are phase diagram for crystallization of “full” rAAV9 capsids at pH 5.7 (FIG. 6A) and crystallization conditions for “full” rAAV9 capsids as a function of initial NaCl and PEG concentrations and pH (FIG. 6B). The initial capsid concentration is 10¹⁴ vg/ml.

FIGS. 7A-7B are phase diagrams for crystallization of “empty” rAAV9 at pH 5.7 (FIG. 7A) and crystallization conditions for “empty” rAAV9 capsids as a function of initial NaCl and PEG concentrations and pH (FIG. 7B). The initial capsid concentration is 10¹⁴ vg/ml.

FIGS. 8A-8M are sample microscopic images of the droplets, showing (FIG. 8A) dispersed phase (2.3% PEG, 0.2 M NaCl, pH 5.7, “full” AAV5) and (FIG. 8B) conversion of gel spots into crystals (2.1% PEG, 0.6 M NaCl, pH 5.7, “full” AAV5) under normal light; “empty” AAV5 crystals (1.5% PEG, 0.125 M NaCl, pH 5.7) under (FIG. 8C) normal and (FIG. 8D) polarized light; “full” AAV5 crystals (2% PEG, 0.75 M NaCl, pH 6.1) under (FIG. 8E) normal and (FIG. 8F) polarized light; “empty” AAV9 crystals (3% PEG, 0.3 M NaCl, pH 6.3) under (FIG. 8G) normal and (FIG. 8H) polarized light; “full” AAV8 crystals (3% PEG, 0.1 M NaCl, pH 6.3) under (FIG. 8I) normal and (FIG. 8J) polarized light; clear solution (FIG. 8K; AAV5, 1.5% PEG, 1.5 M NaCl), precipitate (FIG. 8L; AAV5, 4.5% PEG, 0.5 M NaCl), and (FIG. 8M; “empty” AAV5, 3% PEG, 0.5 M NaCl, pH 6.3).

FIG. 9 is a line graph showing crystallization regions for assessing the preferential crystallization of “full” and “empty” rAAV5 capsids (from FIGS. 4B and 5B). The initial capsid concentration is 10¹⁴ vg/ml.

FIG. 10 is a line graph showing crystallization regions for assessing the preferential crystallization of “full” and “empty” rAAV8 capsids (from FIGS. 6B and 7B). The initial capsid concentration is 10¹⁴ vg/ml.

FIG. 11 is a phase diagram showing crystallization regions for assessing the preferential crystallization of “full” and “empty” rAAV5 capsids at pH 5.7 and in presence of PEG6000 as a precipitant. The initial capsid concentration is 10¹⁴ vg/ml.

FIG. 12 is a phase diagram showing crystallization regions for assessing the preferential crystallization of “full” and “empty” rAAV5 capsids at pH 5.7 and in presence of MgCl2 as a salt. The initial capsid concentration is 10¹⁴ vg/ml.

FIGS. 13A-13I show experimental result for enrichment of capsids after preferential crystallization. Experimental conditions are represented by the experimental data points and belong to the preferential crystallization region of full rAAV5 capsids (FIGS. 13A-13C), empty rAAV5 capsids (FIGS. 13D-13F), and full rAAV9 capsids (FIGS. 13G-131). The corresponding error bars are the error bars from the three repetitions of the preferential crystallization experiments. Numerical value associated with each data point is the average of the values obtained from the three runs of ddPCR+ELISA of the three crystal samples obtained from three repetitions of the preferential crystallization experiments. The initial capsid concentration is 10¹⁴ vg/ml.

FIGS. 14A-14L TEM image of negative stained sample of rAAV. Row1: (FIG. 14A) reference “full” rAAV5, (FIG. 14B) reference “empty” rAAV5, (FIG. 14C) reference “full” rAAV9, and (FIG. 14D) reference “empty” rAAV9; Row2: (FIG. 14E) rAAV5 sample with ˜ 20% full capsids before crystallization, (FIG. 14F) rAAV5 stock solution after full capsid's preferential crystallization from sample (FIG. 14E), (FIG. 14G) rAAV5 sample with ˜ 20% empty capsids before crystallization, (FIG. 14H) rAAV5 stock solution after empty capsid's preferential crystallization from sample (FIG. 14G); Row3: (FIG. 14I) rAAV9 sample with ˜ 20% full capsids before crystallization, (FIG. 14J) rAAV9 stock solution after full capsid's preferential crystallization from sample (FIG. 14I), (FIG. 14K) rAAV9 sample with ˜ 20% empty capsids before crystallization, (FIG. 14L) rAAV5 stock solution after empty capsid's preferential crystallization from sample (FIG. 14K). Experiment condition: Row2: 1.3M, 3% PEG at pH 5.7 for full capsids preferential crystallization (FIG. 14F), and 0.2M, 2% PEG at pH 5.7 for empty capsids preferential crystallization (FIG. 14H); Row3: 4% PEG, 1M NaCl at pH 5.7 for full capsids preferential crystallization (FIG. 14J), and 2.75% PEG, 0.5M NaCl at pH 5.7 for empty capsids preferential crystallization (FIG. 14L).

FIGS. 15A-15D Mass photometry results obtained from Refeyn SamuxMP instrument. Percentage of different capsids for rAAV5 ((FIG. 15A) before crystallization and (FIG. 15B) after full capsids preferential crystallization) and rAAV9 ((FIG. 15C) before crystallization and (FIG. 15D) preferential crystallization of full capsids). Experiment condition: 4.2% PEG, 1.2M NaCl at pH 5.7 for rAAV5 and 3.75% PEG, 1.2M NaCl at 5.7 for rAAV9.

FIGS. 16A-16B show cell viability and fraction of GFP positive cells measured in flow cytometry experiment for full rAAV5 (FIG. 16A) and for full rAAV9 (FIG. 16B). Infection experiment was performed with MOIs: 10⁶, 5×10⁵, 10⁵, and 10⁴ for standard and crystal stock solution as indicated in the figure. Crystals were obtained at 1.4 M NaCl and 3.5% PEG at pH 6.1 for rAAV5, and at 1.5M NaCl and 4% PEG at pH 5.7 for rAAV9.

FIGS. 17A-17C show silver-stained SDS-PAGE gel electrophoresis results for rAAV5 (FIG. 17A) and rAAV9 (FIG. 17B), and western blot results for rAAV5 and rAAV9 (FIG. 17C).

FIGS. 18A and 18B: Images showing the regions of EDAX analysis on crystals for full rAAV5 (FIG. 18A) and full rAAV9 (FIG. 18B).

FIGS. 19A-19F: Microscopic image of nucleating and growing crystals for rAAV5 at different times, (FIG. 19D-19F). Experimental condition: 1.4M NaCl, 4% PEG at pH 7.2.

FIGS. 20A-20B: Yield/recovery of preferential crystallization process for full rAAV5 (FIG. 20A) and full rAAV9 (FIG. 20B)

FIGS. 21A-21B show experimental setup (FIG. 21A); and result (FIG. 21B) for preferential crystallization of full rAAV5. Experiment condition: 1.2M NaCl, 3.5% PEG at pH 5.7 Concentration of rAAV5: 5×10¹² vg/mL (2.5×10¹³ capsids or 0.25 mg capsid/batch; set 1) and 1×10¹³ vg/mL (7×10¹³ capsids or 0.7 mg capsid/batch; set 2). Batch volume: 5 mL (set 1) and 7 mL (set2).

FIG. 22: Comparison of solubilities of NaCl, PEG, and rAAV with their concentrations used in crystallization process.

FIGS. 23A-23B: Phase diagram showing the regions favorable for crystallization, precipitate formation, and undersaturated solution at pH 5.7 for “full” rAAV5 (FIG. 23A), and “empty” rAAV5 (FIG. 23B) sample.

FIGS. 24A-24D: TEM images of capsids obtained after preferential crystallization of empty rAAV5 (FIG. 24A), empty rAAV9 (FIG. 24B), full rAAV5 (FIG. 24C), and full rAAV9 (FIG. 24D). Experiment condition: for rAAV5: 0.2M, 2% PEG at pH 5.7 for empty capsids preferential crystallization (FIG. 24A), and 1.3M, 3% PEG at pH 5.7 for full capsids preferential crystallization (FIG. 24A); for rAAV9: 2.75% PEG, 0.5M NaCl at pH 5.7 for empty capsids preferential crystallization (FIG. 24B), and 4% PEG, 1M NaCl at pH 5.7 for full capsids preferential crystallization (FIG. 25D). Starting composition of capsids: 20% empty and 80% full capsids for preferential crystallization of empty rAAV5 capsids (FIG. 24A), 25% empty and 75% full capsids for preferential crystallization of empty rAAV9 capsids (FIG. 24B), 21% full and 80% empty capsids for preferential crystallization of full rAAV5 capsids (FIG. 24C), and 25% full and 75% empty capsids for preferential crystallization of full rAAV9 capsids.

FIGS. 25A-25D: SEM image of crystal surfaces obtained after preferential crystallization. Row 1 (FIG. 25A): SEM image of crystals of “empty” rAAV5 (left) and corresponding SEM image of crystal surface at higher magnification (right); 1.5% PEG, 0.4 M NaCl, pH 5.7. Row 2 (FIG. 25B): SEM image of crystals of “full” rAAV5 (left) and corresponding SEM image of crystal surface at higher magnification (right); 3% PEG, 1.6 M NaCl, pH 5.7. Row 3 (FIG. 25C): SEM image of crystals of “empty” rAAV9 (left) and corresponding SEM image of crystal surface at higher magnification (right); 2.75% PEG, 1 M NaCl, pH 5.7. Row 4 (FIG. 25D): SEM image of crystals of “full” rAAV9 (left) and corresponding SEM image of crystal surface at higher magnification (right); 3.5% PEG, 0.5 M NaCl, pH 5.7.

FIGS. 26A-26C: Crystallization conditions for “full” rAAV8 (FIG. 26A) and “empty” rAAV8 capsids (FIG. 26B) as a function of initial NaCl and PEG concentrations and pH. (FIG. 26C) shows the crystallization regions for assessing the preferential crystallization of “full” and “empty” rAAV8 capsids. The initial capsid concentration is 10¹⁴ vg/ml.

### DETAILED DESCRIPTION OF THE INVENTION

The disclosed methods are based at least on the identification of solution conditions for the preferential crystallization of full capsids. Studies characterized the effects of pH and precipitants polyethylene glycol (PEG) and salts such as sodium chloride (NaCl) and magnesium chloride (MgCl2) on the crystallization of full and empty rAAV capsids of the AAV5, AAV8, and AAV9 serotypes. The Examples demonstrate that full capsids can be selectively crystallized from a mixture of full and empty capsids for both serotypes. The data shows that preferential crystallization of full rAAV capsids can be used as a scalable and inexpensive method for rAAV capsid purification.

By contrast, the objective of prior studies on the crystallization of AAV capsids was for studying the crystallographic structure of the capsid proteins (Xie, et al., Proc Natl Acad Sci U S A 99, 10405-10410 (2002); Lerch, et al., Acta Crystallogr Sect F Struct Biol Cryst Commun 65, 177-183 (2009); Xie, Q. et al., J Virol Methods 122, 17-27 (2004); Xie, Q. et al., Acta Crystallogr Sect F Struct Biol Cryst Commun 64, 1074-1078 (2008); Miller, et al., Acta Crystallogr Sect F Struct Biol Cryst Commun 62, 1271-1274 (2006); Nam, et al., J Virol 81, 12260-12271 (2007); Mitchell, et al., Acta Crystallogr Sect F Struct Biol Cryst Commun 65, 715-718 (2009); Nam, et al., J Virol 85, 11791-11799 (2011); Mikals, et al., 186, 308-317 (2014); Kaludov, et al., Virology 306, 1-6 (2003)). Thus, prior studies crystallized either full or empty AAV capsids from aqueous solution of purified sample obtained after chromatography and/or ultracentrifugation purification for each of the serotypes AAV1 through AAV9, except for AAV5 and AAV7. The crystals were used in x-ray diffraction pattern analysis to determine the crystal structure orientation and number of capsids per unit cell (the unit cell is composed of a single capsid in most cases, and of 3 capsids in some cases, e.g., AAV9), and the surface features of the capsids with an aim to contribute to the ongoing effort to develop vectors for tissue-targeted gene therapy. In addition, efforts were made to provide information on the capsid surface regions, which can be modified to generate vectors that can evade the pre-existing antibody responses against the capsid for improved therapeutic efficiency. However, these prior studies did not consider the possibility of preferential crystallization of either full or empty capsids as a method for the industrial-scale purification of AAVs.

### I. Definitions

The term “crystal” refers to a solid material, whose constituent atoms, molecules, or ions are arranged in an orderly repeating pattern extending in all three spatial dimensions. The process of forming a crystalline structure from a fluid or from materials dissolved in the fluid is often referred to as “crystallization”. Protein (or rAAV) crystals are almost always grown in solution. The most common approach is to lower the solubility of its component molecules gradually. Crystal growth in solution is characterized by two steps: nucleation of a microscopic crystallite, followed by growth of that crystallite.

The terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

The term “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The term “pharmaceutically acceptable salt”, as used herein, refers to derivatives of the compounds defined herein, wherein the parent compound is modified by making acid or base salts thereof. Lists of suitable salts are found in Remington's Pharmaceutical Sciences, 20th ed., Lippincott Williams & Wilkins, Baltimore, MD, 2000, p. 704; and “Handbook of Pharmaceutical Salts: Properties, Selection, and Use,” P. Heinrich Stahl and Camille G. Wermuth, Eds., Wiley-VCH, Weinheim, 2002.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%: in other embodiments the values may range in value either above or below the stated value in a range of approx. +/−5%: in other embodiments the values may range in value either above or below the stated value in a range of approx. +/−2%: in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound or method is disclosed and discussed and a number of modifications that can be made to a number compositions, methods, systems, etc. including the compound are discussed, each and every combination and permutation of compound or method and the modifications that are possible are specifically contemplated unless specifically indicated to the contrary. Thus, for example, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. For example, same rationale and corresponding disclose also applies to methods and method steps as is discussed for molecules. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Compositions of Crystals of Full rAAV Capsids

Crystals containing full capsids of recombinant adeno-associated virus vectors (rAAVs) are described. In preferred embodiments, the crystals contain mostly or exclusively full rAAV capsids, for example more than 90%, 91%, 92%, 93%, 94%, 95%, 96%, 97%, 98%, or 99% out of the total rAAV capsids within the crystals. In a particular embodiment, the crystals contain greater than 95% full rAAV capsids out of the total rAAV capsids within the crystals.

In some embodiments, crystals are mostly anisotropic, cylindrical, rod shaped, needle shaped, and some undefined shape. In some embodiments, crystals are between about 2 micrometers and about 100 micrometers in length. Generally, crystals contain small/permissible amount of salt as an impurity. Crystals dissolve instantly in the PBS buffer at a pH of 7.4 and is stable in presence of one or more excipients such as PEG.

In some embodiments, the rAAVs are of serotype of AAV1, AAV2, AAV3, AAV 3B, AAV4, AAV5, AAV6, AAV7, AAV8, AAV9, AVV10, AAV11, AAV12, BAAV, AAAV, or AAV VR-942.

In preferred embodiments, the rAAVs are serotype AAV5, AAV8, or AAV9.

In some embodiments, crystals of full capsids of recombinant adeno-associated virus vectors (rAAVs) containing recombinant therapeutic genes prepared according to the disclosed methods are described. Preferably, the crystal is essentially free of empty rAAV capsids having no recombinant therapeutic genes.

Specifically, crystals of full capsids of recombinant adeno-associated virus vectors (rAAVs) of serotype AAV5 containing recombinant therapeutic genes prepared according to the disclosed methods are also described. In some embodiments, the methods for preparing crystals of full AAV5 capsids include PEG as the precipitant with a concentration between about 1.8 and about 5.5 w/v %, inclusive; sodium chloride as the salt with a concentration between about 0.35 and about 2.0 M; and a pH between about 5.5 and about 7.2. In preferred embodiments, the methods for preparing crystals of full AAV5 capsids include PEG as the precipitant with a concentration between about 2.8 and about 4.3 w/v %, inclusive; sodium chloride as the salt with a concentration between about 0.8 and about 1.8 M; and a pH between about 5.6 and about 6.8.

Crystals of full capsids of recombinant adeno-associated virus vectors (rAAVs) of serotype AAV8 containing recombinant therapeutic genes prepared according to the disclosed methods are also described. In some embodiments, the methods for preparing crystals of full AAV8 capsids include PEG as the precipitant with a concentration between about 2.0 and about 4.5 w/v %, inclusive; sodium chloride as the salt with a concentration between about 0.1 and about 1.5 M; and a pH between about 5.0 and about 6.0. In preferred embodiments, the methods for preparing crystals of full AAV8 capsids include PEG as the precipitant with a concentration between about 2.0 and about 2.6 w/v %, inclusive; sodium chloride as the salt with a concentration between about 0.1 and about 1.2 M; and a pH between about 5.6 and about 6.7. Crystals of full rAAV5 or rAAV8 capsids are typically removed from the mixture solution, and optionally stored for short-term or long-term.

### III. Methods of Obtaining Crystals of Full rAAV Capsids

Methods of crystalizing full rAAV capsids are provided. Preferably, the methods can selectively crystalize full rAAV capsids containing desired recombinant therapeutic genes suitable for gene therapies from a mixture that contains both full rAAV capsids and empty rAAV capsids.

Methods generally include a step of (a) preparing a solution of rAAV capsids, (b) mixing the solution with a crystallization solution, and (c) allowing nucleation and growth of crystals.

In some embodiments, the initial capsid concentration of the mixture solution of full rAAV capsids and empty rAAV capsids is between about 0.1×10¹⁴ to about 5×10¹⁴ vg/ml. In other embodiments, the mixture of full rAAV capsids and empty rAAV capsids contains about 20%, 30%, 40%, 50%, 60%, 70%, 80%, 90%, or more than 90% full rAAV capsids out of total full empty rAAV capsids.

Crystallization requires bringing the rAAV capsids to supersaturation. The sample should therefore be concentrated to the highest possible concentration without causing aggregation or precipitation of the rAAV capsids. Introducing the sample to precipitating agent can promote the nucleation of protein crystals in the solution, which can result in large three-dimensional crystals growing from the solution.

Methods for crystalizing include vapor diffusion or interface diffusion. In some embodiments, the methods use vapor diffusion, for example, sitting drop vapor diffusion or hanging drop vapor diffusion. In vapor diffusion, a drop containing a mixture of precipitant and rAAV capsid solutions is sealed in a chamber holding a reservoir solution of pure precipitant. Water vapor then diffuses out of the drop until the osmolarity of the drop and the reservoir solution are equal. The dehydration of the drop causes a slow increase in concentration of both rAAV capsid and precipitant until equilibrium is achieved, ideally in the crystal nucleation zone of the phase diagram.

In a preferred embodiment, hanging drop vapor diffusion is used. in some embodiments, each droplet contains a mixture of rAAV sample and crystallization buffer solution of 1:1 ratio, e.g., 1 μL of rAAV sample and 1 μL of buffer solution. In other embodiments, the ratio can vary to optimize crystallization of full rAAV over empty rAAV.

In some embodiments, a sparse matrix and incomplete factorial screen of precipitating conditions is used prior to a fine screen of conditions, e.g., precipitants, salts, and pH. As shown in the Example, one or more initial screenings can be performed by varying the concentration of PEG 8000, and one or more subsequent screenings can be conducted by varying the salt (e.g., NaCl) concentration to fine-tune the ionic strength of the crystallization medium.

In some embodiments, the crystallization setup is automated and carried out by robotics. In an exemplary case, the automated setup contains a flow cell, which holds a droplet from the top cover slide and allows air of controlled temperature and humidity to flow through horizontally. Measurements of the humidity in the flow cell are sent to a programmable logic controller which manipulates the ratio of humid and dry air in the upstream flow to achieve a desired humidity (e.g., ˜94%). The temperature in the flow cell is controlled by a programmable circulating water bath, with typical setpoint values of ˜4° C.

In other embodiments, the crystallization setup is carried out manually.

### A. Crystallization Reagents and Methods

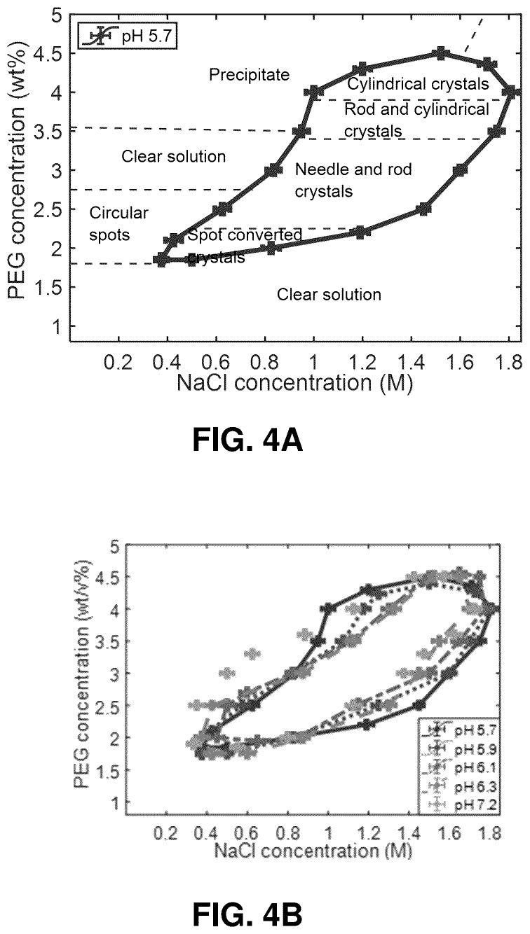

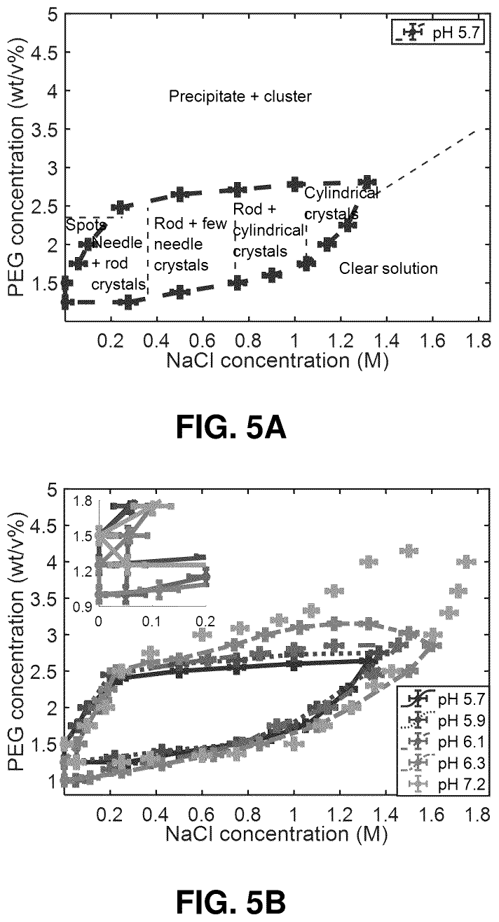

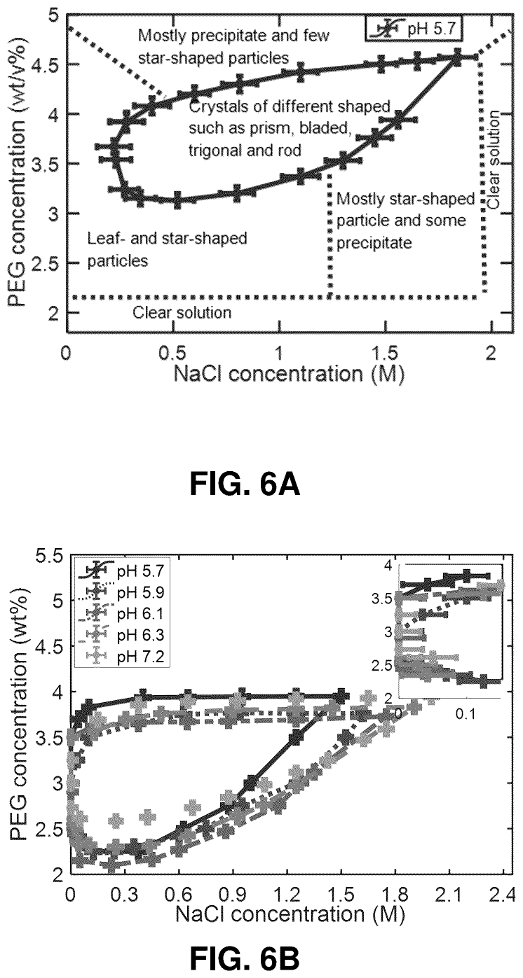

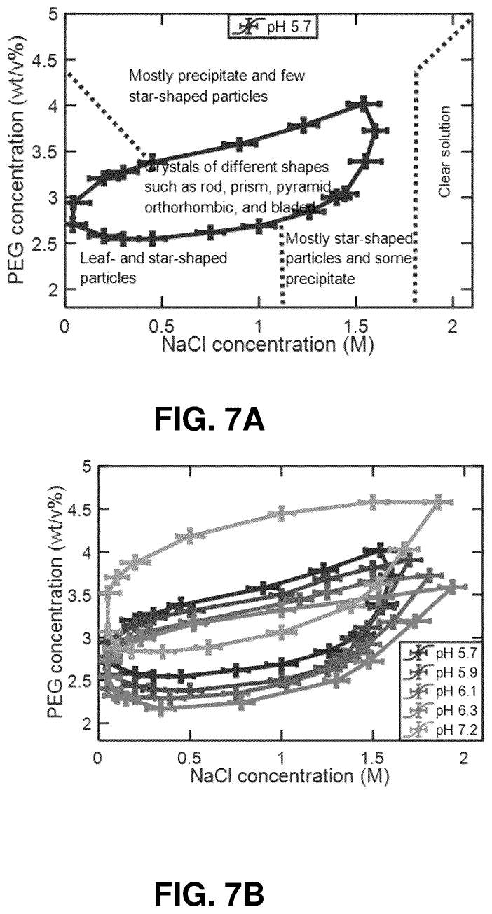

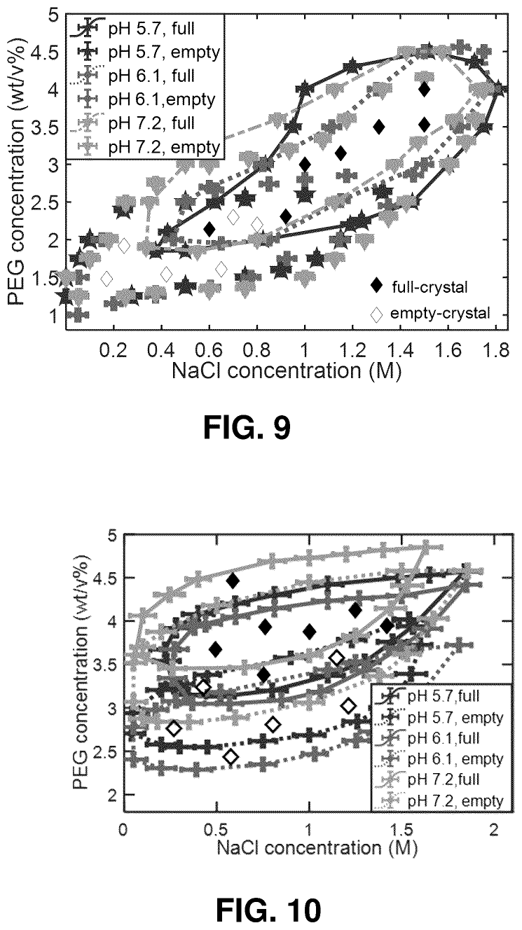

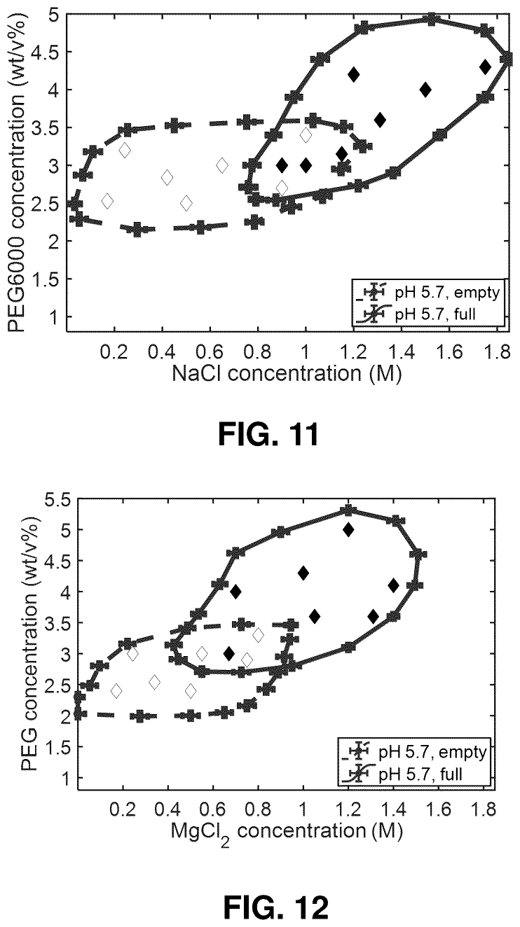

To establish crystallization conditions that preferentially crystallize full rAAV capsids from a mixture that contains both full rAAV capsids and empty rAAV capsids, typically the method includes a step of obtaining/finding the crystallization region (i.e., range of precipitant concentration, salt concentration, and pH over which each capsids crystallize) to provide a phase diagram (as in FIGS. 4A-4B, 5A-5B, 6A-6B, and 7A-7B) for each of the full rAAV capsids and the empty rAAV capsids by varying precipitant concentration, salt concentration, and pH; and a further step of superimposing the phase diagram for the full rAAV capsids and the phase diagram for the empty rAAV capsids FIG. 9 was obtained by superimposing FIGS. 4B and 5B, and the FIG. 10 was obtained by superimposing FIGS. 6B and 7B) to identify the range of precipitant concentration, salt concentration, and pH that preferentially crystalizes the full rAAV capsids over empty rAAV capsids to provide the pre-determined set of conditions for preferentially crystalizing the full rAAV capsids over empty rAAV capsids. In further embodiments, the method identifies the range of precipitant concentration, salt concentration, and pH that preferentially crystalizes the empty rAAV capsids over full rAAV capsids to provide the pre-determined set of conditions for preferentially crystalizing the empty rAAV capsids over full rAAV capsids.

In other embodiments, the method includes a step of crystallizing the capsids from a mixture of full rAAV capsids and empty rAAV5 capsids of any composition, obtaining/finding the crystallization region (aka phase diagram) of capsids by varying the precipitant concentration, salt concentration, and pH, and then analyzing the composition of crystals obtained at different conditions to identify the range of precipitant concentration, salt concentration, and pH that preferentially crystalizes the full rAAV capsids over empty rAAV capsids to provide the pre-determined set of conditions. In further embodiments, the method identifies the range of precipitant concentration, salt concentration, and pH that preferentially crystalizes the empty rAAV capsids over full rAAV capsids to provide the pre-determined set of conditions for preferentially crystalizing the empty rAAV capsids over full rAAV capsids.

In some embodiments, the precipitant is polyethylene glycol (PEG) such as PEG6000 and PEG8000. In the case of PEG8000, a typical concentration ranges between about 0.5 and about 8 w/v %, inclusive. In the case of PEG6000, a typical concentration ranges between about 1.0 and about 8.0 w/v %, inclusive; preferably, between about 2.0 and about 5.5 w/v %, inclusive. In some embodiments, the salt is sodium chloride, typically with a concentration ranging between about 0.01 to 2.5 M, inclusive. In some embodiments, the salt is magnesium chloride, typically with a concentration ranging between about 0.01 to 1.8 M, inclusive. In some embodiments, the pH range of the crystallization solution is between about 5.5 and about 7.5, inclusive.

### 1. Precipitants

Precipitants of macromolecules fall into four broad categories: (i) salts, (ii) organic solvents, (iii) long-chain polymers and (iv) low-molecular-weight polymers and nonvolatile organic compounds. The first two classes are typified by ammonium sulfate and ethyl alcohol, respectively, and higher polymers such as polyethylene glycol 4000 are characteristic of the third category. In the fourth category, exemplary compounds include methyl-pentanediol and polyethylene glycols of molecular weight lower than about 1000.

In preferred embodiments, the precipitant is polyethylene glycol (PEG). PEGs with molecular weights from 400 to 20,000 have successfully provided protein crystals, but the most useful are those in the range 2,000-8,000. The molecular weights are generally not completely interchangeable for a given protein, even within the mid-range. This is a parameter which is best optimized by empirical means along with concentration and temperature. The very low molecular weight PEGs such as PEG 200 and 400 are rather similar in character to MPD and hexanediol. There does not appear to be any correlation between the molecular weight of a protein and that of the PEG best used for its crystallization. The higher molecular weight PEGs do, however, have a proportionally greater capacity to force proteins from solution.

In preferred embodiments, the precipitant is polyethylene glycol (PEG). Exemplary PEGs suitable for macromolecular crystallization include PEG4000, PEG6000, PEG8000, and PEG10,000.

In one embodiment, the precipitant is PEG8000. Suitable concentrations of PEG8000 for crystallizing full rAAV capsids range between 0.5 to 8 wt/v %). As seen in Example, for rAAV5, the preferential crystallization for full capsids is favorable at higher PEG/NaCl concentration and at lower pH (pH<7.2), empty rAAV5 crystallization region is restricted to a low PEG and NaCl concentration. For rAAV8: low pH of 5.7 is favorable for preferential crystallization of full capsids only; preferential crystallization region for full capsids is in low PEG concentration at almost all pH except pH of 7.2.

### 2. Salts

Sodium chloride is widely used as an inorganic precipitant in the crystallization of proteins (Yamanaka, M. et al., J Synchrotron Radiat 18, 84-87 (2011); Hekmat, D. Bioprocess Biosyst Eng 38, 1209-1231 (2015)) to shift the protein solubility by the changing the number of water molecules available for interaction with the charged part of a protein's surface.

In preferred embodiments, the salt in the crystallization solution is NaCl. Suitable concentrations of NaCl for crystallizing full rAAV capsids range between 0.01 to 2.5 M.

3. pH

Along with ionic strength, pH is one of the most important variables influencing the solubility of proteins or rAAVs. This variable provides another powerful approach to creating supersaturated solutions, and thus effecting crystallization. Its manipulation at various ionic strengths and in the presence of diverse precipitants is a fundamental idea in formulating screening matrices and discovering successful crystallization conditions. Examples of the effect of pH on rAAVs is illustrated in FIGS. 4B, 5B, 6B, and 7B.

In some embodiments, the pH range of the crystallization solution for crystallizing full rAAV capsids is between about 5.5 and about 8.0, inclusive; or between about 5.5 and about 7.5, inclusive.

### 4. Seeding

In some cases, it is desirable to reproduce previously grown crystals of rAAVs. This can sometimes be accomplished by seeding a metastable, supersaturated solution of rAAVs with crystals from earlier trials.

The seeding techniques fall into two categories: those employing microcrystals as seeds and those using larger macro seeds. In both methods, the fresh solution to be seeded should be only slightly supersaturated so that controlled, slow growth will occur.

### 5. Robotics and Automation of Crystallization

In some embodiments, robotics is used to provide a large-scale crystallization of rAAVs, particularly in cases where the crystallization condition is also established and/or optimized. In one embodiment, the processing volume is about 100 mL of AAVs solution. N some forms, the processing volume is up to 1 L, 100 L, 1000 L etc.

B. Methods of Verifying Crystals of Full rAAV Capsids

In some embodiments, the methods include a step of confirming that the crystals are formed of rAAV capsids, not salt crystals.

In some embodiments, crystals are observed through normal and cross-polarized light microscopes to determine the optical properties (e.g., crystal birefringence) of the particles. NaCl salt crystals are cubic in shape and are isotropic in nature. Therefore, they will not show any birefringence and mostly remain extinct under cross polarized light. On the other hand, protein crystals are anisotropic in nature and show strong birefringence. Presence of birefringence suggests that the materials/particles formed are protein crystals and since the crystals are not cubic in shape, they are definitely from AAV capsids and are not salt crystals. Polarization experiment is the initial screening of the crystals.

In some embodiments, control experiments are performed in absence of capsids but maintaining the other conditions same to determine whether the crystals are from capsids. If no crystals are formed in the control experiments, then it suggests that crystals formed in presence of capsids are from capsids.

In other embodiments, the solubilities of different components used in the crystallization experiment are compared. The concentrations of components used in crystallization experiments remains well below their solubility limits. This suggests that crystals are from capsids only.

In some embodiments, X-ray diffraction (XRD) is used to confirm the crystallinity of the material. In other embodiments, where crystal amount is small, crystallinity is determined by selected area electron diffraction (SAED) analysis in trans-mission electron microscopy (TEM).

In preferred embodiments, the methods include a step of confirming that the crystals are formed of mostly all full rAAV capsids, not a mixture of full/empty capsids or empty capsids.

In some embodiments, percentages of full and empty capsids in the crystals are determined. Exemplary methods include quantitative PCR (e.g., DROPLET DIGITAL™ PCR) or ELISA experiments, described in detail in the Example. Stock solution of rAAV is prepared by dissolving crystals for biological assays. To prepare AAV stock solution, crystals are collected from the droplet following the same procedure in Section entitled “Cryo-TEM of AAV crystals to obtain electron diffraction pattern (SAED)”, below. The collected crystals are collected and washed according to the procedure described in Section entitled “Room temperature TEM analysis of AAV particles after crystallization”, below. The AAV crystals are dissolved by incubating in 300 μL of nuclease-free water at 4° C. refrigerator for 30 min. The dissolved crystal solution is transferred into an Amicon centrifugal filter (10 kDa NMWCO) and centrifuged at 10000 g for 5 min. Capsids are then washed 3 times using 200 μL of nuclease-free water at 10000 g for 5 min each time to ensure complete removal of remaining salt and precipitant. Harvested capsid particles are then resuspended into 200 μL of Dulbecco's 1× PBS (DPBS without magnesium and calcium) and collected into a vial and stored at 4° C. for short-term use. For long-term storage, 5% Sorbitol solution is added into the capsids suspension and stored in a cryovial at −80° C. . . .

In further embodiments, cell infection by rAAV present in crystals is carried out in cells (e.g., HEK293T cell) in vitro to test the biological activity of these rAAV.

### C. Harvest of Crystals

Generally, the crystals containing full rAAV capsids are harvested and removed from the mixture solution. In some embodiments, the crystals are harvested and stored in the same crystallization reagents from where the crystals were formed. In some embodiments, the crystals containing full rAAV capsids are washed one or more times in the crystallization reagents to further minimize any adsorbed empty rAAV capsids from the crystallization drop.

The crystals containing full rAAV capsids can be stored for short-term and long-term prior to their dissolution in a pharmaceutically acceptable carrier for use in gene therapy.

### D. Storage

Methods of preparing crystals containing full capsids of recombinant adeno-associated virus vectors (rAAVs) for short-term and long-term storage are also described. In some embodiments, the disclosed crystals containing full rAAV capsids are stored at room temperature, or between about 2° C. and about 20° C., preferably between about 4° C. and about 10° C. In preferred embodiments, storing full capsids in the form of crystals yields a good recovery after the desired period of time, with minimal loss of its biological properties.

In other embodiments, the disclosed crystals containing full rAAV capsids are subject to freezing for long-term storage between about −1° C. and about −80° C., preferably between about −10° C. and about −30° C., or about −20° C. In some cases where freezing is used, cryoprotectants can be added to the solution prior freezing to protect and further stabilize these crystals containing full rAAV capsids. Examples of cryoprotectants include sucrose, trehalose, raffinose, stachyose, verbascose, mannitol, glucose, lactose, maltose, maltotriose-heptaose, dextran, hydroxyethyl starch, sorbitol, glycerol, arginine, histidine, lysine, proline, dimethylsulfoxide, or any combination thereof. The concentration of the cryoprotectant in the formulation is in an effective amount to yield a good rate of recovery after one or more freezing-thawing cycles, for example, the concentration of the cryoprotectant ranges from about 0.05% to about 50% w/v (e.g., from about 0.05% to about 25% w/v, from about 1% to 15% w/v, from about 3% to about 12.5% w/v, from about 1% to about 8% w/v, or from about 2% to about 7% w/v).

Generally, the method provides a good recovery rate after one or more freezing-thawing cycles after a period of time (e.g., one month or one year), about 50% or more, 60% or more, 70% or more, 80% or more, 90% or more, 95% or more, or 99% or more of intact full rAAV capsids, as compared to the number of full rAAV capsids in the crystals prior to one or more freezing-thawing cycles. In preferred embodiments, after thawing from being frozen and stored with or without one or more cryoprotectants for a period of time the full rAAV capsids retain their size, content, and biological/therapeutic activity.

### IV. Methods of Isolating Full rAAV Capsids for Gene Therapy

Methods of purifying full rAAV capsids containing recombinant therapeutic genes from a mixture of full rAAV capsids and empty rAAV capsids having no recombinant therapeutic genes are provided.

As demonstrated in the Example that under a range of crystallization conditions, full capsids of recombinant adeno-associated virus vectors (rAAVs) containing recombinant therapeutic genes suitable for gene therapies are preferentially crystallized from a mixture that contains both full rAAV capsids and empty rAAV capsids. Thus, such preferential crystallization can be used as an effective purification step of rAAV capsids for rAAV-based gene therapies.

The methods typically include the steps of
(i) mixing the mixture with one or more crystallization solution, wherein the crystallization solution comprising a pre-determined set of conditions of precipitant concentration, salt concentration, and pH, wherein the pre-determined set of conditions of precipitant concentration, salt concentration, and pH favors crystallization of the full rAAV capsids over empty rAAV capsids;
(ii) providing an effective amount time to allow crystals of the full rAAV capsids to grow; and
(iii) removing the crystals of the full rAAV capsids from the mixture, thereby purifying the full rAAV capsids from the mixture.

In some embodiments, the initial capsid concentration of the mixture of full rAAV capsids and empty rAAV capsids is between about 0.1×10¹⁴ to about 5×10¹⁴ vg/ml. In other embodiments, the mixture of full rAAV capsids and empty rAAV capsids contains about 50%, 60%, 70%, 80%, 90%, or more than 90% full rAAV capsids out of total full and empty rAAV capsids.

Characterization of crystallization kinetics for a particular serotype of rAAV involves screening of crystallization conditions by varying concentrations of precipitant (e.g., PEG8000, PEG6000) and salt (e.g., NaCl, MgCl2) at different pH values to establish a crystallization profile of both full rAAV capsids and the corresponding empty rAAV capsids. An exemplary concentration range suitable for screening rAAV are 0.01 to 2.5 M for NaCl, 0.5 to 8.0 wt/v % for PEG8000, and a pH range of about 5.0 to about 8.0.

Thus, in preferred embodiments, prior to step (i) the method includes a step of obtaining/finding the crystallization region (i.e., range of precipitant concentration, salt concentration, and pH over which each capsids crystallize) to provide a phase diagram (as in FIGS. 4A-4B, 5A-5B, 6A-6B, and 7A-7B) for each of the full rAAV capsids and the empty rAAV capsids by varying precipitant concentration, salt concentration, and pH; and a further step of superimposing the phase diagram for the full rAAV capsids and the phase diagram for the empty rAAV capsids (FIG. 9 was obtained by superimposing FIGS. 4B and 5B, and the FIG. 10 was obtained by superimposing FIGS. 6B and 7B) to identify the range of precipitant concentration, salt concentration, and pH that preferentially crystalizes the full rAAV capsids over empty rAAV capsids to provide the pre-determined set of conditions. In further embodiments, the method identifies the range of precipitant concentration, salt concentration, and pH that preferentially crystalizes the empty rAAV capsids over full rAAV capsids to provide the pre-determined set of conditions.

In other embodiments, the method includes a step of crystallizing the capsids from a mixture of full rAAV capsids and empty rAAV5 capsids of any composition, obtaining/finding the crystallization region (aka phase diagram) of capsids by varying the precipitant concentration, salt concentration, and pH, and then analyzing the composition of crystals obtained at different conditions to identify the range of precipitant concentration, salt concentration, and pH that preferentially crystalizes the full rAAV capsids over empty rAAV capsids to provide the pre-determined set of conditions. In further embodiments, the method identifies the range of precipitant concentration, salt concentration, and pH that preferentially crystalizes the empty rAAV capsids over full rAAV capsids to provide the pre-determined set of conditions.

In some embodiments, the precipitant is polyethylene glycol (PEG) such PEG6000 and as PEG8000. In the case of PEG8000, a typical concentration ranges between about 0.5 and about 8 w/v %, inclusive. In the case of PEG6000, a typical concentration ranges between about 1.0 and about 8.0 w/v %, inclusive; preferably, between about 2.0 and about 5.5 w/v %, inclusive. In some embodiments, the salt is sodium chloride, typically with a concentration ranging between about 0.01 to 2.5 M, inclusive. In some embodiments, the salt is magnesium chloride, typically with a concentration ranging between about 0.01 to 1.8 M, inclusive. In some embodiments, the pH range of the crystallization solution is between about 5.5 and about 7.5, inclusive.

Typically, the methods further include a step of storing the crystals of the full rAAV capsids after purification for short-term or long-term. In some embodiments, the crystals are stored at between about 2° C. and about 20° C., preferably between about 4° C. and about 10° C. In other embodiments, one or more cryoprotectants are added to the crystals of the full rAAV capsids and then stored at between about −1° C. and about −80° C., preferably between about −10° C. and about −30° C. Preferably, the methods further include a step of reconstituting the crystals of the full rAAV capsids with a pharmaceutically acceptable excipient for administering to a subject in need of gene therapies.

Exemplary uses in gene therapies include a genetic enzyme replacement therapy or for treating one or more of Duchenne muscular dystrophy, limb girdle muscular dystrophy type 2D, Leber's hereditary optic neuropathy, late infantile neuronal ceroid lipofuscinosis, rheumatoid arthritis, mucopolysaccharidosis, spinal muscular atrophy, X-linked juvenile retinoschisis, Dysferlin deficiency, hemophilia A, hemophilia B, metachromatic leukodystrophy, idiopathic Parkinson's disease, and Alzheimer's disease.

### V. Kits

Kits including crystallization reagents suitable for preferentially crystallizing full rAAV capsids of a particular serotype over corresponding empty rAAV capsids are also provided. Generally, the kits include one or more crystallization solutions and instructions, optionally plates suitable for the crystallization set-up. The reagents can be packaged in a single container or separate containers.

The present invention will be further understood by reference to the following non-limiting examples.

### EXAMPLES

Gene therapies have been demonstrated to cure numerous diseases but have high manufacturing costs. The cells used to produce genome-loaded capsids (aka full capsids) for gene therapies also produce capsids that do not contain genomes (aka empty capsids), which activate the immune system without providing therapeutic benefit, requiring the removal of most of the empty capsids during manufacturing. The processes for separating full and empty capsids have low efficiency due to the high similarity of properties between full and empty capsids, which has motivated efforts to invent more efficient processes. The current study presents data that indicate that a mixture of full and empty capsids is separable by using solution crystallization—a purification process that is widely used in the small-molecule pharmaceutical industry due to having high yields and low capital and operating costs. Hanging-drop vapor diffusion experiments and electron diffraction (SAED) analysis are used to determine the combinations of pH and polyethylene glycol (PEG) and sodium chloride concentrations in which full and empty capsids crystallize, which define conditions in which crystals of full, empty, or both full and empty capsids nucleate and grow. Differences in crystallization behavior of full and empty capsids are related to differences in capsid-capsid interactions associated with differences in effective surface charge due to presence or absence of genomes in the capsids. The results show that full capsids can be selectively crystallized from a mixture of full and empty capsids.

### Example 1: Preferential Crystallization of rAA Vs in a Hanging Drop Crystallizer for Purification of rAAV-Based Gene Therapies

### Material and Methods

### Materials

All chemicals required for the experiments were of molecular biology grade. Chemicals purchased from Sigma-Aldrich were sodium dihydrogen phosphate dihydrate (BioUltra, ≥99.0%), sodium hydroxide (BioXtra, ≥98% anhydrous), 1M hydrochloric acid (BioReagent, for cell culture), sodium chloride (BioXtra, ≥99.5%), potassium chloride (BioXtra, ≥99.5%), potassium dihydrogen phosphate (BioUltra, ≥99.5%), magnesium chloride (BioReagent, ≥ 97%), phosphate-buffered saline (PBS 1× (150 mM sodium phosphate and 150 mM NaCl), pH 7.2; BioUltra solution), polyethylene glycol (PEG-8000, BioUltra; PEG-6000, BioUltra), Phosphotungstic acid (10% solution), and Propidium iodide solution (1 mg/mL in water). Dulbecco's 1× PBS, FreeStyle 293 F cells, FreeStyle 293 expression medium, DMEM 1× (Dulbecco's modified Eagle medium), 0.25% Trypsin-EDTA (1×), FBS (fetal bovine serum) were purchased from Thermo Fisher Scientific. Nuclease-free water (Ambion nuclease-free water), Trypan blue stain (0.4%), and countess II automated counting slides were purchased from Invitrogen. Poloxamer-188 (Pluronic F-68, 10%, BioReagent) was added to rAAV-containing solutions at 0.001% to suppress binding to container surfaces. Glutaraldehyde solution (10% aqueous solution) and copper grid (carbon support film, 200 mesh, Cu; CF200-CU-50) were purchased from Electron Microscopy Sciences. Amicon Ultra centrifugal filter (10 kDa, UFC5010) and Ultrafree MC VV centrifugal filters (PVDF 0.1 μm; UFC30VV25) were purchased from Millipore Sigma. Falcon tube (5 mL, polystyrene round-bottom tube with cell-strainer cap), cell culture dish (10 cm×2 cm), Erlenmeyer shake flask (125 mL), Costar 96-well cell culture plate (TC treated, sterile), and 6-well cell culture plate (TC treated, sterile) were purchased from Corning. An ELISA kit was purchased from Progen and stored in a 4° C. refrigerator. DNA primers were purchased from Integrated DNA Technologies (Coralville, IA, USA). Eva Green Supermix was purchased from Bio-Rad Laboratories. The cover glasses and the silicon gaskets containing six sample wells for mass photometry measurement were provided by Refeyn Ltd. (MA, USA).

rAAV Samples

Recombinant adeno-associated virus serotypes, rAAV5 and rAAV9, both as full (genome-loaded) and empty (without genome) capsids, were purchased from Virovek at a reported concentration of 10¹⁴ vg/ml. The sample labeled as “full rAAV5” (Lot 19-047E) from Virovek actually contains 80% full and 20% empty capsids, and the sample labeled as “empty rAAV5” (Lot 19-253E) from Virovek contains 92% empty and 8% full capsids. Similarly, the sample labeled as “full rAAV9” (Lot 21-100) from Virovek contains 80% full and 20% empty capsids, and the sample labeled as “empty rAAV9” (Lot 21-077) contains 96% empty and 4% full capsids. Virovek quantified the full and empty capsids using qPCR and nanodrop OD (optical density) measurement. In-house quantification of full and empty capsids using ddPCR and ELISA produced roughly similar results as reported by Virovek. The full rAAV capsids contained a genome of length 2.5 kbp with cytomegalovirus (CMV) promoter expressing green fluorescent protein (GFP). For each type of sample, Virovek supplied the sample from the same batch, so the rAAV samples in our experiments do not have the variability that can occur when samples are taken from different batches. rAAV samples were supplied at a pH of 7.2 in PBS buffer in small vials holding 100 μL samples each, and were stored long-term at −80° C. Before putting the samples into the freezer, 100 μL sample from each vial was divided into 5 equal aliquots. Aliquots are stored at −80° C. for long-term use. For short-term use, based on requirement, small vials were taken out of the −80° C. freezer and stored at 4° C., at which AAV is stable for 4 weeks.^{29,30}

### Experiment

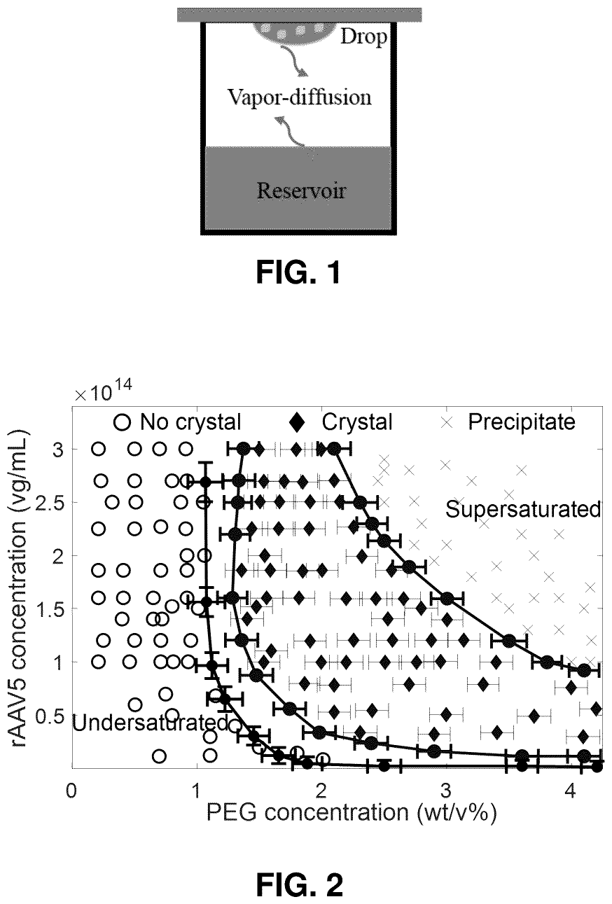

Crystallization conditions for rAAVs were screened in a hanging-drop vapor-diffusion experiment (FIG. 1) using VDX 24-well crystallization plates with glass cover slips at the top (Hampton Research, California, USA). Each well contained two liquid solutions: one being a droplet of small volume of 2 μL suspended from a glass cover slip at the top of the well, and the other being a reservoir of 1 mL at the bottom of the well. Each drop and reservoir contained polyethylene glycol (PEG) 8000 and sodium chloride (NaCl) as precipitants dissolved in a phosphate-buffered saline (PBS) solution. The reservoir was to ensure that the liquid conditions in each droplet would reach quasi-steady conditions sometime after closing the system (FIG. 1).

Each droplet was a mixture of 1 μL of AAV sample and 1 μL of buffer solution. Initial screenings were performed by varying the concentration of PEG8000 (from 0.1 to 7 w/v %) with a constant NaCl concentration. Subsequent screenings were conducted by varying the NaCl concentration to fine-tune the ionic strength of the crystallization medium. As time progresses, water evaporates from the droplet. Sometime after the supersaturation is reached, the nucleation of capsid crystals begins and eventually a vapor-liquid equilibrium is achieved at which point no further reduction in the volume of the droplet takes place.

Each droplet was monitored at regular intervals for a period of 1 to 2 weeks via an optical microscope (Imaging Source DMK42BUC03) to track the evolution of crystals. The particles were observed in real time using a microscope with an in-built CCD camera (Leica Z16 APO), using both normal and polarized light. Nucleation was found to reach completion within the first three days, which was followed by growth of the crystals for up to 5 to 6 days; at which point no further change in the crystals was observed. Finally, the remaining volume of the droplet was collected by a micropipette and then crystals and mother liquor were separated in a microcentrifuge operating at 8000 g for 120 s. Thereafter, the supernatant was collected by micropipette, diluted, and taken for Ultraviolet-Visible (UV-Vis) analysis in a nanodrop one C (Thermo Scientific) to measure the absorbance ratio A260/A280. Correction in the absorbance recorded in UV-Vis was made for absorbance due to Rayleigh scattering by subtracting the absorbance due to Rayleigh scattering from the total absorbance. The Rayleigh scattering coefficient was calculated by fitting the measured absorbance above 320 nm, where capsid protein and DNA do not show any molar absorbance. The path length for light recorded by the detector was 1 mm. Since rAAVs are stable (low degradation) in the pH range between 5.5 and 8.5 (Gruntman, A. M. et al., Hum Gene Ther Methods 26, 71-76 (2015); Lins-Austin, B. et al., Viruses 12, 1-18 (2020)), this experimental study was limited within that range. All experiments were performed at room temperature, 23±2° C.

### Construction of Phase Diagram

To find crystallization conditions (i.e., the concentration of precipitants PEG and NaCl at a specific pH), where only either full or empty capsids are crystallized, >500 conditions are screened for crystallization of both “full” and “empty” capsids samples. At each tested condition, the AAV either remain in solution, form an amorphous precipitate, or form crystals. A phase diagram is constructed by plotting this data as a function of PEG and NaCl concentrations. Separate phase diagrams are determined for “full” and “empty” capsids at different pH values. The whole parameter space is discretized into different regions where each region represents an experimental condition. This approach allows exploring one parameter at a time keeping other parameters constant. Based on the outcome of an experiment, the next experiment is performed at higher concentration (higher spacing) or lower concentration (lower spacing).

### Crystallinity Confirmation

X-ray diffraction (XRD) is the most commonly used method to confirm the crystallinity of a material.^{17,19,20,32} Because XRD requires a relatively large sample mass (˜0.2 g), crystallinity is determined by three different alternative methods: (i) light polarization, (ii) selected area electron diffraction (SAED) analysis using transmission electron microscopy (TEM)³²⁻³⁴²⁹⁻³¹ and (iii) scanning electron microscopy (SEM) imaging of crystals in some crystallization conditions.

### Polarization Study

Due to the large number of screened experimental conditions and length of experiments, it is not possible to screen crystals in each droplet using either XRD or SEM imaging. Therefore, the birefringence property of anisotropic crystals under cross-polarized light was used to screen the large number of crystallization conditions. Under cross-polarized light, isotropic crystals do not show any birefringence and the crystals appear dark (extinct). Similarly, materials that are not crystals (e.g., precipitate) do not show any birefringence. Screening of crystallization conditions based on birefringence is much simpler and faster than destructive methods such as SEM and TEM, making the approach suitable for repetitive screening of a large number of conditions. A 24-well plate was positioned on a Leica Z16 APO microscope base and each droplet was observed. Micrometer-sized crystals can readily be viewed under this microscope. Then plane-polarized light was passed through the crystals and the analyzer, which is also a polarizer, was rotated 360° while keeping the crystal's position fixed. On rotation of analyzer, anisotropic crystals will assume a spectrum of interference colour except at each every 90° position of the analyzer, where the interference is extinct and the crystals appear dark. This birefringence confirmed the crystallinity of particles.

### Cryo-TEM of AAV Crystals to Obtain Electron Diffraction Pattern (SAED)

For TEM imaging, each droplet containing well-developed crystals with sharp edges was selected. A 1:1 mixture (10 μL) of reservoir solution and 1× PBS buffer (pH 7.2) was added into the droplet. Then these crystals were observed under microscope, agitated to detach from glass surface and intentionally broken into smaller pieces using a micropipette tip with continuous microscopy monitoring. These protein crystals are not as durable as salt crystals and can be broken into small pieces on application of slight pressure using the micropipette tip. Then broken crystals were pre-fixed by adding 2.5% aqueous glutaraldehyde solution to inactivate the capsids' biological activity. In sample preparation for cryo-electron microscopy, 3 μL pre-fixed solution of broken crystals was dropped on a lacey copper grid coated with a continuous carbon film and blotted to remove excess sample without damaging the carbon layer by Gatan Cryo Plunge III. The grid is mounted on a Gatan 626 single tilt cryo-holder equipped in the TEM column. The specimen and holder tip were cooled down by liquid nitrogen, and cryogenically maintained during transfer into the microscope and subsequent imaging. Imaging on a JEOL 2100 FEG microscope is done using a minimum dose method that was essential to avoid sample damage under the electron beam. The microscope was operated at 200 kV and with magnification in the ranges of 10,000 to 60,000 for assessing sample size and distribution. Both images and diffraction patterns were recorded on a Gatan Orius SC200D (833).

### SEM Imaging of AAV Crystals

For SEM analysis of AAV crystals, conditions that produce well-developed crystals with sharp edges were considered. The droplet was diluted with equal volumes (5 μL each) of reservoir solution and 1× PBS buffer solution (pH 7.2) to prevent the dissolution of crystals and facilitate manipulations. The solution was agitated carefully to detach the crystals from glass surface without breaking the crystal. Then the crystals and solution were aspirated and dispensed on a centrifugal filter of 0.1 μm pore size (Millipore Sigma) and centrifuged once at 2000 g for 1 min to remove the free capsids and solution. Then the membrane filter paper was cut out of the tube frame and attached on a carbon tape on a brass stub. Then a 10-nm thick gold coating (EMS Quorum, EMS 150T ES) was applied on the crystal sample to make the surface conducting and then taken for SEM analysis. SEM analysis was performed in a high-resolution electron microscope (ZEISS merlin).

### EDX Analysis

EDAX analysis of crystals was performed (EDAX, AMETEK materials analysis division) using an Octane Elect Super Detector attached with SEM microscope (ZEISS Merlin). Sample preparation remained the same as for SEM analysis except there was no gold coating on crystals for EDAX analysis. For EDAX analysis, a crystal was first selected and then an elemental mapping was done on the area, which includes both crystal and the underlying membrane surface. Elemental mapping was performed on the membrane surface as a control. Finally, line and point EDAX is performed on different locations on a crystal. For all the analysis, more than 100 scans were used unless mentioned. Crystals analysed in EDAX are more than 10 μm thick and a beam of 15 kV was used for this analysis.

Room Temperature TEM Analysis of AAV Particles after Crystallization

To visualize and measure the percentage of full and empty capsids in the crystals as well as to understand whether the virus particle's morphology remains the same, room temperature TEM imaging was performed. Crystals were collected following the procedure in the “Cryo-TEM of AAV crystals to obtain electron diffraction pattern (SAED)”, section and dispensed into a centrifugal filter paper of 0.1 μm pore size and centrifuged at 2000 g for 1 min at ambient temperature. The crystals remained attached to the filter paper, effectively removing free AAV particles, PEG, and NaCl that remained in the supernatant. The crystals were washed twice (100 μL of 1:1 mixture of reservoir solution and 1× PBS) to ensure the complete removal of residual virus capsids and diluted the PEG and NaCl. The number of washing steps may vary based on the type of crystals in the droplet as AAV crystals are very soft and prone to breakage even with low shear. For small crystals, more than one/two washing steps cause breakage and eventual passage of crystals through the filter membrane pores. Then the crystals were washed once with 100 μL of diH₂O (≥18 mW, MilliQ) and centrifuged at 2000 g for 1 min to remove the adsorbed PEG and NaCl from the filter. The centrifugation was performed immediately after addition of diH₂O as crystals are highly soluble in water. The AAV crystals were dissolved by incubating in 100 μL of diH₂O that was added into the filter (for 30 min at room temperature). The solubilized capsids are fixed by adding 2.5% aqueous glutaraldehyde solution to inactivate the capsids.

Virus particles were imaged by TEM (JEOL model) at room temperature at the MIT TEM facility (Koch Institute, MIT). For sample preparation for TEM imaging, 10 μL of dissolved crystal solution was aspirated and dropped on a 200-mesh copper grid coated with continuous carbon film and left at room temperature for 10 minutes. After 10 minutes, any remaining water on the grid was absorbed by a tissue paper (Kimwipes) and 10 μL of 1% aqueous phosphotungstic acid solution is immediately dropped onto the moist TEM grid. After 30 s, the excess liquid was absorbed by a tissue paper. The grid was left at room temperature for 45 minutes to allow the remaining liquid to dry before inserting the grid into the TEM chamber. The image collection procedure was the same as for cryo-TEM imaging (Section 2.3.2.2). All images were recorded on a Gatan 2k×2k UltraScan CCD camera. Empty capsids show the contrast difference as shown in the results and discussion section.

Crystal Collection, Dissolution and Stock Solution Preparation for Biological Analysis (ELISA, ddPCR, Cell Infection, Flow Cytometry, SDS PAGE, Mass Photometry)

Stock solution of rAAV is prepared by dissolving crystals for biological assays. To prepare AAV stock solution, crystals were collected from the droplet following the same procedure in Section “Cryo-TEM of AAV crystals to obtain electron diffraction pattern (SAED)”. The collected crystals were collected and washed according to the procedure described in Section “Room temperature TEM analysis of AAV particles after crystallization”. The AAV crystals were dissolved by incubating in 300 μL of nuclease-free water at 4° C. refrigerator for 30 min. The dissolved crystal solution was transferred into an Amicon centrifugal filter (10 kDa NMWCO) and centrifuged at 10000 g for 5 min. Capsids were then washed 3 times using 200 μL of nuclease-free water at 10000 g for 5 min each time to ensure complete removal of remaining salt and precipitant. Harvested capsid particles were then resuspended into 200 μL of Dulbecco's 1× PBS (DPBS without magnesium and calcium) and collected into a vial and stored at 4° C. for short-term use. For long-term storage, 5% Sorbitol solution is added into the capsids suspension and stored in a cryovial at −80° C.³⁵

### ELISA Experiment

ELISA assay protocols followed the manufacturer's instructions (Progen) for rAAV5 (AAV5 Xpress ELISA kit) and AAV9 (AAV9 Xpress ELISA kit). Briefly, to determine a workable capsid concentration for ELISA assays, a range of concentrations was produced by serial dilution of the AAV stock solution (see Section on stock solution preparation): (1×, 2×, 4×, 8×, 16×, 32×, 64×, 128×, 256×, 512×, 1024×). Capsid concentrations outside the workable range, will either lead to signal saturation or low signal when the concentration is too high or too low, respectively. Assay buffer (ASSB 20×, Progen) was diluted to ASSB 1× using diH₂O (milli-Q) Similarly, (lyophilized) capsid standards, known as ‘kit control’ (KC), are included in the ELISA kits. The KC was reconstituted using ASSB 1× (500 μL) and a serial dilution (1×, 2×, 4×, 8×, 16×, 32×, 64×) was prepared using ASSB 1×. Anti-AAV5 mAb-biotin conjugate is dissolved in ASSB 1× (750 μL) to produce 20× Biotin solution. For both KC dilution and sample dilution, duplicates were prepared. KC dilution (100 μL) and AAV stock dilution (100 μL) were loaded on ELISA plate and the assay was performed following the procedure as recommended by the ELISA kit manufacturer. At the end of the assay, absorbance of the solution in each well is read immediately in a microplate reader (Synergy H1; BioTek Instruments, Winooski, VT, USA) at 450 nm wavelength. Absorbance readings for the sample dilution as well as KC dilution were corrected by subtracting the absorbance of the control. The averaged absorbance values, from duplicates, for the KC dilution (along the y-axis) were plotted against the corresponding concentration along the x-axis and a 4-parameter logistic (4PL) fit is performed in Matlab to calculate the concentration of the capsids in the sample.

ddPCR (Droplet Digital PCR) Assay

The quantity or titer of genomes was determined using ddPCR assay. Like ELISA, a serial dilution (1×, 2×, 4×, 8×, 16×, 32×, 64×, 128×, 256×, 512×, 1024×) of AAV stock solution (see section “Crystal collection, dissolution and stock solution preparation for biological analysis (ELISA, ddPCR, cell infection, flow cytometry, SDS PAGE, mass photometry”) was performed using DNase-free water. A master mix solution (18.2 μL for each well) was prepared by mixing 7.79 μL of 2.62% DMSO, 0.41 μL of primer mix (forward and reverse), and 10 μL of EvaGreen Supermix (QX200 ddPCR EvaGreen Supermix; BIO-RAD). In each well of the 96-well PCR plate, diluted AAV stock solution (2.2 μL) and the master mix (18.2 μL) were loaded to make a total volume of 20.4 μL. Thermal cycles used for the PCR reaction were standard cycles used for EvaGreen Supermix as set by BIO-RAD. The enzyme activation step was performed at 95° C. for 5 min and 1 cycle. The next step, denaturation was performed at 95° C. for 30 s and 40 cycles. The third step, annealing/extension was performed at 60° C. for 1 min and 40 cycles. The fourth step, signal stabilization, was performed first at 4° C. for 5 min for 1 cycle and then at 90° C. for 5 min for 1 cycle. Finally, the sample was held at 4° C. before being removed from cycler.

Forward primer was 5′-GCAAAGACCCCAACGAGAAG-3′ (SEQ ID NO: 1) and

reverse primer was 5′-TCACGAACTCCAGCAGGACC-3′ (SEQ ID NO: 2). DNase free water was used as the non-template control and for each dilution, duplicate sample is prepared. The number of viral DNA copies in the stock solution and in the crystals are back calculated from the ddPCR data.

### Mass Photometry Experiment

Mass photometry measurements are performed on a SamuxMP instrument (Refeyn Ltd., MA, USA), which has a higher resolution tailored especially for AAV vector analysis. Prior to each measurement, a calibration is performed using “empty” AAV9 vector (3.74 MDa). The molar mass of the “empty” AAV9 is provided by Refeyn Ltd. (MA, USA). To generate the calibration curve, 10 μL of 1×PBS is pipetted into a well of silicon gasket, the focus is adjusted automatically, and then 10 μL of AAV9 calibrant is added into the loaded PBS. Before measurement, the loaded sample and PBS are mixed vigorously by aspirating and dispensing multiple times carefully to avoid formation of bubbles. The measurement time is set to 60 s, which captures the binding and unbinding events in the form of a movie. The measurements are recorded using AcquireMP 2.4.2 (Refeyn Ltd., MA, USA) and analyzed with DiscoverMP (v2023 R1.2) (Refeyn Ltd., MA, USA). For all the measurements, the binning width is set to 40, and the ratiometric contrast distribution is fitted by a Gaussian function to obtain the molecular weight of subpopulation. The F/E ratio (full capsid to empty capsid ratio) and linearity are visualized using MATLAB.

### SDS-PAGE Electrophoresis Analysis and Western Blot Analysis of Capsid Proteins

SDS-PAGE analysis is performed to find proteins present in the AAV sample and if the viral proteins remain preserved after crystallization. Viral proteins are denatured by incubating a mixture of AAV sample, 2-mercaptoethanol, and diH₂O (2:1:2 volume ratio) at 95° C. for 10 minutes. The denatured samples and the protein ladder (BenchMark™, Thermofisher) are resolved electrophoretically by through a 4-20% polyacrylamide gel (BIO RAD TGX gradient gel in a Mini-PROTEAN tetra electrophoresis cell/chamber) in 1× Tris/glycine/SDS buffer at 100 V for 100 minutes. For silver staining, fixative enhancer solution, development accelerator solution, silver staining solution (7:1:1:1 mixture of diH₂O, silver complex solution, reduction moderator solution, and image development solution), and stop solution (5% acetic acid solution) are prepared and gel is treated as per manufacturer's instruction (BIO RAD), except the final staining step, where gel is stained for 7-10 minutes only. The silver-stained gel is imaged in ChemiDoc imaging system (BIO RAD). Similarly, Western blot experiment was performed following the protocol as suggested by BIO RAD and image was collected in ChemiDoc system (BIO RAD). For western blot analysis, standard procedure was followed as suggested by BIO-RAD.

### Cell Biological Activity Measurement

### Preparation of Adherent Cell Culture

Adherent cell culture is prepared for cell transduction experiments to check whether biological activity of AAV remains preserved after crystallization. Refrigerated (−20° C.) fetal bovine serum (FBS) is thawed, heat inactivated (at 57° C. water bath for 30 minutes), equilibrated (at 37° C. water bath), and then filtered to remove suspended insoluble materials/aggregates). Cryopreserved HEK293T cells are thawed (2 min at 37° C. water bath) and transferred into a centrifuge tube that contains 9 mL of complete growth medium (10 v/v % FBS in DMEM). The cells are then pelleted (centrifuged at 1000 g for 5 minutes) and resuspended in complete growth medium three times to remove the DMSO used in cryopreservation. The cell pellet is then resuspended in the preconditioned complete growth medium (9 mL at 37° C. in CO₂ for 15 min), dispensed in a 10-cm cell culture dish (Corning tissue culture-treated culture dishes), and then incubated at 37° C. in 5% CO₂ atmosphere (passage 1). The culture is observed under microscope twice daily.

Adherent cells, 80% confluent, are detached from the cell culture dish using trypsin solution (3 mL of 0.25% Trypsin-0.53 mM EDTA (1×) for 10 min) and transferred into centrifuged tube after addition of complete growth medium (10 mL). Cells are then pelleted (centrifuged at 1000 g for 5 min) and resuspended in complete growth medium (3 mL) thrice to remove trypsin. Finally, cell pellet is resuspended into 1 mL of complete growth medium and cell density is measured in a Countess II cell counter (Invitrogen, MA, USA) with trypan blue (0.4% stock) as a dye in 1:1 ratio.

A subculture was prepared following the same procedure as described for passage 1 and the remaining cell suspension is cryopreserved in 10% DMSO solution for future use.Study of HEK293T cell transduction by rAAV present in crystals.

For study of cell transduction, cells from passage 1 (2 mL in each well of a Corning Costar TC-treated 6-well plate) are transduced with AAV stock solution (prepared as discussed above) for multiplicity of infection (MOI, number of viral genome-containing particles per cell) of 10⁴, 10⁵, and 10⁶. Cells are then cultured in an incubator at 37° C. in 5% CO₂ atmosphere and observed in a fluorescence microscope once daily for 5 days. On day 5, viable cell density and percentage of GFP-positive cells are measured in a Countess II Cell Counter with trypan blue (0.4% stock) as a dye in 1:1 ratio to assess cell viability. Transduction efficiency, as a percentage, is calculated by dividing GFP-positive cells by the total cell number×100%.

### TCID50 Experiment

TCID50 experiments were performed to obtain a more precise value for the infectious viral titre. In this experiment, ˜5000 cells (HEK293T cells from passage 1, Section “Seeding Experiments”) were seeded in each well of a 96-well plate (Corning Costar TC treated) and cultured in complete growth medium (100 μL 10% FBS in DMEM) for 2 days at 37° C. and 5% CO₂. A dilution series of AAV stock solution (Section “Crystal collection, dissolution and stock solution preparation for biological analysis (ELISA, ddPCR, cell infection, flow cytometry, SDS PAGE, mass photometry”) was prepared using complete growth medium (10% FBS in DMEM) in a 96-well plate (Corning Costar V shaped) with dilutions of 10⁻³, 10⁻⁴, 10⁻⁵, 10⁻⁶, 10⁻⁷, 10⁻⁸, 10⁻⁹, and 10⁻¹⁰. On day 2, 100 μl of each dilution is added to each well in a column except for the first two columns, which are used as a negative control. The cells are then cultured (at 37° C. and 5% CO₂) and cell growth and GFP fluorescence are examined under a microscope daily for 5 days. On day 5, for each dilution, % of GFP-positive wells are measured. The TCID50 is calculated based on Reed-Muench method.³⁶

### Flow Cytometry Experiment

### Suspension Cell Culture Preparation

Flow cytometry was performed to obtain a more accurate measurement of cell viability and percentage of GFP-positive cells than that in previous transduction experiment. Cryopreserved HEK293 cells were thawed at room temperature and dispensed into 500 μL pre-warmed (at 37° C. and 5% CO₂) FreeStyle 293 expression medium and cultured in a Corning Erlenmeyer cell culture flask in 20-mL FreeStyle 293 expression medium. Cell viability and viable cell density are measured once daily in a Countess II Automated Cell Counter using trypan blue (0.4% stock) as a dye in a 1:1 ratio to maintain a cell viability above 90%. Cells are passaged when cell density reaches 2-3 million/mL.

### Flow Cytometry Experiment

For flow cytometry experiments, a total of nine subcultures were prepared by adding 1.1 mL HEK293 cells (from passage 21 with 99% viability) in 50 mL FreeStyle 293 medium (at 37° C. and 5% CO₂) in each Erlenmeyer shake flask (Sigma Aldrich). Out of the nine subcultures, one shake flask serves as a negative control and four shake flasks serves as a positive control, where cells were transduced with standard AAV sample (Virovek) for MOIs (concentration of virus to concentration of living cells)) of 10⁶, 5×10⁵, 10⁵, and 10⁴. In the remaining four shake flasks, cells are transduced with the AAV stock solution (Section “Crystal collection, dissolution and stock solution preparation for biological analysis (ELISA, ddPCR, cell infection, flow cytometry, SDS PAGE, mass photometry”) in the same MOIs as in the positive control. Cells were cultured (at 37° C., 5% CO₂, and 135 rpm) and collected every 24 h for flow cytometry experiments. Cell suspension (0.5 mL for each experiment) from each flask is first made free from aggregates and debris by pushing through nylon mesh in polystyrene tube, and then viability and % GFP-positive cells were measured in Aria 4 (Flow core, Koch Institute, MIT) using PI (propidium iodide) as a viability dye.

### Seeding Experiments

Nucleation (primary nucleation) followed by growth of crystals in hanging drop vapor diffusion experiments (in a 24-well plate) takes about 2 weeks to complete. For the crystallization method to be useful as an alternative purification method, the process needs to be completed within a time comparable to that of existing technologies. To evaluate the feasibility of crystallization for AAV purification, a crystal seed growth experiment was performed. Seeding was used to reduce the overall time scale by not needing any time for nucleation. In a seeding experiment, purified/precleaned crystal seeds were added into the crystallization medium at a certain time. To mimic the seeding experiment, a droplet containing well-grown crystals was identified, and growth solution (2 μL of 1:1 ratio of reservoir solution and AAV Virovek sample) was added. The variation in size of crystals was measured at 5 to 30 minutes intervals in a microscope (Leica Z16 APO), and the time required for the crystals to grow to a size (minimum 10-100 μm for it to be separable in industrial filters) was measured.

### Scale-Up Experiment

To understand whether the preferential crystallization is relatively scalable, a microliter droplet-based system was scaled up to a milliliter-volume crystallizer, popularly known as the Crystalline, which is widely used for small-molecule pharmaceuticals. Like the hanging drop system, the scale-up crystallizer also has an evaporation capability but, due to the flow of inert gas through the crystallization chamber, evaporation is faster in the latter. Unlike the hanging droplet system, the scaled-up reactor has a cooling jacket and an in-built stirrer. For this experiment, crystallization solution (5 mL 1:1 mixture of the rAAV sample and precipitant solution) was incubated at 30° C. for 3 hr followed by cooling at 10° C. and holding there for 24 hr. Solution was simultaneously evaporated by passing N₂ gas throughout the chamber. A small volume (100 μL) of the solution was observed under microscope and ddPCR and ELISA experiments are performed.

### Yield Calculation

For the calculation of yield, droplets containing only a few distinct large crystals with no precipitate were selected and a small volume (5 μL) of reservoir solution was mixed thoroughly with the original droplet solution. Then 1 μL of the solution was collected, diluted 1000× by dispensing into PBS buffer, and analyzed to determine the quantities of total capsids (ELISA) and full capsids (ddPCR). Subtracting full capsids from total capsids determines the empty capsid titre. The yield is the difference between the final and initial quantity of capsids divided by the initial quantity of capsids.

### Results and Discussion

PEG is one of the most widely used precipitants in the crystallization of proteins because of its well-known local solubility reduction by the so-called volume exclusion effect.³⁷⁻³⁹ Both PEG6000 and PEG8000 have been used in rAAV capsid crystallization. It has been observed that, the higher the molecular weight of the PEG, the lower quantity needed to reach a local supersaturation.¹⁷⁻²⁶ PEG8000 was used, as in most past studies, with an initial concentration of about 2 to 7%, wt: vol, to nucleate rAAV capsid crystals in a variety of buffer solutions. A solubility analysis shows that the visible particles formed in this study are composed of protein (capsids) rather than NaCl or PEG. SEM imaging/polarization/SAED analysis shows that the particles that form are crystalline rather than amorphous.

An analysis was conducted to rule out the possibility that any of the particles formed in this study consist of NaCl or PEG. A range of NaCl concentrations (0.01 to 2.5 M) and PEG concentrations (0.5 to 8% wt: vol) were evaluated. To determine whether the coformulation of NaCl and PEG affects the solubility of either component,⁴⁰⁻⁴³ a set of control experiments were carried out for the combinations across the full ranges of PEG concentration (0.5 to 8 wt/v %) and NaCl concentration (0.005 to 2.5 M) in the absence of rAAV capsids. No crystals were observed under these conditions, confirming that rAAV capsids were essential for crystallization.

A wide range (0.5 to 8 wt/v %) of PEG8000 concentrations was explored to search for conditions to preferentially nucleate crystals of full capsids (FIG. 2). rAAV5 crystals nucleated at lower initial PEG8000 concentrations than in previous reports,¹⁷⁻²⁶ which may be attributed to the use of a different buffer in the present studies.

The difference in solubility curves for full and empty capsids are within the error bars. The full capsids have a higher molecular weight, which would tend to reduce solubility, but have a higher charge, which would tend to increase solubility. FIG. 2. The location of the DNA, which contributes both mass and negative charge, within the capsid may limit its influence on solubility.

The range of PEG8000 concentration over which crystallization occurs is somewhat wider than that reported for rAAV2¹⁷⁻²⁵ (FIG. 2). Similar to rAAV2¹⁹, the width of the metastable zone narrows as the concentration of rAAV5 and rAAV8 increases. rAAV5 is found to crystallize over a broader range of PEG8000 concentration than that of rAAV8.

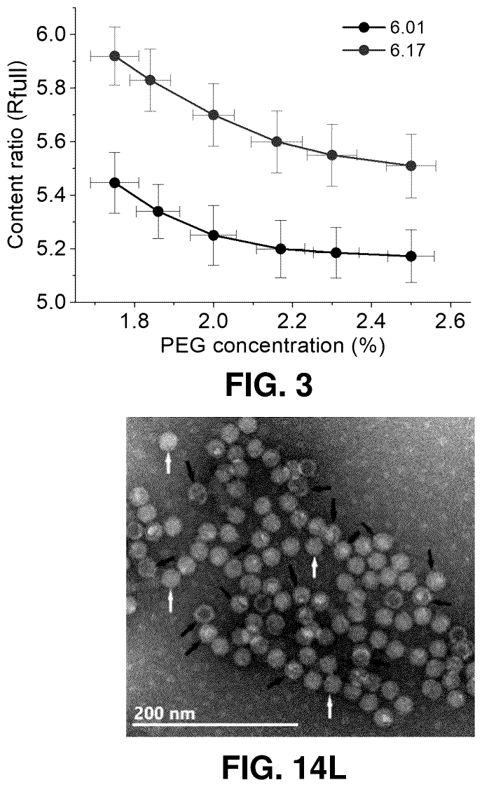

A sample of “empty” rAAV5 (92% empty, 8% full) was crystallized and the quantity of full rAAV crystallized was determined using UV-Vis spectroscopy of the droplet solution supernatant (FIG. 3) as a function of PEG concentration and pH at a specified NaCl concentration. This “empty” rAAV5 sample was crystallized and UV/vis spectroscopy (FIG. 3) was performed to measure the percentage of full rAAV in the supernatant relative to the total (combined full and empty) rAAV capsids, known as content ratio, which was computed from⁴⁴

$${R}_{full} = \frac{{ϵ}_{260}^{capsid} - \frac{{A}_{260}}{{A}_{280}} {ϵ}_{280}^{capsid}}{\frac{{A}_{260}}{{A}_{280}} {ϵ}_{280}^{DNA} - {ϵ}_{260}^{DNA}} ( 100 \% ) , \qquad ( 1 )$$

with extinction coefficient ratio of 0.5 and 2 for capsid and virion DNA, respectively, as reported in the literature.⁴⁵⁻⁴⁷ This expression corrects for Rayleigh scattering.^{44,45-47}The content ratio was about 5% to 6% over a wide range of PEG concentration and at two pH values (FIG. 3), indicating a higher depletion of full capsids than empty capsids from the supernatant. In other words, the full capsids were preferentially crystallized, removing full capsids from the solution. As the PEG8000 concentration increases, the content ratio decreases, i.e., more full capsids are crystallized from the supernatant. Similarly, lower pH favors the crystallization of full rAAV. This variation in content ratio with pH and PEG concentration implies that the crystallization rate of full capsids is much higher than empty capsids at that specified experimental condition. At each of the higher PEG concentrations evaluated, the content ratio decreases at a slower rate compared to lower PEG concentration regimes, indicating a limiting PEG concentration beyond which no further enhancement of full rAAV crystallization occurs. The reduction in content ratio is similar for pH of 6.01 and 6.17.

While the solubilities of full and empty capsids are nearly the same (FIG. 2) and both crystallize over a bounded region of PEG concentration (FIG. 2), full capsids crystallize at faster rates than empty capsids over a certain range of PEG concentration. The differential rates of crystallization were further investigated. To establish a higher resolution “picture” of crystallization conditions, a wide range of precipitant, pH, and salt concentrations were screened (FIG. 4). The inorganic precipitant, NaCl, influences the crystallization process by shifting the protein solubility whereas the organic precipitant, PEG, functions as an aquacide effectively concentrating the proteins. Sodium chloride is widely used as a precipitant in the crystallization of proteins^{37-39,48,49} to shift the protein solubility by changing the number of water molecules available for interaction with the charged part of a protein's surface (A reduction in protein solubility (aka salting out) is associated with a decrease in the number of water molecules, whereas an increase in protein solubility (aka salting in) is associated with an increase in the number of water molecules.^{65,67,102-104}). While NaCl was added in previous AAV capsid crystallization studies,¹⁷⁻²⁴ this study is the first to quantify the effect of the variation in NaCl concentration on the crystallization of AAVs. As such, this study is also the first quantification of the effect of variation in ionic strength.

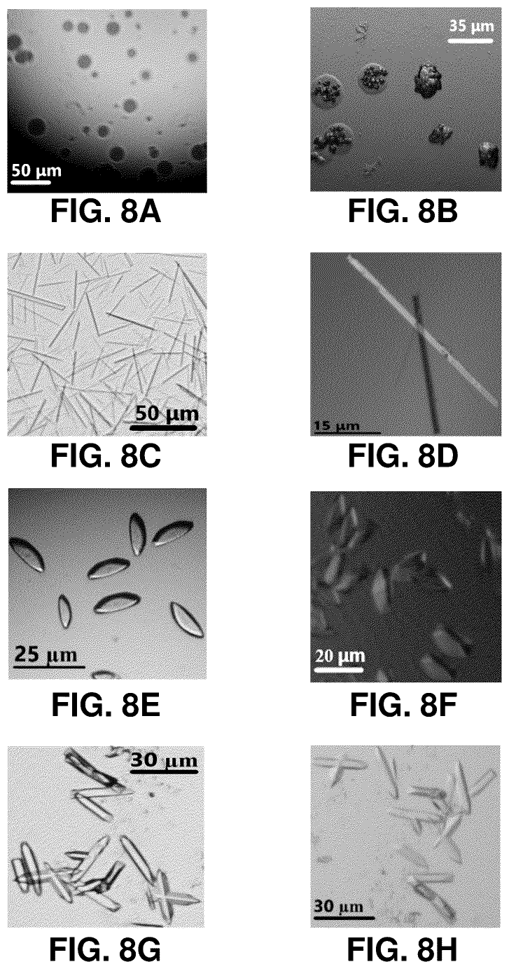

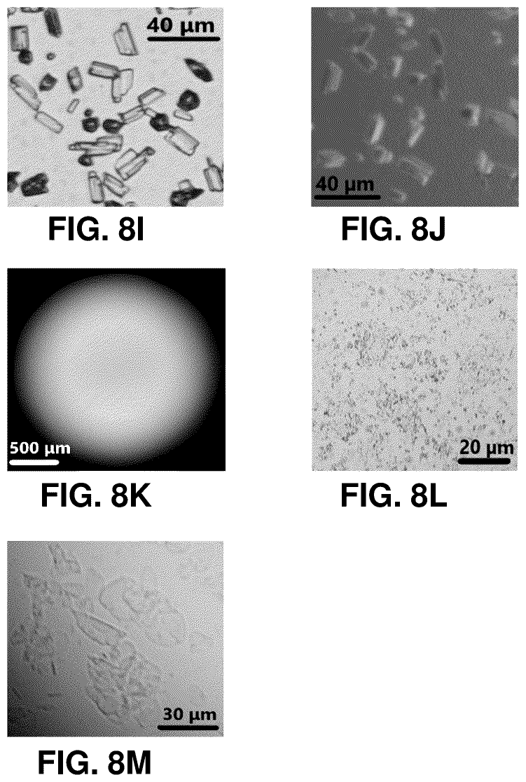

Crystallization conditions were explored for two serotypes: rAAV5 and rAAV9. FIGS. 4A-7A are crystallization phase diagrams for “full” and “empty” capsids of rAAV5 and rAAV9 as a function of pH and PEG and NaCl concentrations. The graphical representation reveals that each region in which crystallization occurs is closed and bounded. Crystallization can occur over a large range of PEG concentration; for each value of PEG concentration within this range, there is an upper and a lower limit of NaCl concentration for which crystallization occurs. Empty and full capsids of different serotypes show appreciably different solution behaviour at different NaCl concentrations, with the behaviour being a strong function of the PEG concentration, which is discussed later in this section. While AAV capsids have been crystallized previously,¹⁷⁻²⁵ past efforts have not included the analysis of phase behaviour. At NaCl concentrations below a certain threshold, a variety of situations of non-crystallization outcomes can occur including forming a dispersed phase (gel spots as in FIG. 8A), undefined cluster-like spots (as in FIG. 8M), precipitates (FIG. 8L), and clear solution, which are discussed later in this section. The solution remains clear at high NaCl concentration (FIG. 8K).

The optical properties of the crystals were assessed using normal and cross-polarized light microscopy. Crystals appear as colorless or grey in a normal light microscopy images (FIGS. 8C, 8E, 8G, 8I). When crystals are observed under cross-polarized light, crystals remain dark/extinct (data not shown) at every 90° position (where two polarizers' vibration directions are perpendicular to each other) and show interference coloration (FIGS. 8D, 8E, 8H, 8I) on rotation due to the splitting of the transmitted light into two rays: slow (e) and fast moving (o) rays.50-52 These photos indicate that the crystals are optically anisotropic (i.e., the speed of light varies with direction) and birefringent (i.e., depends on the polarization and propagation direction of light). While cross-polarized light is used in optical minerology and crystallography to identify crystals, this study appears to be the first to study rAAV crystals under polarized light. The value of the birefringence—the maximum difference in refractive index of the material (|nₑ−nₒ| for the crystals from the Michael Levy chart) was calculated.⁵⁰ This calculation gives birefringence values, Δn, of ˜0.36 (FIG. 8D), ˜0.14 (FIG. 8F), ˜0.16 (FIG. 8H), and ˜0.18 (FIG. 8J), indicating higher order birefringence. The retardation (i.e., the optical path difference, Γ=d|nₑ−nₒ|) of the slowest ray with respect to the fastest ray^{50,52}) of the slowest ray with respect to the fastest ray is found to be ˜630 nm (FIG. 8C), ˜1120 nm (FIG. 8F), ˜1280 nm (FIG. 8H), and ˜2260 nm (FIG. 8F). This range of medium to higher order of retardation suggests that the difference in refractive index, and hence the difference in structural and electrical environment in different directions, is significant. The presence of a unique angle of extinction and birefringence in the crystals in this analysis suggests that the particles are single crystals, in contrast to polycrystalline materials that behave anisotropically.^{50,53} The literature suggests that NaCl generally forms cubic crystals and does not show any interference colours under cross-polarized light as NaCl crystals are optically isotropic.^{50,52} All AAV capsid crystal particles generated in this study are optically anisotropic, and are not cubic in shape, which indicate that the particles are not NaCl crystals.

The width of the crystallization zone varies with both pH and PEG concentration (FIGS. 4B-7B). For “full” AAV5, the crystallization zone is narrowest at low PEG concentration and is widest for intermediate PEG concentration. This variation in the width of the crystallization zone is largest at low pH (5.7). At PEG concentration less than ˜1.8 wt/v % for any NaCl concentration, the solution remains clear (FIG. 4A). At low NaCl concentration (e.g., ˜0.3 M), as PEG concentration increases (e.g., to ˜2 wt/v %), circular spots form (indicating a phase separation). On further increase in PEG concentration (e.g., to ˜3 wt/v %), the solution remains clear, and at even higher PEG concentration, precipitation is observed (FIG. 4A). The formation of spots and clear solution are explained below. A variety of crystal morphologies are observed for “full” rAAV5 capsids. Intermediate PEG and NaCl concentrations (e.g., 3 wt/v % PEG and 1 M NaCl) favor the formation of needle-shaped crystals, whereas higher PEG concentration (e.g., 3.7 wt/v % PEG) favors the formation of rod-like and cylindrical crystals (FIG. 4A).

For “empty” rAAV5 capsids, the range of NaCl concentration over which crystallization occurs is shifted to lower values (FIG. 5A). As for “full” rAAV5, the crystallization zone moves to the higher NaCl concentration as the PEG concentration increases. The overall area (in the NaCl-PEG plane) over which crystallization occurs increases with increase in pH (FIG. 5B), which is not significant for “full” rAAV5 (FIG. 4B). AAV5 “empty” crystallize over a narrow range of PEG concentration, at about 1.2 wt/v %, without the addition of slat, whereas “full” rAAV5 fails to form crystals in the absence of NaCl. As for “full” rAAV5, the solution remains clear at low PEG concentration, contains spots at intermediate PEG and low NaCl concentration, and contains precipitates at high PEG concentration (FIG. 5A). At high PEG concentration, AAV5 precipitates form. “Empty” rAAV5 mostly forms needle-shaped crystals at low NaCl concentration and, as the NaCl concentration increases, transitions to rod-like and then to cylindrical crystals.

The crystallization zone for “full” rAAV9 is narrow at low PEG concentration and the widest at high PEG concentration (FIGS. 6A-6B). This variation in the width of crystallization zone is largest at pH 5.9. At PEG concentration less than ˜2.7.5 wt/v % and for NaCl concentration less than ˜1.8M, star- and leaf-shaped particles are formed; precipitation occurs at PEG concentration higher than ˜4.75 wt/v % and for any NaCl concentrations; the solution remains clear at PEG concentrations below ˜1.5 wt/v % for any NaCl concentration and at NaCl concentrations greater than ˜2M for any PEG concentration. As for rAAV5, the “full” rAAV9 crystallization zone moves to the higher NaCl concentration as the PEG concentration increases. In contrast to rAAV5, “full” rAAV9 mostly forms complex crystal morphologies such as rod, cylinder, needle, orthorhombic, prism, and bladed (FIG. 6A), and the full range of morphologies occur throughout the crystallization range for “full” rAAV9.

For “empty” rAAV9, the crystallization zone is the widest for intermediate PEG concentration and narrowest for high PEG concentration (FIG. 7A). As with “empty” rAAV5, crystallization can occur at higher NaCl and PEG concentrations for some values of pH. The area of the region where crystallization occurs is smallest at low pH (5.7) and largest at pH 7.2 (FIG. 7B). With the “full” rAAV9, the solution remains clear at higher NaCl concentration and at lower PEG concentration, precipitate forms at high PEG concentration, and star and leaf-shaped particles are formed at low NaCl and intermediate PEG concentrations (FIG. 7A). Like “full” rAAV9, “Empty” rAAV9 mostly forms complex crystal morphologies such as bladed, prism, and pyramid, orthorhombic and these crystal morphologies can occur anywhere within the crystallization region, which are not observed in either “full” or “empty” rAAV5. Much higher PEG concentration is required to cause the crystallization of “empty” rAAV9 than for “empty” rAAV5 (FIGS. 7B and 5B).

Both “full” and “empty” rAAV9 do not form gel spots (FIGS. 6A and 7A), which are observed in both “full” and “empty” rAAV5 (FIGS. 4A and 5A). Unlike with “full” rAAV5, crystallization of both “full” and “empty” rAAV9 is impossible without the addition of salt (FIG. 7B). Star- and leaf-shaped particles formed in both “full” and “empty” rAAV9 are most likely opaque crystals, which do not show any birefringence under cross-polarized light and remains dark. Absence of a proper characterization technique and difficulty in separation made it impossible to characterize them. Thus, experimental conditions favouring the formation of star- and leaf-shaped particles are excluded from analysis.

The crystallinity of the particles formed in the vapor diffusion experiments was verified by SAED (data not shown) and SEM (scanning electron microscope) imaging (data not shown). The presence of the spot diffraction patterns in the reciprocal lattice is strongly supportive of rAAV capsid crystals formation. The absence of a ring pattern indicates that the particles are single crystals. For “full” rAAV5, the diffraction pattern indicates the presence of distinct crystal planes, whereas the electron diffraction pattern of “empty” rAAV5 indicates the presence of many other spots apart from distinct crystal planes. Such spots are likely due to impurities or crystal defects^{32,55,56}. Similar spots are observed for “full” rAAV9. For “empty” rAAV9, there is a distinct spot diffraction pattern, which is suggestive of the presence of some single. Indexing of each of these spots in the diffraction pattern requires the knowledge of unit lattice constants and angles, which are generally obtained from x-ray diffraction (XRD analysis)⁵⁴.

SEM image of particles at higher magnification were obtained (data not shown). As it suggests, for all serotypes, the particles are spherical balls. The size of these balls is same as that of capsids. Since there was no other spherical material of that size in the system except spherical capsid particles, these spherical balls are definitely capsids. These capsids were organized in a highly ordered manner, and this pattern of arrangement of capsids was repeated periodically over a long distance (long range order). Images also showed multiple layers of capsids representing each distinct plane. The presence of highly ordered capsid layers in the particle was a visual proof that these particles are definitely crystals of capsids, because amorphous solids are characterized by highly disordered structure.

At low PEG concentration, the clear solutions at all NaCl concentrations for full and empty capsids for both serotypes was due to the low volume exclusion effect of PEG and consequent undersaturation or low supersaturation inadequate to form crystals. At intermediate PEG and low salt concentrations for rAAV5, the formation of spots was observed (FIGS. 4A and 5A). The composition of the spots is attributed to “gel/oil” phase, which is a protein-rich phase (FIG. 8A). Crystals form in some of these spots (FIG. 8B) after a long period (e.g., 7-14 days) and other spots remain as they are. An increase in protein solubility at lower salt concentration agrees with calculations of the protein-protein potential of mean force in terms of second virial coefficients for different proteins with different salts, which associates the higher protein solubility with a decrease in attractive protein-protein interactions.⁶⁷⁻⁷¹ For the NaCl concentrations explored in this study, the ionic strength remains well below the maximum solubility limit found in the literature for NaCl^{65,66,72,73}. For NaCl concentration below this limit, the low solubility results in a high supersaturation, a very high driving force, and the formulation of a new protein-rich phase (dispersed phase) known as a “gel/oil.” The high rate does not provide enough time for the capsids to orient into a crystal lattice to form a crystal, hence a gel forms.

At high PEG concentration (e.g., 5 wt/v %), for both serotypes and all values of NaCl concentration, very high supersaturation associated with the very low solubility (FIG. 2) presumably leads to the formation of precipitate before the protein rich gel/oil phase can form. Precipitation occurred at high PEG concentration for “full” and “empty” capsids of rAAV5 and rAAV9. At high NaCl concentration at lower PEG concentration, the high capsid solubility is associated with undersaturation or very low supersaturation, so no new phase forms during the period of the experiment and the solution remains clear with no precipitate (FIGS. 4A-7A).

Within the crystallization region (FIGS. 4A-7A), the NaCl concentration is below its solubility in solution, and supersaturation favorable for crystallization is achieved. Moderate supersaturation is associated with a moderate driving force, which reduces the rate of formation of a new solid phase, giving the capsids sufficient time to orient/order so as to form crystals. These results are the first report for the crystallization of full rAAV5 and full AAV9. For both the serotypes, the range of PEG concentration in which crystallization occurs shifts to higher values with higher NaCl concentration for most values of pH (FIGS. 4B, 5B, 7B). This positive correlation between PEG and NaCl concentrations that induce crystal formation is likely associated with their opposite effects on the protein solubility over the experimental conditions in this study. As PEG concentration increases, the capsid solubility decreases (FIG. 2). On the other hand, as NaCl concentration increases, the capsid solubility increases under the experimental conditions. These competing effects would mostly cancel, so that the supersaturation remains at intermediate values conducive to crystallization^{19,59,77,78}.

Although the morphology of empty and full capsids appear equivalent, two physical property differences exploited in existing separation processes are the density (1.31 gm/cm³ for empty and 1.41 gm/cm³ for full capsid assuming a 4.7knt virion genome) and the isoelectric point (pI of 6.3 for empty and 5.9 for full) 10.11. However, separating empty from full capsids, especially at preparative scales, has been challenging due to these very small physicochemical differences. Ultracentrifugation of full and empty capsids exploits the difference in density, and ion exchange chromatography exploits the difference in charge density, between full and empty capsids. The difference in charge density forms the basis that crystallization could be potentially used for the selective purification of full capsids as solubility, as well as crystallization rates, are dependent on charge density^{37,48,78-84}. The PEG and NaCl concentrations and pH provide “levers' for influencing the charged interactions between the full capsids, and between the empty capsids. Changing the pH, which has a more direct effect on the charge density, has a stronger effect on the content ratio than changing the PEG concentration (FIG. 3). NaCl influences the charge environment around a full or empty capsid by controlling the electrical double layer thickness, just as for proteins and other types of colloidal particles⁸⁵⁻⁸⁹. As such, NaCl concentration controls the shielding of the inherent charge density of the capsids and hence the rate of their crystallization, just as for individual protein molecules^{48,79,81,83,84,90,91}. Together, pH and NaCl concentration may directly control the charge environment at the surface of individual capsids, which would directly affect capsid-to-capsid interactions and hence the solubility and crystallization rates.

For all pH values, the crystallization regions for full capsids span higher values of NaCl and PEG concentrations than for empty capsids (FIGS. 9 and 10). Although ionic interactions decrease rapidly as the distance between the ions increases, the capsid net charge and localized surface charge affect capsid-capsid interactions. The total, net, particle charge is due to the protein and nucleic acid in the full capsids. Although DNA is net negatively charged, protein charge results from amino acid composition and pI. The negatively charged areas at the surface of the full capsids would attract positively charged areas on other full capsids, creating more likelihood of attractions between full capsids that come in close proximity, and more propensity for crystal nucleation and more ordered arrangement of capsids. At higher PEG concentration, the solubility is lower and supersaturation is higher, which would encourage the formation of precipitants rather than crystals. A counteracting effect is that the crystal nucleation kinetics and orderliness of arrangement for full capsids would be expected to be faster due to the charge effects, which would favor the formation of crystals rather than precipitants. These competing effects enable crystallization of full capsids to occur at higher PEG concentrations than for empty capsids.

The sensitivity of the crystallization regions on pH is stronger for “empty” than for “full” capsids for some values of pH (FIGS. 4A-7B). A potential cause for this difference in dependency is that the isoelectronic point of 6.3 for empty capsids is clearly in the center of the pH range in the experiments, and of 5.9 for full capsids is near the edge of the pH range. The largest change in charge distribution occurs when the pH crosses the isoelectronic point, which is fully spanned for the empty capsids. This observation is consistent with the largest change in the charge distribution for empty capsids being associated with the largest change in the attractive forces between capsids, and thus the largest effect of the crystallization regions.

Capsid crystallization occurs at values of pH that are in the vicinity of the isoelectric point. The solubility is minimum for pH near the isoelectric point the electrostatic repulsion between proteins is the lowest when the proteins are at net zero charge⁹²⁻⁹⁴. The same result holds for other molecules in solution, and would also hold for capsids. For empty capsids (FIGS. 5B, 7B), crystallization at higher pH is enabled by using higher NaCl concentration. This observation is consistent with the higher ionic strength associated with higher NaCl concentration screening out some of the average repulsive charges of the capsids, so that individual capsids are more prone to come together by interacting at surfaces that are oppositely charged locally.

Although the shape of the crystallization regions is qualitatively similar for both the serotypes for both full and empty capsids, dependence of “empty” rAAV5 (FIG. 5B) on pH is observably different from others (FIGS. 4B, 6B, 7B).

For all capsids, for pH increasing from 5.7 to pI (which is 5.9 for full capsids and 6.3 for empty capsids), there is an increase in the size (i.e., shift) of the crystallization region towards relatively higher NaCl concentration. This is because decreasing solubility with increasing pH within this pH range needs higher ionic strength to maintain a supersaturation conducive to crystallization. However, on further increasing pH from pI to 7.2, crystallization region for all capsids except “empty” rAAV5 shifts to relatively lower NaCl concentration. This is because for all the capsids except “empty” rAAV5, crystallization region reached the ionic strength close to maximum solubility (˜2 M NaCl; ref) and thus on further increasing pH to 7.2 shifts the crystallization region to relatively lower NaCl and higher PEG. On the contrary, for “empty” rAAV5, the NaCl concentration used has not reached the solubility limit, which allows further addition of NaCl. However, this change in the size of the crystallization region is much more significant for empty capsids than full capsids.

The crystallization plots in FIGS. 4A-4B, 5A-5B, 6A-6B and 7A-7B identify the regions/conditions that favor the crystallization of only full capsids, only empty capsids, or both, and hence to understand the possibility of selectively separating either full or empty capsids from a mixture of both at different crystallization conditions. The crystallization regions for “full” and “empty” capsids for rAAV5 are shown in the same plot in FIG. 9, and for rAAV9 in FIG. 10. The pH in these experiments were chosen to be near the isoelectric point of full and empty rAAV capsids, that is, where the net charge on the surface is zero and the capsids have low solubility (close to that point) such as for other proteins⁹⁵⁻⁹⁸. The experiments varied the ionic strength of the droplet by adding NaCl at different molar concentrations. Regions where “full” and “empty” AAVs can be preferentially crystallized are clearly observed in FIGS. 9 and 10. These regions depend on pH and PEG and NaCl concentrations. For both rAAV5 and rAAV9, higher PEG concentration favours the preferential crystallization for “full” capsid and lower PEG favours the preferential crystallization of “empty” capsid. There is a common region where both full and empty capsids crystallize.

The region over which “full” rAAV5 is preferentially crystallized is widest at low pH (5.7) and the region in which preferential crystallization of “full” rAAV5 occurs is a factor of three smaller at high pH (7.2, FIG. 9). The preferential crystallization region for “empty” rAAV5 depends weakly on pH.

For rAAV9, the region for preferential crystallization of “full” capsid is widest at pH 6.3 (FIG. 10). At pH 7.2, “full” rAAV9 capsids can only be crystallized over a narrow region, which is more than a factor of 3 smaller than that at 6.3. The narrowest region for preferential crystallization of “empty” capsid occurs for high pH (7.2). The preferential crystallization region for both “full” and “empty” rAAV9 capsids strongly depends on pH.

As mentioned previously, the inherent charge distribution on full capsids favours crystallization at higher PEG concentration, while the empty capsid crystallization region is limited to low PEG because similar charges on the empty capsid surface allows crystallization at low PEG only. This allows the preferential crystallization of full capsids at higher PEG and of empty capsids at lower PEG at low pH. But, at high pH (7.2), higher solubility built the supersaturation conducive for crystallization at higher PEG and higher NaCl/ionic strength allows empty capsid crystal nucleation by screening the higher surface charge density on empty capsids. Therefore, at higher pH 7.2, crystallization regions for empty capsids almost overlap with that of full capsids and full capsids can only be crystallized over a narrow region.

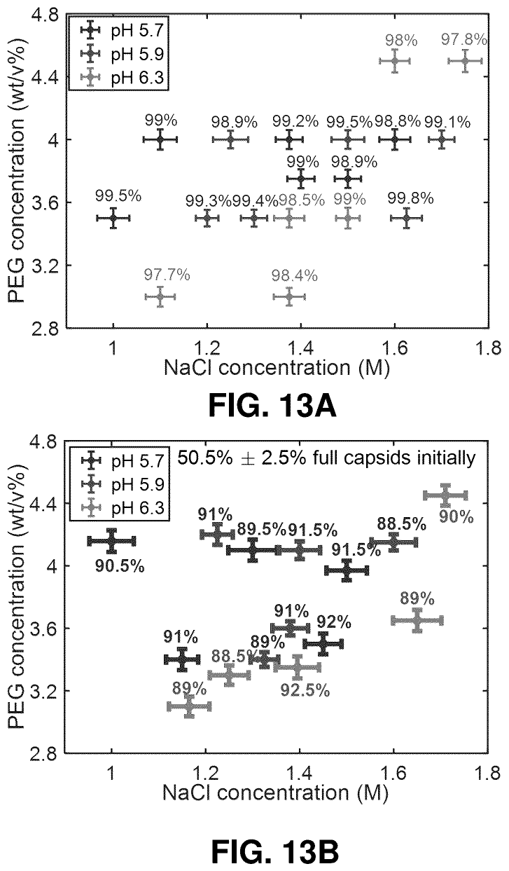

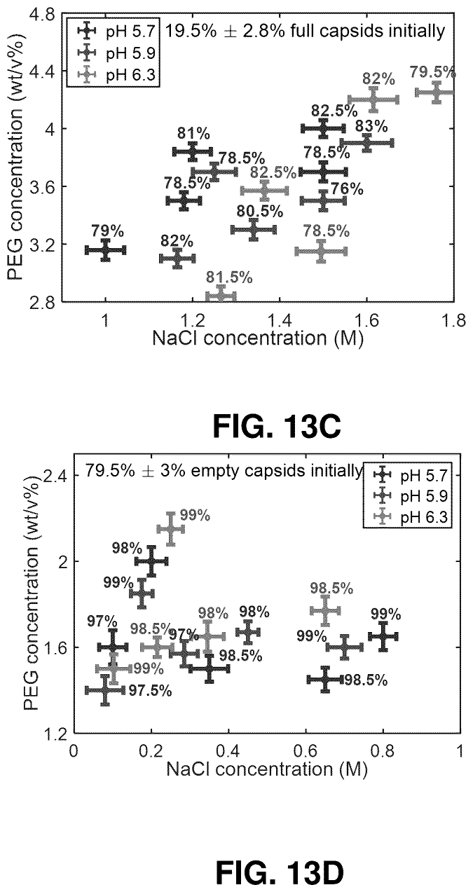

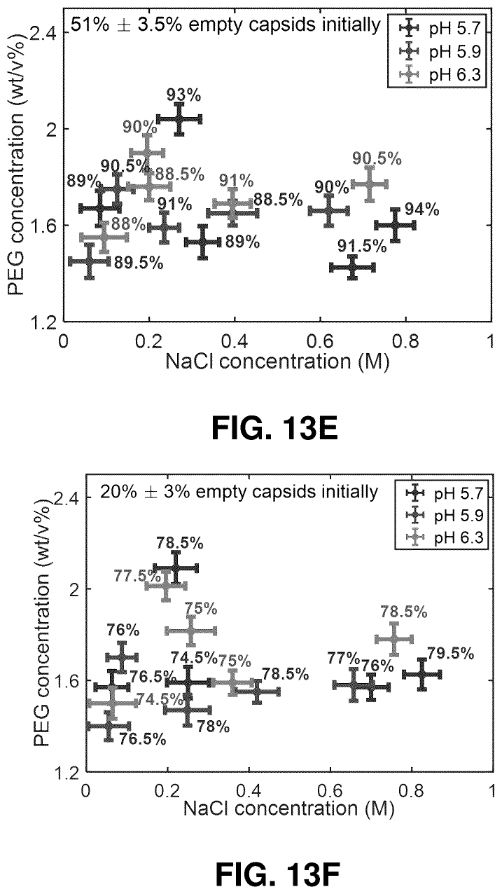

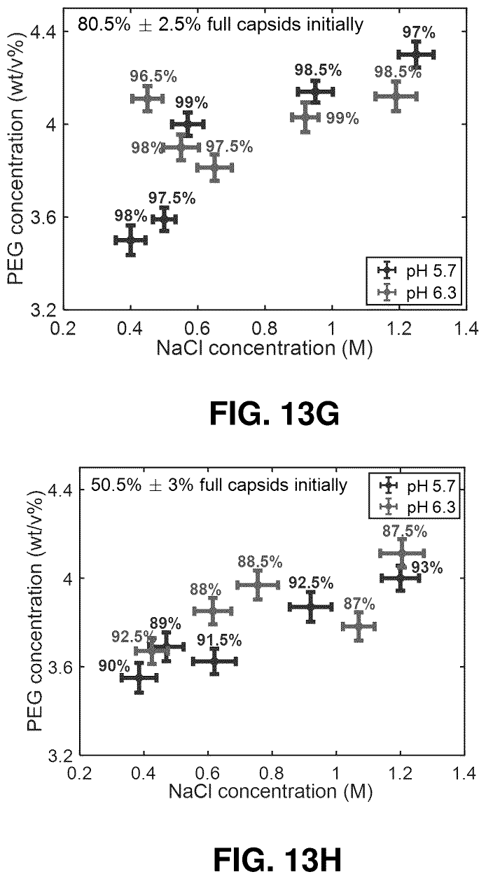

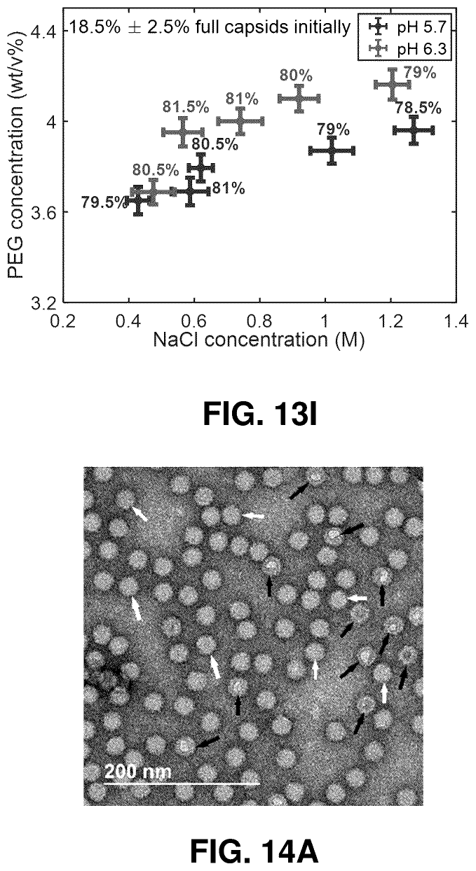

To understand the enrichment of full or empty capsids in the crystals after preferential crystallization, ddPCR and ELISA experiments were performed on dissolved capsid crystals, and the ratio of full and empty capsids was calculated. ELISA and ddPCR experiments are described in detail in Experimental Methods section. FIGS. 13A-C show the experimental condition at which preferential crystallization experiments for full rAAV5 were carried out and the corresponding full capsid percentage in the crystals. It shows that preferential crystallization of full capsids results in higher fraction of full capsids in crystals irrespective of the fraction of full capsids in the starting sample. It is significant that starting with a full capsid fraction of about 20%, preferential crystallization of full capsids can enrich to >80%. To obtain even higher fraction of full capsids, the starting fraction of full capsids in the sample must be higher. For example, a starting fraction of about 80% results in >95% full capsids in the crystals. Likewise, preferential crystallization of empty capsids (FIGS. 13D-13F) results in crystals enriched in empty capsids and an enrichment of up to 78% can be achieved starting with a sample having about 20% empty capsids. Similar analysis was performed for rAAV9 crystals (FIGS. 13G-13I). Here also preferential crystallization produces crystals enriched in full capsid to 78% full capsids from a sample having about 19% full capsids. Thus, crystallization in the preferential region of full capsids preferentially removed full capsids from the liquid solution and in the preferential region of empty capsids preferentially removed empty capsids from the solution.

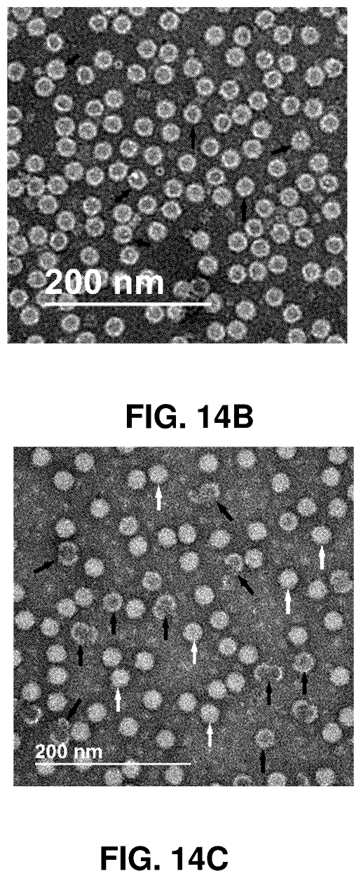

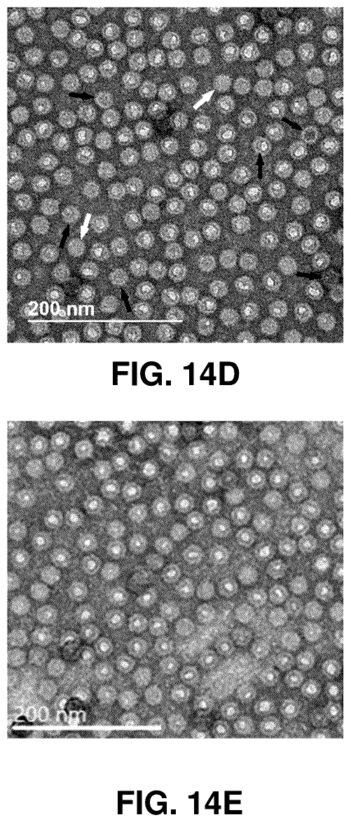

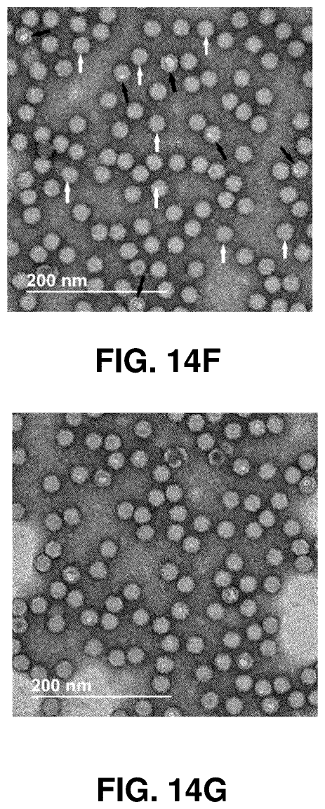

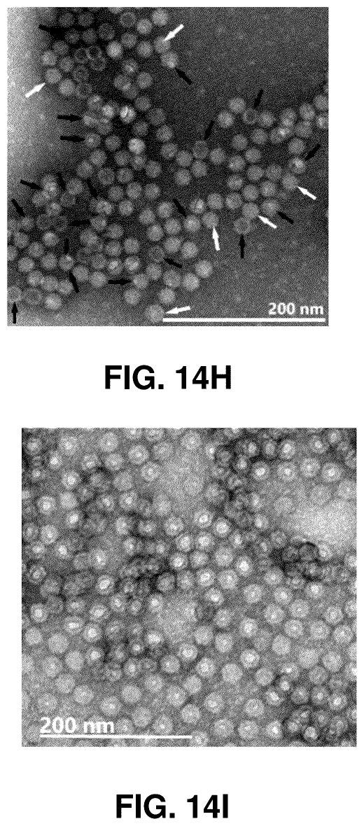

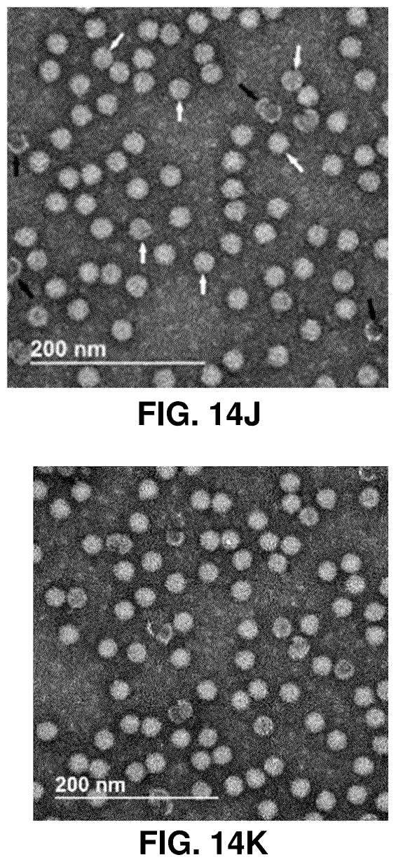

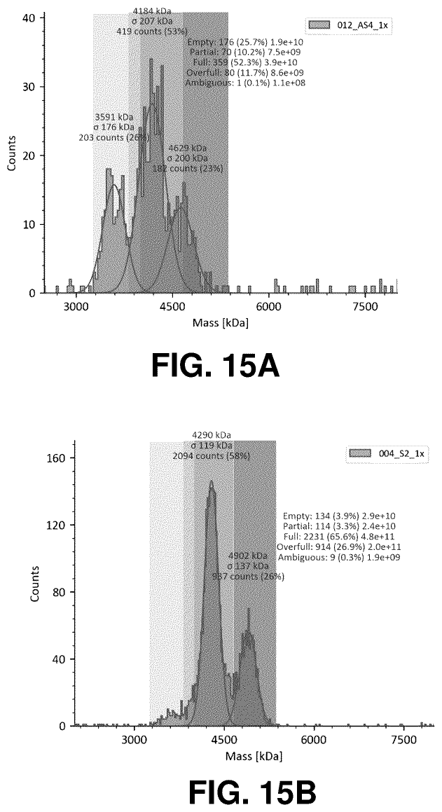

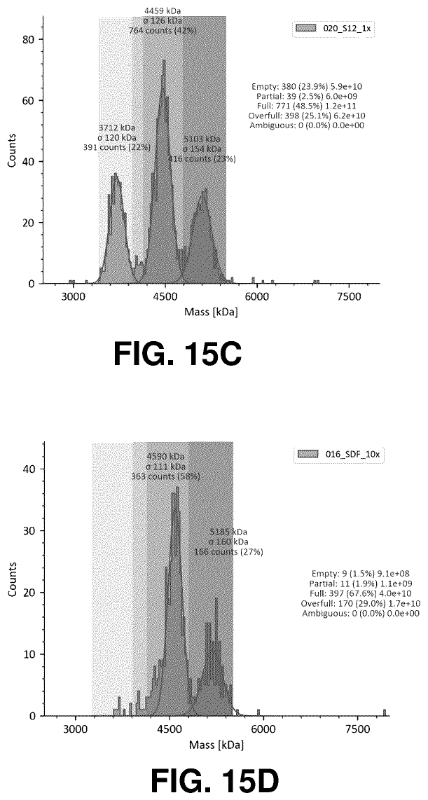

To investigate the morphology of capsid particles after crystallization, crystals were dissolved in PBS buffer for TEM imaging. TEM grids with capsid particles were prepared and images obtained and compared with the TEM image of capsid particles in standard sample purchased from Virovek. This is shown in FIGS. 14A-14L. A detailed description of sample preparation method and imaging technique is given in the experimental section. The heavy metal stain is taken up by the empty capsids, which diffracts the electron beam appearing as black areas (FIGS. 14A-L, black arrows) in the image, whereas full capsids exclude the heavy metal atoms and appear as uniformly shaded hexagons (FIGS. 14A-L, white arrows). The TEM image of the crystallization-purified sample suggests that capsid particles are homogeneous in shape and size with a diameter of ˜ 22-25 nm. Comparison of FIGS. 14E-14L with the reference sample (FIGS. 14A-14D) shows that the capsid's remains the same even after crystallization, which is true for both the serotypes. The percentage of full and empty capsids after preferential crystallization were calculated based on a total of ˜250 capsid particles from multiple TEM images (as one image e.g., as shown in FIG. 14F contains only ˜80-90 particles). This analysis suggests that, for both the serotypes, the full capsid's preferential crystallization-purified sample (FIG. 14F and FIG. 14J) contains >85% full capsids as compared to 20-25% full capsids in the starting sample (FIG. 14E and FIG. 14I). Likewise, for both the serotypes, empty capsid's preferential crystallization results in >90% empty capsids (FIGS. 14H and 14I) from 20-25% empty capsids in the starting sample (FIGS. 14G and 14K). Thus, TEM image analysis also supports the full-empty percentage calculated based on ddPCR and ELISA experiments. Mass photometry was performed to corroborate the ELISA, ddPCR, and TEM results of the samples before and after preferential crystallization of full capsids. FIGS. 15A-D show the corresponding results with full, empty, partially filled, and overfilled capsids percentage. It shows that for rAAV5, a starting sample with full+overfilled capsids of ˜64% (FIG. 15A) is enriched to about 93% full (FIG. 15B) and for rAAV9 (FIG. 15C), a starting sample that is ˜74% full is enriched to about 97% full (FIG. 15D). This further supports the TEM, ddPCR and ELISA results showing the full capsids enrichment.

In order to understand the effect of crystallization on biological activity, full rAAV5 and full rAAV9 were dissolved and used to transduce HEK293T cells and analyzed for reporter gene, GFP, expression (data not shown). After transduction, cells were observed under fluorescence microscope every 24 h for five days and transduction efficiency was measured on day 5 in Countess II automated cell counter. In almost all cases, weak GFP fluorescence appeared after 24 h post-transduction reaching maximum fluorescent intensity at day 5. As expected, GFP fluorescence was proportional to the MOI of either rAAV5 and rAAV9. For example, for rAAV5 crystal sample (data not shown), MOI of 10⁶ gives an transduction efficiency (i.e., % GFP positive cells) of 86%, compared to that of 75% for MOI 10⁵. However, 10× difference in MOI did not result in a similar 10-fold transduction efficiency indicating that MOISs of 1E+5 and 1E+6 were approaching the transduction limit, e.g., receptor saturation. Transduction efficiency of both the serotypes is close to each other. In case of both the serotypes, rAAV5 and rAAV9, transduction efficiencies for crystal sample agrees well with that for reference vectors (positive control) from Virovek. This suggests that biological activity and stability of the full rAAV capsids remain preserved in the crystals.

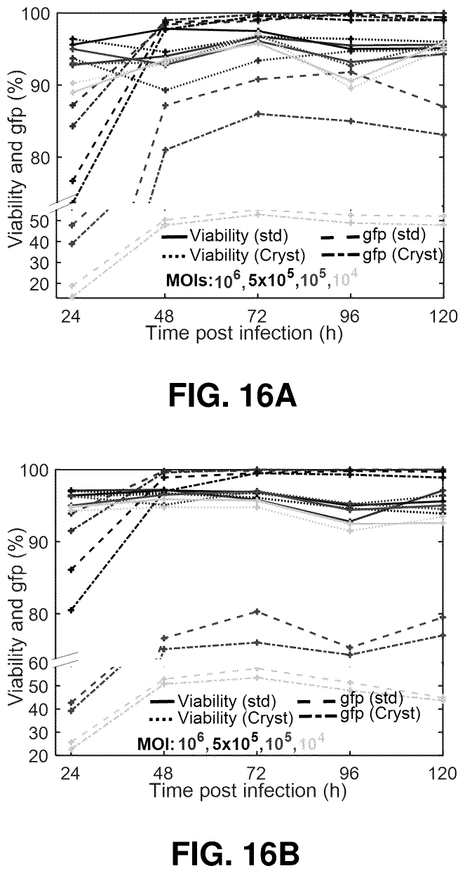

In adherent cell culture method, it is difficult to measure cell viability and infection efficiency in regular time intervals as it involves periodic detachment and washing of cells, which involves significant loss of cells. Therefore, flow cytometry analysis was performed to track the cell viability and transduction efficiency at regular time intervals as well as to obtain accurate measurement of viable and GFP-positive cells based on singlet cells population/statistics. The measurement was performed every 24 h for 5 days. FIGS. 16A-16B shows the corresponding results for rAAV5 and rAAV9. FIGS. 16A-16B show that for both the serotypes, rAAV5 and rAAV9, viability and % GFP-positive cells in the crystalized sample is comparable to that of the positive control (standard) for all MOIs throughout the five days (data not shown). The difference in viability and GFP-positive cells between positive control and experimental samples is not significant. In all cases, cell viability remains above 90% throughout the experiment ensuring that all the statistics are based on high population of live cells. It was observed that, for a MOI of 10⁶, >85% of viable cells are transduced within 24 h, while only ˜15% of viable cells are transduced in the same period of time when the virus dosage is reduced by an order of 2 (i.e., MOI 10⁴) (see SI) and the infection rate is so slow that only 40-50% of viable cells are transduced after day 5 (FIGS. 16A-16B). The percentage GFP-positive cells measured in the countess II automatic cell counter is low as compared to that in flow cytometry result. This is probably due to instrumental accuracy. Thus, the flow cytometry results also suggests that the biological activity of capsids remains preserved after crystallization. TCID₅₀ experiment was performed to quantify the “infectious” virus titre. For experimental procedure, please visit the experimental section. For rAAV5 crystal stock solution, value of infectious dosage was found to be 2.5×10⁴ TCID50/0.1 mL in five days as compared to 1.5×10⁴ TCID50/0.1 mL in five days in the standard rAAV5 sample before crystallization. Similarly, for rAAV9 crystal sample, value of infectious dosage was found to 1.3×10⁴TCID50/0.1 mL in five days as compared to 7.8×10³ TCID50/0.1 mL in five days in the standard rAAV9 sample before crystallization. Thus, there is no significant difference in dosage required between the standard and the crystallized stock solution. This suggests that the biological activity of full capsids remains preserved even after crystallization.

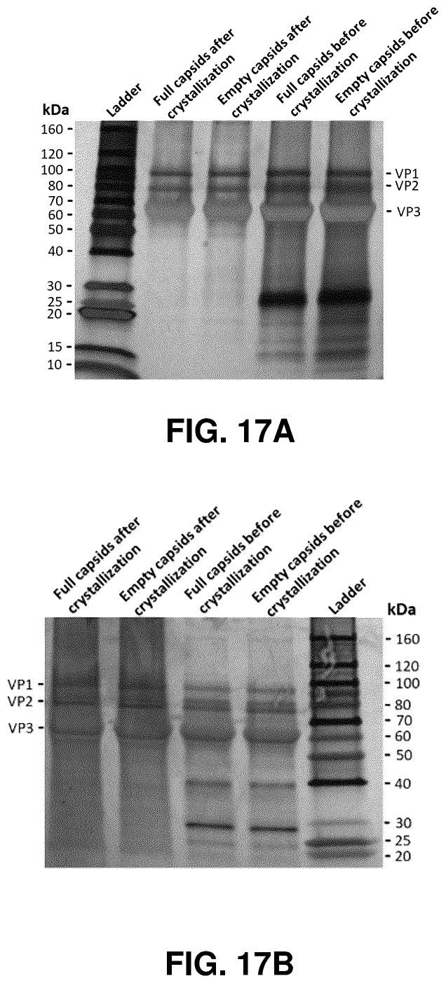

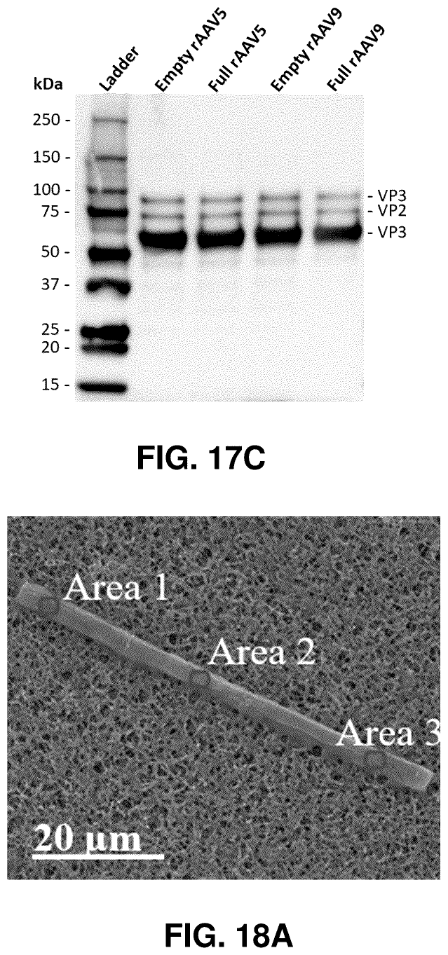

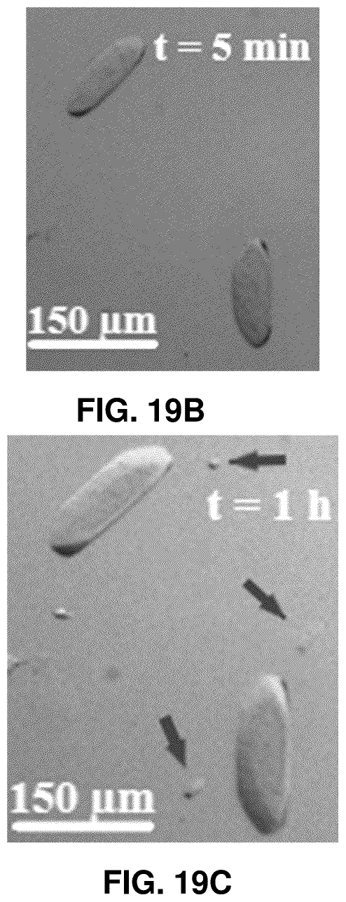

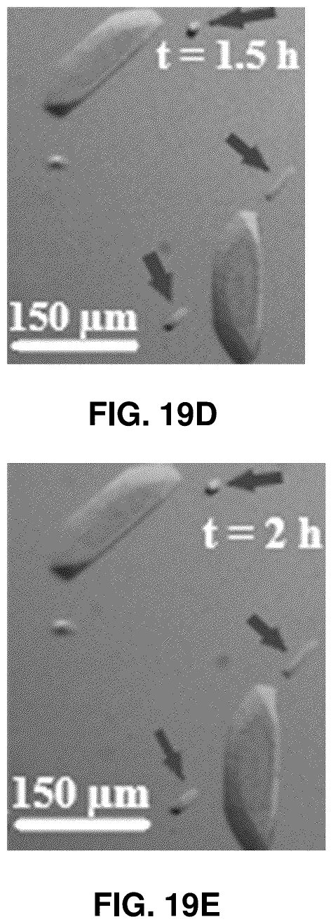

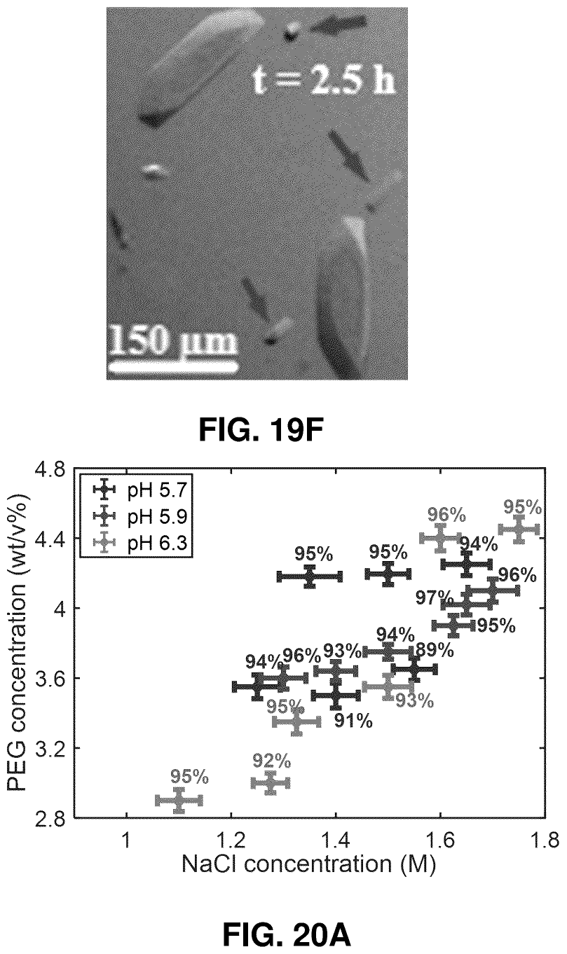

SDS-PAGE gel electrophoresis was performed to assess vector purity and determine integrity of capsid proteins following crystallization. FIGS. 17A and 17B show the corresponding electrophoresis results for rAAV5 and rAAV9 and for both full and empty capsids before and after crystallization. These images confirm the presence of all three VP proteins VP1, VP2, and VP3 in crystallized capsids and the quantification of proteins suggests that the ratio of VPs in the crystallized sample remains almost the same as that before crystallization. The image also suggests that the crystallization process removes other low molecular weight impurities present in the rAAV samples obtained from Virovek. To rule the possibility that the prominent bands at 28 kDa and other bands are not from capsid proteins, western blot experiment was performed. The corresponding western blot results are shown in FIG. 19C for rAAV5 and rAAV9. Western blot results show that the bands below 50 kDa are not from capsid proteins and represents impurities from rAAV sample.

All the previous screenings of the crystallization conditions used PEG8000 and NaCl as precipitants. To understand whether the method of preferential crystallization works with other precipitants, crystallization conditions were screened using PEG6000 and NaCl (FIG. 12), as well as PEG8000 and MgCl₂ (FIG. 13A) as precipitants. In both cases, it is found that “full” and “empty” capsids can be preferentially crystallized from a mixture. Similar to the PEG8000 and NaCl combination (FIGS. 4A-7B), the qualitative shape of the phase diagram for “full” and “empty” capsids crystallized using both PEG6000 with NaCl and PEG8000 with MgCl2 remain almost the same. Similar to PEG8000 (FIG. 9), for PEG6000 (FIG. 12), the preferential crystallization region for “full” capsid lies at relatively higher PEG6000 and NaCl concentration, whereas the preferential crystallization region for “empty” capsid lies at relatively lower PEG6000 and NaCl concentrations. However, PEG6000 needs higher concentration of salt to build the supersaturation level favourable for crystallization. A similar observation holds true for MgCl₂, except for the fact that, as compared to the NaCl (FIG. 9), MgCl₂ needs relatively lower concentration to build the supersaturation level conducive for the crystallization. Therefore, in conclusion, preferential crystallization works well with PEG of any molecular weight and with inorganic salt of any valency.

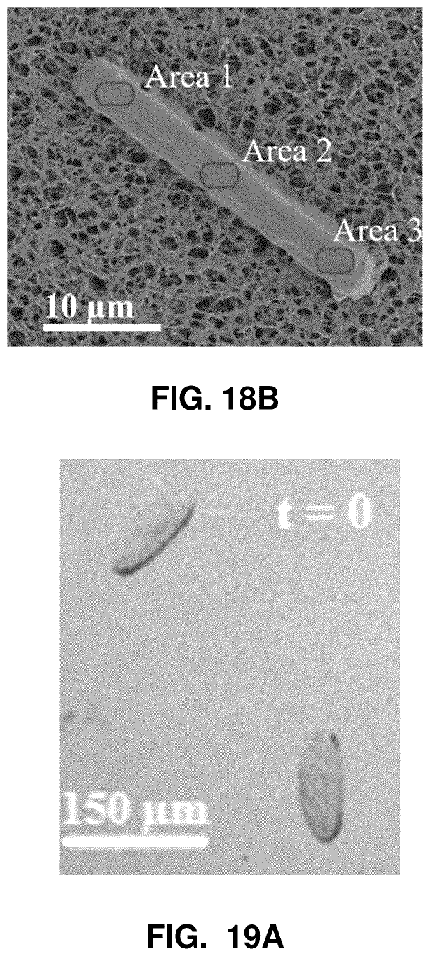

In order to understand the elemental composition of crystals, point EDAX (energy dispersive X-ray analysis) analysis was performed at different locations on rAAV crystals. FIGS. 18A and 18B show the image of locations at which EDAX analysis was performed on representative crystals of rAAV5 and rAAV9 and the table 1 shows the corresponding elemental compositions. For elemental mapping and line EDAX analysis, readers are requested to visit supplemental information section (SI). EDAX suggests that the major element in the crystal is carbon (atomic %>75) followed by oxygen (atomic %>9) and nitrogen (atomic %>7). This is consistent as carbon, and hydrogen are the two major constituent elements in any protein, followed by oxygen and nitrogen, and sulfur. Peptide chains are a major source of all four elements. Due to low mass, EDAX does not detect hydrogen. The presence of a very small fraction of Na, K, and Cl (atomic %<1) in crystals indicates that crystal purity is high and that crystalline NaCl is excluded. Table 1 also indicates that for both the serotypes, the elemental composition of crystals is similar at different locations.

As discussed above, the crystallization process in hanging drop vapor diffusion system may require 1-2 weeks for crystal nucleation followed by 1-2 weeks for crystal growth (depending on the crystal size). This is because supersaturation of the droplet occurs as a result of water evaporation and thus, the timing of primary nucleation is determined by the evaporation. In industrial crystallization, seed crystals are used to induce/promote secondary nucleation and growth. This reduces the time necessary for nucleation and the growth phase dominates the crystallization process. Seeding experiments were performed to understand the time required for the seed crystals/new crystals to grow to a separable/filtrable/realistic size (minimum ˜10 μm).⁹⁹ Detailed description of the seeding experiment was given in experiment section. FIGS. 19A-19F show that crystals start growing within minutes after the addition of capsid and precipitant mixture into the droplet.

Initially the growth rate was faster due to the presence of more capsids in the solution phase. After a few hours, the growth rate slows down and crystals eventually stop growing. FIGS. 19A-19F show that all the seed crystals grow by ˜50 μm within 1-1.5 h after the addition of capsids and precipitant mix into the droplet.

This suggests that the addition of seed crystals into a crystallization system will reduce the overall crystallization time from 2-3 weeks to <2-3 h, which is comparable to the time required for purification of 150 mL of cell supernatant by density gradient ultracentrifugation, 54 mL of cell supernatant by affinity chromatography, and 830 mL of cell supernatant by steric exclusion chromatography.¹⁰⁰ However, the latter two methods need additional purification steps afterwards such as ion exchange chromatography, and/or dialysis, while the density gradient ultracentrifugation needs affinity chromatography purified material as a feed, which increases the processing time further. Thus, most of the purification methods are inherently limited by binding capacity, regeneration efficiency, inability to separate full-empty capsids, yield/recovery, and non-scalability. On the other hand, preferential crystallization has no such limitation making it a suitable potential alternative.

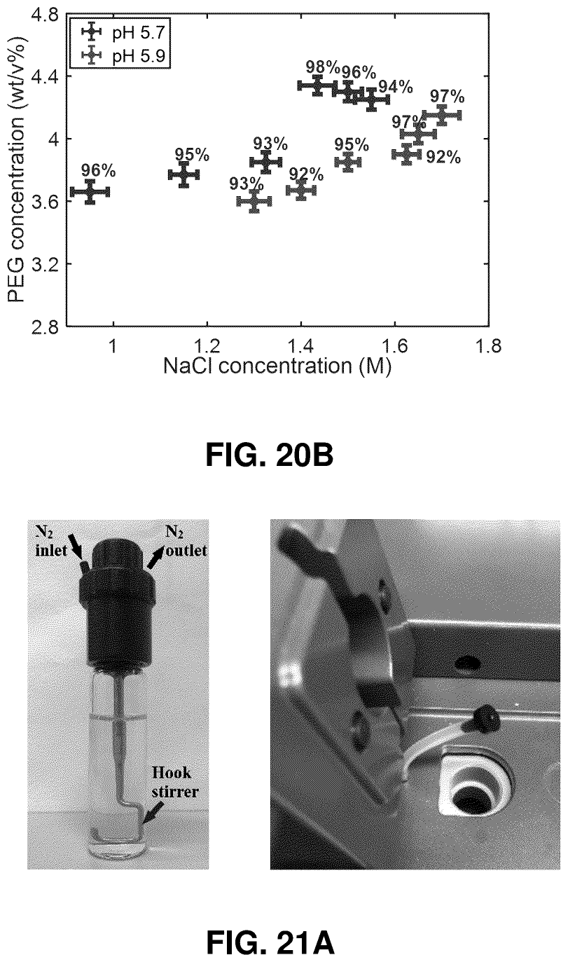

At the end, yield of the crystallization process (i.e., percentage of the initial capsids that formed crystals) was calculated. A detailed description of the yield calculation procedure was described in the experiment section. All the crystallization experiments were carried out with a starting capsid concentration of 10¹⁴ vg/mL. It is observed in the FIGS. 20A-20B that for both the serotypes, rAAV5 (FIG. 20A) and rAAV9 (FIG. 20B), the yield varies with precipitant and salt concentration with no distinct trend. In most of the crystallization conditions evaluated, the yield is >90%. This is because the solubility of protein varies with precipitant as well as salt concentration. The crystallization region moves towards the low concentration of rAAV as the precipitant concentration increases, which favours the crystallization of more capsids from the solution as the supersaturation is higher. The yield obtained in a crystallization process is much higher than that obtained in a chromatography process such as single cycle anion exchange chromatography (AEX-70%) or in density gradient ultracentrifugation-based purification method (<20%) and is comparable to the steric exclusion chromatography method (95%) and affinity chromatography (95%).^{100,101}

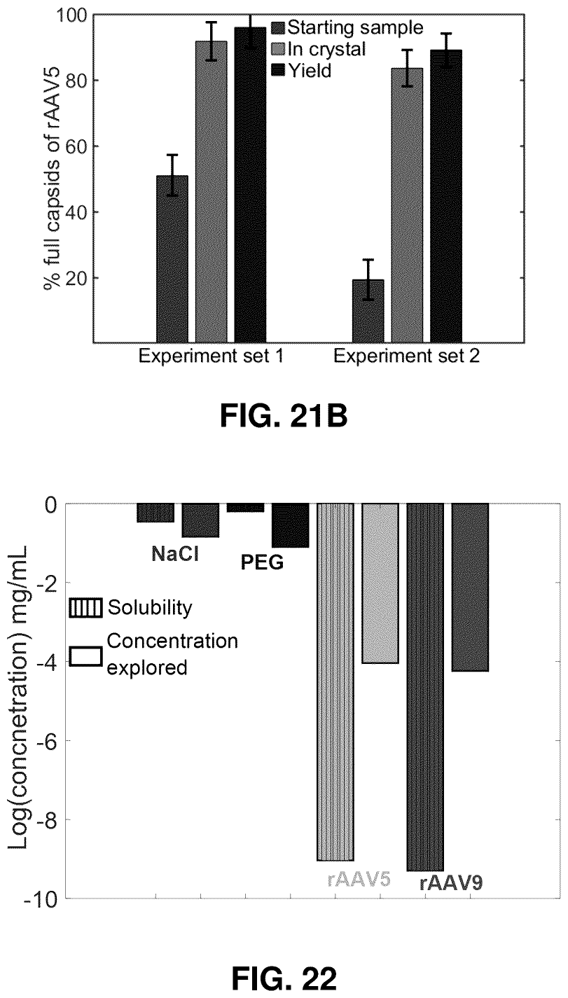

In order to understand whether the method of preferential crystallization works in a large-scale crystallizer, crystallization experiments were performed in a 5 mL crystallizer chamber. A detailed description of scale up experiment is given in the experiment section. FIG. 21B shows experimental results for rAAV5 for starting capsids solution with 51%, and 19.4% full capsids. It is observed, % of full capsids (i.e., purity) in the crystals is close to that found in the hanging drop experiment (FIG. 14B) for all the starting capsids solutions. Yields obtained in the scaled-up experiment is also in good agreement with the droplet experimental results. These scale-up results suggest that the preferential crystallization observed in μL volume droplet experiments are reproducible in a larger scale, mL volume reactor and supports the conclusion that the preferential crystallization is scalable. Due to the unavailability of large sample quantities, the scaled-up experiment could not be performed in crystallizer volume greater than 7 mL, but as the experimental results suggest, the conditions used for preferential crystallization experiment in droplet or the 7 mL crystallizer can be used to perform preferential crystallization in mL or L scale crystallizers without further modification as the thermodynamics of crystallization remains the same across scales. Thus, both the percentage of full capsids and yield achieved in preferential crystallization method presented in this work is better than that achieved in one round of conventional CEX (cation exchange chromatography)-AEX (80% full, 63% yield). AEX alone is only capable of a 70% yield from a starting vector composition of almost 15% full and 80% empty capsids.¹⁰¹ However, because of the absence of literature data on full-empty capsids ratio after CEX, it has not been possible to compare directly the full capsid enrichment data with that occurred during AEX alone. Density gradient ultracentrifugation may approach approximately 90% full capsid purity, but the recovery is extremely low, often <20%. Sequential isopycnic gradients have been utilized to achieve higher full capsid enrichment, but at a substantial loss of recovered vector. Scale up results suggests that starting with a low percentage of full capsids, two rounds of preferential crystallization can give a purity >95% with a recovery (yield) almost 80% considering 89% recovery in each round (FIGS. 21A-21B). In contrast, two rounds of AEX would result in a recovery of only 50% and two rounds of density gradient ultracentrifugation would give a recovery <10%,^{100,101} Thus, the preferential crystallization presented in this work has the potential to be used in the downstream purification and enrichment of full rAAV capsids from a mixture of full and empty capsids.

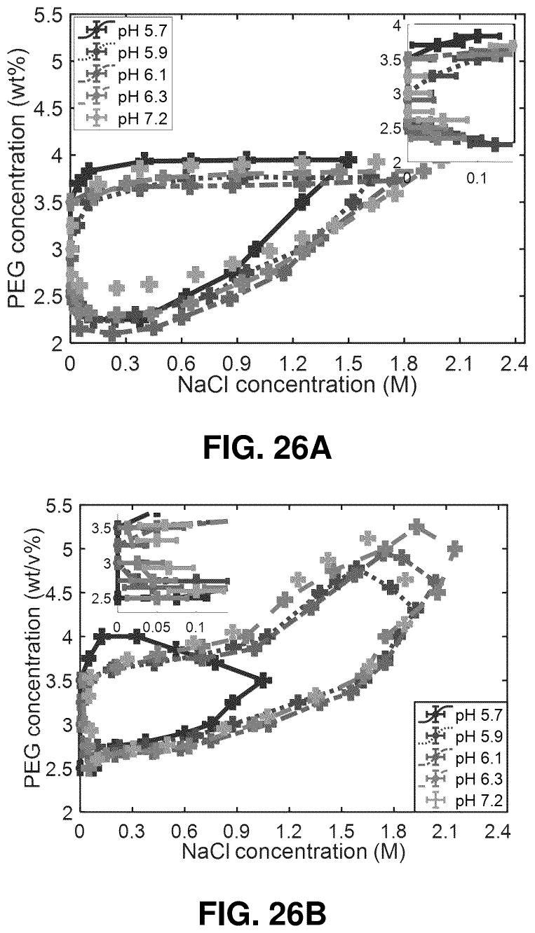

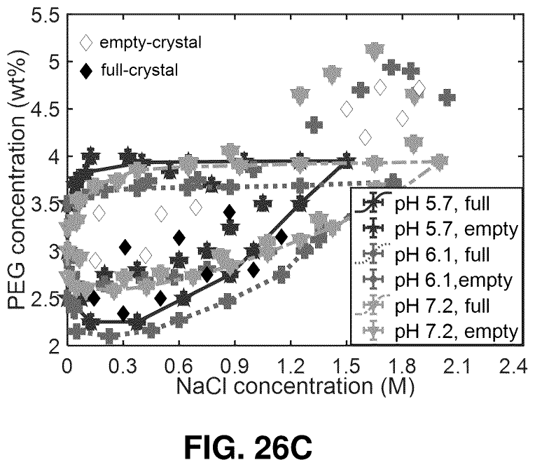

A similar phase diagram analysis was performed for rAAV8 to find the preferential crystallization region, which is shown in FIGS. 26A-26C. This further corroborates previous results for rAAV5 and rAAV9 suggesting the presence of preferential crystallization region for full and empty capsids for all rAAV serotypes, which can be used to purify full capsids or empty capsids from their mixture.

### SI.1: Solubility Analysis

Solubility is an important parameter in crystallization process as solubility determines the supersaturation level. Component with low solubility has higher probability of forming nuclei as compared to the component with higher solubility. Thus, comparison of solubilities of different components with their concentrations used in the crystallization process (FIG. 22) can give an idea of component forming crystals and this helps is ruling out the possibility that any of the particles formed in this study consist of NaCl or PEG. The explored concentrations of NaCl of 0.01 to 4 M are much lower (FIG. 22) than the solubility of NaCl (˜6M) in aqueous solution at room temperature.^{1,2} Similarly, the concentrations of PEG explored in this article of 1 to 7 wt/v % are much lower (FIG. 22) than the solubility of PEG (˜0.63 g/ml, sigmaaldrich.com) in aqueous solution at room temperature. On the other hand, minimum concentrations of both rAAV5 and rAAV8 used in this analysis remains well above their solubility limits (FIG. 22). This suggests that the particles formed in this study consists of rAAV capsids and are not from either NaCl or PEG.

### SI.2: Phase Diagram Construction

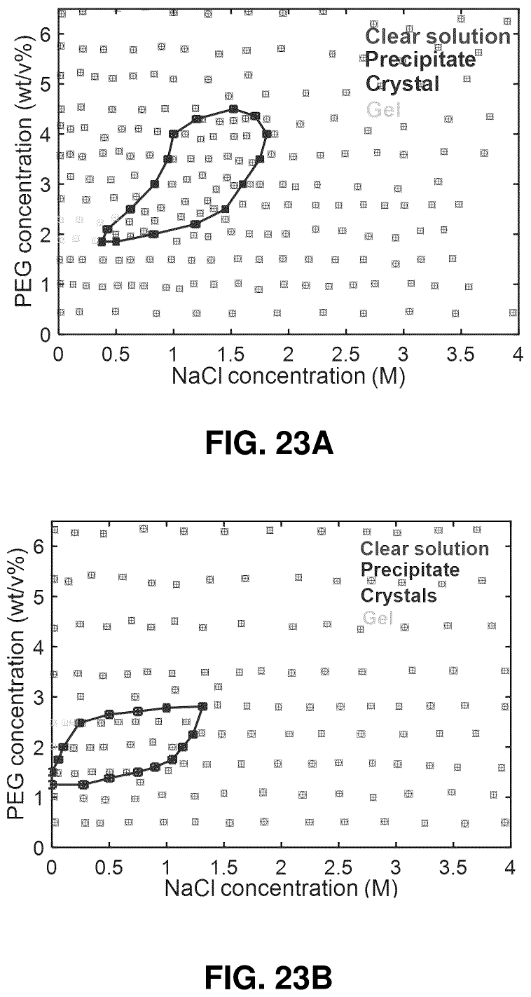

Crystallization regions for rAAV5 and rAAV9 capsids were not reported in literature except for a few crystallization conditions for “empty” capsids of rAAV5 and rAAV9.^{3,4} In order to identify preferential crystallization regions, it's extremely important to understand the crystallization behavior of both full and empty capsids as a function of precipitant concentration. To identify the region favorable for crystallization of capsids, hanging drop vapor diffusion experiments were carried out and crystallization conditions were screened over a wide range of NaCl, PEG, and pH. There is no ideal way to select screening conditions. Crystallization parameter space can be thought of consisted of discretized points, each of which represents an experimental (screening) condition and is separated from each other by certain values of parameters. For example, sample phase diagrams are shown in FIGS. 23A-23B for full and empty capsids for both the serotypes at pH 5.7. Each phase diagram consisted of experimental data points as represented by colored symbols with error bars. For each pH, screening of experimental conditions was started from low concentration of precipitants and then precipitants concentration was gradually increased. Selection of an experimental condition (i.e., the increment of precipitant concentration) was based on the outcomes from the experiment in the previous experimental conditions (i.e., previous data points) Each experiment was tracked for a period of a couple of weeks and experimental outcome is noted down. Prominent outcomes of the screening experiments were formation of crystals, precipitate, clear solution, gel spots, and/or unidentified particles of different shapes, which are shown by experimental data points of different color. For each pH, similar phase diagrams were prepared this way. Significantly, for both the serotypes and for both full and empty capsids, crystallization occurs only over a relatively narrow range of precipitant and pH concentrations.

### SI.2: SAED Analysis of Particles

XRD analysis is most commonly used method to confirm the crystallinity of a material (Xie, Q. et al. Proc Natl Acad Sci USA 99, 10405-10410 (2002); Stevenson, H. P. et al. Acta Crystallogr D Struct Biol 72, 603-615 (2016); Stevenson, H. P. et al. Proc Natl Acad Sci USA 111, 8470-8475 (2014)). XRD requires a large amount of sample (˜0.2 g), so instead selected area electron diffraction (SAED) analysis in TEM is used to confirm the crystallinity (Stevenson, H. P. et al. Acta Crystallogr D Struct Biol 72, 603-615 (2016); Stevenson, H. P. et al. Proc Natl Acad Sci USA 111, 8470-8475 (2014)). The presence of the spot diffraction patterns in the reciprocal lattice is strong evidence that rAAV capsid particles are crystals of capsids. The ring pattern in the reciprocal lattice for “empty” rAAV8 crystals indicates that some of the particles are polycrystalline. The lack of a ring for the other capsids indicates that the particles are single crystals (Asadabad, M. and Eskandari, M. 3-25 (Intechopen, 2016); Cowley, J. M. Prog Mater Sci 13, 267-321 (1968)).

In a polarizer, a non-polarized white light (infinite directions of vibration) is passed through a polarizer fixed at its position and a plane polarized light is produced.⁵ Next, the plane polarized light enters the crystal mounted on the microscope stage. If the crystal is anisotropic, plane polarized light is split into two rays, one fast-moving ray and another slow-moving ray due to the direction dependent speed of the light through the crystal. These two rays are vibrating perpendicular to each other, but parallel to the crystallographic axis. The polarized light waves then travel through the analyzer (which can be rotated against the polarizer by 360°). Analyzer combines these two rays and allows to pass only those rays, which are parallel to the analyzer and thus parallel to each other. Since one wave is retarded with respect to the other, either constructive or destructive interference occurs between the split waves as they pass through the analyzer. As a result, the birefringent crystals acquire a spectrum of interference color when observed in white light through crossed polarizers.⁵⁻⁷ By rotating the analyzer at different angles, one could view different interference colors (data not shown). By making a quantitative analysis of the interference color with the help of Michel-Levy chart, one can estimate the order of birefringence, thickness of the sample, path difference, and one can get a rough idea of electronic/structural environment inside the crystals, which have been already discussed in the main article.⁷

### SI.5: TEM Images of Capsid Particles

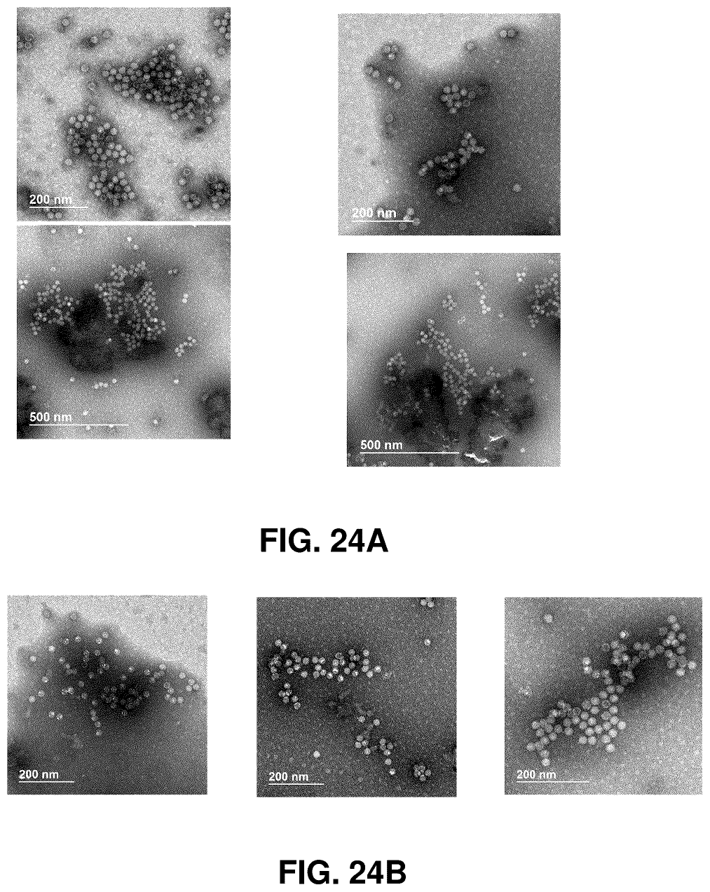

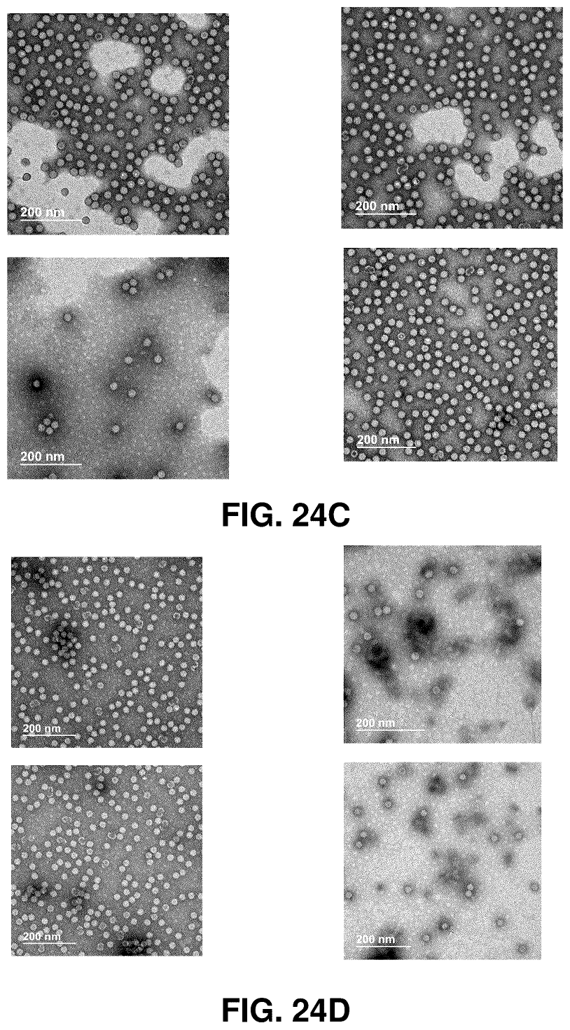

Capsid particles were observed under TEM to understand the morphology as well as the relative amount of full and empty capsids.⁸ To endure the accuracy of the analysis, for each sample, TEM images were captured at different locations of the sample from repeated runs to obtain large number of particles. As an example, FIGS. 24A-24D shows some of the TEM images of capsids obtained after preferential crystallization of full and empty capsids for both the serotypes. Images suggest that capsids obtained after preferential crystallization is rich in either full or empty capsids depending on preferential crystallization region. Thus, crystallization of capsids in preferential crystallization region selectively removes the target capsids from the solution containing both full and empty capsids. Images suggest that empty capsids have higher tendency to stay close to each other and probably form aggregate, while full capsids tend to stay dispersed in the solution. As expected, presence of negatively charged DNA in full capsids prevents itself from aggregation.

### SI.6: SEM Images of Ordered Layer of Capsids Particles at Different Locations of Crystals

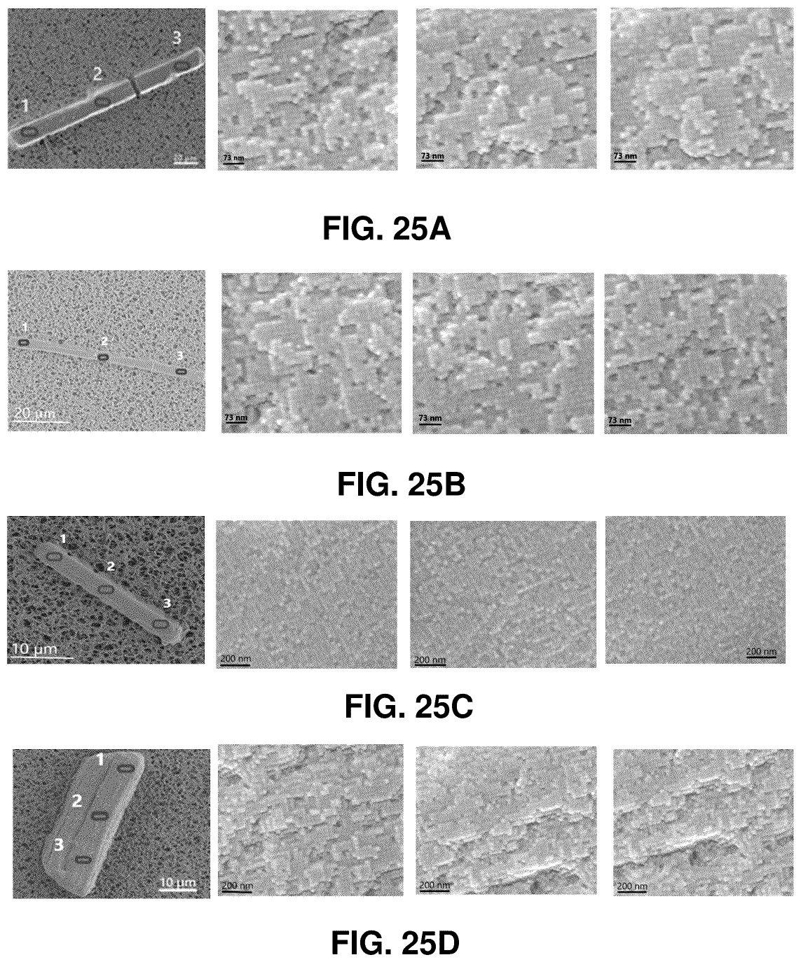

Crystals were observed under SEM to understand the surface feature of the crystals. FIGS. 25A-25D shows the corresponding SEM images for crystals of both serotypes. It shows that crystals are composed of small spherical particles, which are organized in orderly manner. This pattern is repeated in the images taken at different location on the same crystal. Formation of multiple layers of capsids on the top of each other is a characteristic of crystal growth phase, where growth may be by attachment of single capsids on the kink site or by 2D surface nucleation followed by attachment growth. Multiple small islets of ordered capsids on the tope of wider layer are suggestive of formation of 2D nuclei, whereas presence of isolated capsids near the end of a layer are suggestive of attachment growth at kink sites.

### SI.7: EDAX Analysis of Crystals

EDAX analysis was performed to understand the elemental composition of crystal. Before performing elemental analysis of each crystal, elemental mapping was performed on the region of membrane holding the crystal (data not shown). Analysis suggested that carbon is the only element, which is present in both crystal as well as membrane surface. Fraction of nitrogen and oxygen in the crystal is much more than that in neighboring membrane region. Likewise, fraction of sodium, chlorine, sulfur and phosphorous seems to be little higher in crystals than that in the membrane. As expected, the fraction of fluorine is almost zero in crystals. Table 1 shows the composition obtained from mapping analysis.

Line analysis was performed on the crystal to obtain actual elemental composition of crystals. In this analysis, each analysis was made along the length of crystal. This analysis was made at three different locations across the crystal. Table 1 shows the elemental composition of crystal after line analysis. Crystal is primarily composed of carbon, nitrogen, oxygen and the fraction of sodium and chlorine is very low.

TABLE 1
Elemental composition obtained from EDAX analysis

| | | Atomic |
|---|---|---|
| | Atomic % | % from line |
| | from area | mapping on |
| Element | mapping | crystal |
| C K | 76.57 | 75.08 |
| N K | 4.05 | 9.63 |
| O K | 10.20 | 11.66 |
| F K | 2.03 | 0.09 |
| Na K | 1.40 | 0.41 |
| P K | 3.01 | 1.61 |
| S K | 2.39 | 1.05 |
| Cl K | 0.39 | 0.25 |
| K K | 0.01 | 0.01 |

### S.11. Concentration of Virovek AAV Sample

The procedure for concentration of AAV samples involved multiple steps. First an ultrafiltration filter was washed by adding 500 μL of buffer solution and centrifuging at 14,000 g for 5 minutes. The filtrate and retentate were discarded. The filter was then used immediately or stored immersed in a buffer solution at 4° C. for up to a week. To concentrate the sample, 450 μL of buffer solution and 40 μL of sample were added to the filter and mixed by aspirating and dispensing repeatedly. Then the ultrafiltration filter was weighed and assembled with a microcentrifuge tube. The tube was then centrifuged at 14,000 g until less than 20 μL retentate remains. The ultrafiltration filter was then weighed again and 450 μL of buffer was added and centrifuged again at 14,000 g until less than 20 μL retentate remains. The filtrate tube was weighed again and reassembled in the reverse direction with a new microcentrifuge tube. The filtrate was recovered by centrifugation at 1,000 g for 2 min. Both the tube with recovered retentate and filter were weighed again. This procedure yields around 20 μL of buffer solution with concentration increased by a factor of two.

### CONCLUSION

There is range of NaCl and pH over which preferential crystallization of full rAAVs is possible. By precisely controlling the pH and NaCl concentration in that regime, one can effectively separate out the full rAAVs from a cell lysate solution. Thus, this preferential crystallization can be an effective alternative to the existing technology in the downstream processing of the AAVs. A model based on PBE presented herein, can serve as an effective tool to extract kinetic parameters and predict the crystal characteristics, thereby helping in scale up and design of the industrial-scale crystallizer.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

The invention can be further understood in view of the following numbered paragraphs.
1. A method of purifying full capsids of recombinant adeno-associated virus vectors (rAAVs) containing recombinant therapeutic genes from a mixture of full rAAV capsids and empty rAAV capsids having no recombinant therapeutic genes, comprising
   (i) mixing the mixture with a crystallization solution, wherein the crystallization solution comprising a pre-determined set of conditions of precipitant concentration, salt concentration, and pH, wherein the pre-determined set of conditions of precipitant concentration, salt concentration, and pH preferentially crystalize the full rAAV capsids over the empty rAAV capsids;
   (ii) providing an effective amount of time to allow crystals of the full rAAV capsids to grow; and
   (iii) removing the crystals of the full rAAV capsids from the mixture, thereby purifying the full rAAV capsids from the mixture.
2. The method of paragraph 1, wherein the initial capsid concentration of the mixture of full rAAV capsids and the empty rAAV capsids is between about 0.1×10¹⁴ and about 5×10¹⁴ vg/ml, inclusive, or between about 0.25×10¹⁴ and about 2.5×10¹⁴ vg/ml, inclusive.
3. The method of paragraph 1 or 2, wherein the mixture of full rAAV capsids and empty rAAV capsids comprises about 50%, 60%, 70%, 80%, 90%, or more than 90% full rAAV capsids out of the total rAAV capsids.
4. The method of any one of paragraphs 1-3, wherein prior to step (i) the method comprises a step of obtaining the crystallization region to provide a phase diagram for each of the full rAAV capsids and the empty rAAV capsids by varying one or more parameters of precipitant concentration, salt concentration, and pH; and a further step of superimposing the phase diagram for the full rAAV capsids and the phase diagram for the empty rAAV capsids to identify the range of precipitant concentration, salt concentration, and pH that preferentially crystalizes the full rAAV capsids over empty rAAV capsids to provide the pre-determined set of conditions.
5. The method of any one of paragraphs 1-4, wherein the precipitant is polyethylene glycol (PEG).
6. The method of any one of paragraphs 1-5, wherein the precipitant is PEG8000 or PEG6000.
7. The method of any one of paragraphs 1-6, wherein the precipitant concentration is between about 0.5 and about 12 w/v %, inclusive.
8. The method of any one of paragraphs 1-7, wherein the salt is sodium chloride or magnesium chloride.
9. The method of any one of paragraphs 1-8, wherein the salt concentration is between about 0.01 to 4.5 M, inclusive.
10. The method of any one of paragraphs 1-9, wherein the pH range is between about 5.5 and about 7.5, inclusive.
11. The method of any one of paragraphs 1-10, wherein the rAAVs are of a serotype selected from the group consisting of AAV1, AAV2, AAV3, AAV 3B, AAV4, AAV5, AAV6, AAV7, AAV8, AAV9, AVV10, AAV11, AAV12, BAAV, AAAV, or AAV VR-942. 12. The method of any one of paragraphs 1-11, wherein the rAAVs are of a serotype AAV5, AAV8, or AAV9.
13. The method of paragraph 12, wherein the rAAVs are of serotype AAV5, and wherein the pre-determined set of conditions comprise PEG8000 as the precipitant with a concentration between about 1.8 and about 8 w/v %, inclusive; sodium chloride as the salt with a concentration between about 0.3 and about 2.0 M, inclusive; and a pH between about 5.5 and about 7.2, inclusive.
14. The method of paragraph 12, wherein the rAAVs are of serotype AAV5, and wherein the pre-determined set of conditions comprise PEG8000 as the precipitant with a concentration between about 2.8 and about 4.3 w/v %, inclusive; sodium chloride as the salt with a concentration between about 0.8 and about 1.8 M, inclusive; and a pH between about 5.6 and about 6.8, inclusive.
15. The method of paragraph 12, wherein the rAAVs are of serotype AAV5, and wherein the pre-determined set of conditions comprise PEG8000 as the precipitant with a concentration between about 2.0 and about 4.0 w/v %, inclusive; sodium chloride as the salt with a concentration between about 1 and about 1.5 M, inclusive; and a pH between about 5.0 and about 7.2, inclusive.
16. The method of paragraph 12, wherein the rAAVs are of serotype AAV9, and wherein the pre-determined set of conditions comprise PEG8000 as the precipitant with a concentration between about 2.0 and about 3.5 w/v %, inclusive; sodium chloride as the salt with a concentration between about 0.01 and about 1.2 M, inclusive; and a pH between about 5.6 and about 6.9, inclusive.
17. The method of paragraph 12, wherein the rAAVs are of serotype AAV5, and wherein the pre-determined set of conditions comprise PEG6000 as the precipitant with a concentration between about 1.8 and about 8.0 w/v %, inclusive; sodium chloride as the salt with a concentration between about 0.01 and about 2.2 M, inclusive; and a pH between about 5.5 and about 7.4, inclusive.
18. The method of paragraph 12, wherein the rAAVs are of serotype AAV5, and wherein the pre-determined set of conditions comprise PEG6000 as the precipitant with a concentration between about 2.5 and about 5.4 w/v %, inclusive; sodium chloride as the salt with a concentration between about 0.7 and about 1.9 M, inclusive; and a pH between about 5.6 and about 6.9, inclusive.
19. The method of paragraph 12, wherein the rAAVs are of serotype AAV5, and wherein the pre-determined set of conditions comprise PEG8000 as the precipitant with a concentration between about 1.7 and about 8.0 w/v %, inclusive; magnesium chloride as the salt with a concentration between about 0.01 and about 1.8 M, inclusive; and a pH between about 5.5 and about 7.4, inclusive.
20. The method of paragraph 12, wherein the rAAVs are of serotype AAV5, and wherein the pre-determined set of conditions comprise PEG8000 as the precipitant with a concentration between about 2.6 and about 5.6 w/v %, inclusive; magnesium chloride as the salt with a concentration between about 0.3 and about 1.6 M, inclusive; and a pH between about 5.6 and about 6.9, inclusive.
21. The method of paragraph 12, wherein the rAAVs are of serotype AAV9, and wherein the pre-determined set of conditions comprise PEG8000 as the precipitant with a concentration between about 2.0 and about 7.8 w/v %, inclusive; sodium chloride as the salt with a concentration between about 0.01 and about 2.3 M, inclusive; and a pH between about 5.5 and about 7.4, inclusive.
22. The method of paragraph 12, wherein the rAAVs are of serotype AAV9, and wherein the pre-determined set of conditions comprise PEG8000 as the precipitant with a concentration between about 3.5 and about 4.7 w/v %, inclusive; sodium chloride as the salt with a concentration between about 0.2 and about 1.5 M, inclusive; and a pH between about 5.6 and about 6.9, inclusive.
23. The method of any one of paragraphs 1-22, wherein the method comprises storing the crystals of the full rAAVs capsids after purification for short-term or long-term.
24. The method of paragraph 23, wherein the crystals are stored at between about 2° C. and about 20° C., inclusive, preferably between about 4° C. and about 10° C., inclusive.
25. The method of any one of paragraphs 1-24, wherein the method comprises adding one or more cryoprotectant to the crystals of the full rAAVs capsids and then storing the crystals at between about −1° C. and about −80° C., inclusive, preferably between about −10° C. and about −30° C., inclusive.
26. The method of any one of paragraphs 1-25, wherein the method comprises reconstituting the crystals of the full rAAVs capsids with a pharmaceutically acceptable excipient for administering to a subject in need of gene therapies.
27. The method of paragraph 26, wherein the subject is in need of a genetic enzyme replacement therapy or has one or more of Duchenne muscular dystrophy, limb girdle muscular dystrophy type 2D, Leber's hereditary optic neuropathy, late infantile neuronal ceroid lipofuscinosis, rheumatoid arthritis, mucopolysaccharidosis, spinal muscular atrophy, X-linked juvenile retinoschisis, Dysferlin deficiency, hemophilia A, hemophilia B, metachromatic leukodystrophy, idiopathic Parkinson's disease, and Alzheimer's disease.
28. A crystal of full capsids of recombinant adeno-associated virus vectors (rAAVs) containing recombinant therapeutic genes, wherein the crystal is prepared and purified according to the method of any one of claims 1-27.
29. The crystal of paragraph 28, wherein the crystal has minimal empty rAAVs capsids having no recombinant therapeutic genes, about less than 5%, 4%, 3%, 2%, or 1% out of total rAAVs capsids in the crystal.
30. A crystal of full capsids of recombinant adeno-associated virus vectors (rAAVs) of serotype AAV5 containing recombinant therapeutic genes, wherein the crystal is prepared by the steps of
   (i) mixing a mixture containing full rAAV5 capsids and empty rAAV5 capsids having no recombinant therapeutic genes with one or more crystallization solution, wherein the crystallization solution comprises PEG8000 as a precipitant with a concentration between about 1.8 and about 8.0 w/v %, inclusive; sodium chloride as the salt with a concentration between about 0.3 and about 2.0 M; and a pH between about 5.5 and about 7.2, inclusive; and
   (ii) providing an effective amount time to allow crystals of the full rAAV5 capsids to grow.
31. The crystal of paragraph 30, wherein the crystallization solution comprises PEG8000 as the precipitant with a concentration between about 2.8 and about 4.3 w/v %, inclusive; sodium chloride as the salt with a concentration between about 0.8 and about 1.8 M; and a pH between about 5.6 and about 6.8, inclusive.
32. A crystal of full capsids of recombinant adeno-associated virus vectors (rAAVs) of serotype AAV8 containing recombinant therapeutic genes, wherein the crystal is prepared by the steps of
   (i) mixing a mixture containing full rAAV8 capsids and empty rAAV8 capsids having no recombinant therapeutic genes with one or more crystallization solution, wherein the crystallization solution comprises PEG as a precipitant with a concentration between about 2.0 and about 4.0 w/v %, inclusive; sodium chloride as the salt with a concentration between about 0.01 and about 1.5 M; and a pH between about 5.5 and about 7.2, inclusive.
   (ii) providing an effective amount time to allow crystals of the full rAAV8 capsids to grow.
33. The crystal of paragraph 30, wherein the crystallization solution comprises PEG as the precipitant with a concentration between about 2.0 and about 2.6 w/v %, inclusive; sodium chloride as the salt with a concentration between about 0.01 and about 1.2 M; and a pH between about 5.6 and about 6.9, inclusive.
34. A crystal of full capsids of recombinant adeno-associated virus vectors (rAAVs) of serotype AAV5 containing recombinant therapeutic genes, wherein the crystal is prepared by the steps of
   (i) mixing a mixture containing full rAAV5 capsids and empty rAAV5 capsids having no recombinant therapeutic genes with one or more crystallization solution, wherein the crystallization solution comprises PEG6000 as the precipitant with a concentration between about 1.8 and about 8.0 w/v %, inclusive; sodium chloride as the salt with a concentration between about 0.01 and about 2.2 M, inclusive; and a pH between about 5.5 and about 7.4, inclusive; and
   (ii) providing an effective amount time to allow crystals of the full rAAV5 capsids to grow.
35. The crystal of paragraph 34, wherein the crystallization solution comprises PEG6000 as the precipitant with a concentration between about 2.5 and about 5.4 w/v %, inclusive; sodium chloride as the salt with a concentration between about 0.7 and about 1.9 M, inclusive; and a pH between about 5.6 and about 6.9, inclusive.
36. A crystal of full capsids of recombinant adeno-associated virus vectors (rAAVs) of serotype AAV5 containing recombinant therapeutic genes, wherein the crystal is prepared by the steps of
   (i) mixing a mixture containing full rAAV5 capsids and empty rAAV5 capsids having no recombinant therapeutic genes with one or more crystallization solution, wherein the crystallization solution comprises PEG8000 as the precipitant with a concentration between about 1.7 and about 8.0 w/v %, inclusive; magnesium chloride as the salt with a concentration between about 0.01 and about 1.8 M, inclusive; and a pH between about 5.5 and about 7.4, inclusive; and
   (ii) providing an effective amount time to allow crystals of the full rAAV5 capsids to grow.
37. The crystal of paragraph 36, wherein the crystallization solution comprisesPEG8000 as the precipitant with a concentration between about 2.6 and about 5.6 w/v %, inclusive; magnesium chloride as the salt with a concentration between about 0.3 and about 1.6 M, inclusive; and a pH between about 5.6 and about 6.9, inclusive
38. A crystal of full capsids of recombinant adeno-associated virus vectors (rAAVs) of serotype AAV9 containing recombinant therapeutic genes, wherein the crystal is prepared by the steps of
   (i) mixing a mixture containing full rAAV5 capsids and empty rAAV9 capsids having no recombinant therapeutic genes with one or more crystallization solution, wherein the crystallization solution comprises PEG8000 as the precipitant with a concentration between about 2.0 and about 7.8 w/v %, inclusive; sodium chloride as the salt with a concentration between about 0.01 and about 2.3 M, inclusive; and a pH between about 5.5 and about 7.4, inclusive; and
   (ii) providing an effective amount time to allow crystals of the full rAAV5 capsids to grow.
39. The crystal of paragraph 38, wherein the crystallization solution comprises PEG8000 as the precipitant with a concentration between about 3.5 and about 4.7 w/v %, inclusive; sodium chloride as the salt with a concentration between about 0.2 and about 1.5 M, inclusive; and a pH between about 5.6 and about 6.9, inclusive.
40. The crystal of any one of paragraphs 28-39, further comprising a step of removing the crystals of the full rAAV5 or rAAV8 capsids from the mixture, optionally storing the crystals for short-term or long-term.
41. A method of purifying empty capsids of recombinant adeno-associated virus vectors (rAAVs) containing no recombinant therapeutic genes from a mixture of empty rAAV capsids and full rAAV capsids having recombinant therapeutic genes, comprising
   (i) mixing the mixture with a crystallization solution, wherein the crystallization solution comprising a pre-determined set of conditions of precipitant concentration, salt concentration, and pH, wherein the pre-determined set of conditions of precipitant concentration, salt concentration, and pH preferentially crystalize the empty rAAV capsids over the full rAAV capsids;
   (ii) providing an effective amount of time to allow crystals of the full rAAV capsids to grow; and
   (iii) removing the crystals of the empty rAAV capsids from the mixture, thereby purifying empty capsids and enriching the full rAAV capsids in the solution of the mixture.
42. The method of paragraph 41, wherein the initial capsid concentration of the mixture of full rAAV capsids and the empty rAAV capsids is between about 0.1×10¹⁴ and about 5×10¹⁴ vg/ml, inclusive, or between about 0.25×10¹⁴ and about 2.5×10¹⁴ vg/ml, inclusive.
43. The method of paragraph 41 or 42, wherein the mixture of full rAAV capsids and empty rAAV capsids comprises about 50%, 60%, 70%, 80%, 90%, or more than 90% empty rAAV capsids out of the total rAAV capsids.
44. The method of any one of paragraphs 41-43, wherein prior to step (i) the method comprises a step of obtaining the crystallization region to provide a phase diagram for each of the full rAAV capsids and the empty rAAV capsids by varying one or more parameters of precipitant concentration, salt concentration, and pH; and a further step of superimposing the phase diagram for the full rAAV capsids and the phase diagram for the empty rAAV capsids to identify the range of precipitant concentration, salt concentration, and pH that preferentially crystalizes the empty rAAV capsids over full rAAV capsids to provide the pre-determined set of conditions.
45. The method of any one of paragraphs 41-44, wherein the precipitant is polyethylene glycol (PEG).
46. The method of any one of paragraphs 41-45, wherein the precipitant is PEG8000 or PEG6000.
47. The method of any one of paragraphs 41-46, wherein the precipitant concentration is between about 0.5 and about 12 w/v %, inclusive.
48. The method of any one of paragraphs 41-47, wherein the salt is sodium chloride or magnesium chloride.
49. The method of any one of paragraphs 41-48, wherein the salt concentration is between about 0.01 to 4.5 M, inclusive.
50. The method of any one of paragraphs41-49, wherein the pH range is between about 5.5 and about 7.5, inclusive.
51. The method of any one of paragraphs 41-50, wherein the rAAVs are of a serotype selected from the group consisting of AAV1, AAV2, AAV3, AAV 3B, AAV4, AAV5, AAV6, AAV7, AAV8, AAV9, AVV10, AAV11, AAV12, BAAV, AAAV, or AAV VR-942.
52. The method of any one of paragraphs 41-51, wherein the rAAVs are of a serotype AAV5, AAV8, or AAV9.
53. The method of paragraph 52, wherein the rAAVs are of serotype AAV5, and wherein the pre-determined set of conditions comprise PEG8000 as the precipitant with a concentration between about 0.8 and about 4 w/v %, inclusive; sodium chloride as the salt with a concentration between about 0.001 and about 1.4 M, inclusive; and a pH between about 5.5 and about 7.2, inclusive.
54. The method of paragraph 52, wherein the rAAVs are of serotype AAV5, and wherein the pre-determined set of conditions comprise PEG8000 as the precipitant with a concentration between about 0.9 and about 2.5 w/v %, inclusive; sodium chloride as the salt with a concentration between about 0.001 and about 1.2 M, inclusive; and a pH between about 5.6 and about 6.9, inclusive.
55. The method of claim 52, wherein the rAAVs are of serotype AAV8, and wherein the pre-determined set of conditions comprise PEG8000 as the precipitant with a concentration between about 2.3 and about 5.5 w/v %, inclusive; sodium chloride as the salt with a concentration between about 0.001 and about 2.4 M, inclusive; and a pH between about 5.5 and about 7.2, inclusive.
56. The method of paragraph 52, wherein the rAAVs are of serotype AAV8, and wherein the pre-determined set of conditions comprise PEG8000 as the precipitant with a concentration between about 3 and about 5 w/v %, inclusive; sodium chloride as the salt with a concentration between about 1.3 and about 2.1 M, inclusive; and a pH between about 5.6 and about 6.9, inclusive.
57. The method of paragraph 52, wherein the rAAVs are of serotype AAV5, and wherein the pre-determined set of conditions comprise PEG6000 as the precipitant with a concentration between about 1.5 and about 8 w/v %, inclusive; sodium chloride as the salt with a concentration between about 0.01 and about 2 M, inclusive; and a pH between about 5.5 and about 7.4, inclusive.
58. The method of paragraph 52, wherein the rAAVs are of serotype AAV5, and wherein the pre-determined set of conditions comprise PEG6000 as the precipitant with a concentration between about 2.1 and about 3.5 w/v %, inclusive; sodium chloride as the salt with a concentration between about 0.01 and about 0.8 M, inclusive; and a pH between about 5.6 and about 6.9, inclusive.
59. The method of paragraph 52, wherein the rAAVs are of serotype AAV5, and wherein the pre-determined set of conditions comprise PEG8000 as the precipitant with a concentration between about 1.5 and about 7.5 w/v %, inclusive; magnesium chloride as the salt with a concentration between about 0.001 and about 1.5 M, inclusive; and a pH between about 5.5 and about 7.4, inclusive.
60. The method of paragraph 52, wherein the rAAVs are of serotype AAV5, and wherein the pre-determined set of conditions comprise PEG8000 as the precipitant with a concentration between about 1.8 and about 3.4 w/v %, inclusive; magnesium chloride as the salt with a concentration between about 0.001 and about 0.8 M, inclusive; and a pH between about 5.6 and about 6.9, inclusive.
61. The method of paragraph 52, wherein the rAAVs are of serotype AAV9, and wherein the pre-determined set of conditions comprise PEG8000 as the precipitant with a concentration between about 1.8 and about 7.5 w/v %, inclusive; sodium chloride as the salt with a concentration between about 0.01 and about 2.3 M, inclusive; and a pH between about 5.5 and about 7.4, inclusive.
62. The method of paragraph 52, wherein the rAAVs are of serotype AAV9, and wherein the pre-determined set of conditions comprise PEG8000 as the precipitant with a concentration between about 2.3 and about 3.5 w/v %, inclusive; sodium chloride as the salt with a concentration between about 0.01 and about 1.5 M, inclusive; and a pH between about 5.6 and about 7.4, inclusive.

### REFERENCES

1. Valdmanis, et al. Hum Gene Ther 28, 361-372 (2017).
2. Ma, et al. Biotechnol Adv 40, 107502 (2020).
3. Ginn, et al. Journal of Gene Medicine 20, 1-16 (2018).
4. Dunbar, et al. Science (1979) 359, 1-3 (2018).
5. Fattah, et al. Proc Natl Acad Sci USA 105, 8703-8708 (2008).
6. Schnödt, et al. Hum Gene Ther Methods 28, 101-108 (2017).
7. Gao, Ket al. Mol Ther Methods Clin Dev 1, 9 (2014).
8. Timmers, et al. Hum Gene Ther 31, 80-89 (2020).
9. Guillou, et al. Blood Adv. 6, 4266-4270 (2022).
10. Venkatakrishnan, et al. J Virol 87, 4974-4984 (2013).
11. Senapathy et al. Journal of Biological Chemistry 259, 4661-4666 (1984).
12. Khatwani, S et al. Mol Ther Methods Clin Dev 21, 548-558 (2021).
13. Nass, et al. Mol Ther Methods Clin Dev 9, 33-46 (2018).
14. Chen, et al. Molecular Therapy 24, S42 (2016).
15. Yu, et al. Mol Ther Methods Clin Dev 17, 34-48 (2020).
16. Qu, et al. Curr Pharm Biotechnol 16, 684-695 (2015).
17. Xie,. et al. Proc Natl Acad Sci USA 99, 10405-10410 (2002).
18. Lerch, et al. Acta Crystallogr Sect F Struct Biol Cryst Commun 65, 177-183 (2009).
19. Xie, et al. J Virol Methods 122, 17-27 (2004).
20. Xie, et al. Acta Crystallogr Sect F Struct Biol Cryst Commun 64, 1074-1078 (2008).
21. Miller, et al. Acta Crystallogr Sect F Struct Biol Cryst Commun 62, 1271-1274 (2006).
22. Nam, et al. J Virol 81, 12260-12271 (2007).
23. Mitchell, et al. Acta Crystallogr Sect F Struct Biol Cryst Commun 65, 715-718 (2009).
24. Nam, et al. J Virol 85, 11791-11799 (2011).
25. Mikals, et al. 186, 308-317 (2014).
26. Kaludov, Virology 306, 1-6 (2003).
27. Urabe, Hum Gene Ther. 13, 1935-1943 (2002).
28. Sylvain Cecchini, Hum Gene Ther. 22, 1021-1030 (2011).
29. Gruntman, et al. Hum Gene Ther Methods 26, 71-76 (2015).
30. Croyle, Gene Ther 8, 1281-1290 (2001).
31. Lins-Austin, et al. Viruses 12, 1-18 (2020).
32. Stevenson, et al. Acta Crystallogr D Struct Biol 72, 603-615 (2016).
33. Davidson, et al. J Appl Phys 18, 499-511 (1947).
34. Stevenson, et al. Proc Natl Acad Sci USA 111, 8470-8475 (2014).
35. Rieser, et al. J Pharm Sci 111, 2288-2298 (2022).
36. Lei, et al. Virologica Sinica 36, 141-144 (2021).
37. Bonneté, et al. Cryst Growth Des 7, 2176-2181 (2007).
38. Charles, et al. Acta Crystallogr D Biol Crystallogr 62, 1311-1318 (2006).
39. Webei, et al. Cryst Growth Des 8, 711-716 (2008).
40. McGarvey, et al. Tenside, Surfactants, Detergents 55, 203-209 (2018).
41. Soleymani, et al. J Mol Liq 316, 113777 (2020).
42. Heeb, et al. ACS Appl Mater Interfaces 1, 1105-1112 (2009).
43. Brunchi, et al. Revue Roumaine de Chimie 58, 183-188 (2013).
44. Sommer, et al. Molecular Therapy 7, 122-128 (2003).
45. Nwokeoji, et al. Anal Chem 89, 13567-13574 (2017).
46. Gerstein, A. S. Molecular Biology Problem Solver: A Laboratory Guide. (John Wiley & Sons, Inc., New York, 2001).
47. Gill, et al. Anal Biochem 182, 319-326 (1989).
48. Yamanaka,. et al. J Synchrotron Radiat 18, 84-87 (2011).
49. Hekmat, Bioprocess Biosyst Eng 38, 1209-1231 (2015).
50. Nesse, Introduction to Optical Minerology. (Oxford University Press, New York, NY, 2004).
51. Bloss, Crystallography and Crystal Chemistry. (Hold, Rinehart, Winston, Inc., New York, NY, 2012).
52. Dyar, et al. Mineralogy and Optical Mineralogy. (Mineralogical Society of America, Chantilly, V A, 2008).
53. Polowsky, et al. J Dairy Sci 101, 7714-7723 (2018).
54. Cowley, Prog Mater Sci 13, 267-321 (1968).
55. Asadabad, et al. Electron diffraction. in Modern electron microscopy in physical and life sciences (eds. Janecek, M. & Karl, R.) 3-25 (Intechopen, London, U K, 2016).
56. Idrobo, et al. Appl Phys Lett 95, 164101 (2009).
57. Zayas, JFunctionality of Proteins in Food. (Springer-Verlag, Berlin, 1997).
58. Ozbas et al. Macromolecules 37, 7331-7337 (2004).
59. Asherie, Methods 34, 266-272 (2004).
60. Broide, et al. Proc Natl Acad Sci USA 88, 5660-5664 (1991).
61. Ziegler et al. Adv Food Nutr Res 34, 203-298 (1990).
62. Trilisky et al. Biotechnol Prog 27, 1054-1067 (2011).
63. Martin, et al. Nat Commun 12, (2021).
64. Pegram, et al. Proc Natl Acad Sci USA 107, 7716-7721 (2010).
65. McPherson, et al. Acta Crystallographica Section F: Structural Biology Communications 70, 2-20 (2014).
66. Arawaka, et al. Advances in Comparative and Environmental Physiology: Volume and Osmolality Control in Animal Cells. (Springer-Verlag, Heidelberg, 1991).
67. Dumetz, et al. Protein Science 16, 1867-1877 (2007).
68. Curtis, R. A. & Lue, L. A molecular approach to bioseparations: Protein-protein and protein-salt interactions. Chem Eng Sci 61, 907-923 (2006).
69. Hass, et al. Relation between the solubility of proteins in aqueous solutions and the second virial coefficient of the solution. J. Phys. Chem. B 103 (14) 2808-2811 (1999).
70. Quigley, et al. European Journal of Pharmaceutics and Biopharmaceutics 96, 282-290 (2015).
71. Mehta, et al. Biotechnol Prog 28, 163-170 (2012).
72. Arakawa, et al. Methods Enzymol. 114, 49-77 (1985).
73. Zhang, et al. J. Pharm. Sci. 101, 965-977 (2012).
74. Nam, et al. J Virol 81, 12260-12271 (2007).
75. Nam, et al. J Virol 85, 11791-11799 (2011).
76. Halder, S et al. J Struct Biol. 192, 21-36 (2015).
77. Randolph, A. D.; Larson, M. A. Theory of Particulate Processes. (Elsevier, Amsterdam, 1971).
78. McPherson, et al. Acta Crystallographica Section F: Structural Biology Communications 70, 2-20 (2014).
79. Schmit, et al. J Am Chem Soc 134, 3934-3937 (2012).
80. Kuzmanic, et al. Biophys J 106, 677-686 (2014).
81. Warren, J. Phys. Cond. Mat. 14, 7617 (2002).
82. Yin, et al. J. Phys. Chem. C 115, 10357-10364 (2011).
83. Li, et al. CrystEngComm 21, 1992-2001 (2019).
84. Ioka, et al. J Chem Phys 154, 234901 (2021).
85. Everts, et al. Sci Rep 8, 14119 (2018).
86. Salis,et al. Langmuir 27, 11597-11604 (2011).
87. Wennerströma, et al. PNAS 117, 10113-10121 (2020).
88. Salgin, et al. Int. J. Electrochem. Sci. 7, 12404-12414 (2012).
89. Dukhin, et al. Colloids Surf B Biointerfaces 121, 257-263 (2014).
90. Kung, et al. Soft Matter 6, 331-341 (2010).
91. Bishop, et al. J Phys Chem Lett 4, 1507-1511 (2013).
92. Schmittschmitt, J et al. Protein Science 12, 2374-2378 (2003).
93. Shaw, et al. Protein Science 10, 1206-1215 (2001).
94. Jiang, et al. J. Agric. Food Chem. 58, 8035-8042 (2010).
95. Shaw, et al. Protein science 10, 1206-1215 (2001).
96. Alberts, B. et al. Molecular Biology of the Cell. (Garland Science, New York, 2002).
97. Nadendla, et al. J. Am. Chem. Soc 139, 17861-17869 (2017).
98. Sun, et al. et al. ACS Omega 3, 4384-4391 (2018).
99. Perini, G. Predictive design of filtration processes in the pharmaceutical industry—the impact of crystal size and shape. (The University of Manchester, Manchester, 2020).
100. Gallardo, et al. HUM GENE THER. 32, 959-974 (2021).
101. Rieser, et al. Pharmaceutics 13, 748 (2021).
102. Hyde, et al. Org Process Res Dev 21, 1355-1370 (2017).
103. Grover, et al. Chem Rev 105, 1-10 (2005).
104. Chernov et al. J Struct Biol 142, 3-21 (2003).
1. Gordon, et al. The Chemist's Companion: A Handbook of Practical Data, Techniques and References. (John Wiley & Sons, New York, 1972).
2. Bharmoria, et al. Journal of Physical Chemistry B 116, 11712-11719 (2012).
3. Mitchell, et al. et al. Acta Crystallogr Sect F Struct Biol Cryst Commun 65, 715-718 (2009).
4. DiMattia, et al. et al. Acta Crystallogr. Sect. F Struct. Biol. Cryst. Commun. 61, 917-921 (2005).
5. Nesse, W. D. Introduction to Optical Minerology. (Oxford University Press, New York, NY, 2004).
6. Bloss, F. D. Crystallography and Crystal Chemistry. (Hold, Rinehart, Winston,Inc., New York, NY, 2012).
7. Polowsky, et al. J Dairy Sci 101, 7714-7723 (2018).
8. Gallardo, et al. Hum Gene Ther 2, 17-18 (2019).

We claim:

1. A method of purifying full capsids of recombinant adeno-associated virus vectors (rAAVs) containing recombinant therapeutic genes from a mixture of full rAAV capsids and empty rAAV capsids having no recombinant therapeutic genes, comprising
(i) mixing the mixture with a crystallization solution, wherein the crystallization solution comprising a pre-determined set of conditions of precipitant concentration, salt concentration, and pH, wherein the pre-determined set of conditions of precipitant concentration, salt concentration, and pH preferentially crystalize the full rAAV capsids over the empty rAAV capsids;
(ii) providing an effective amount of time to allow crystals of the full rAAV capsids to grow; and
(iii) removing the crystals of the full rAAV capsids from the mixture, thereby purifying the full rAAV capsids from the mixture.

2. The method of claim 1, wherein the initial capsid concentration of the mixture of full rAAV capsids and the empty rAAV capsids is between about 0.1×10¹⁴ and about 5×10¹⁴ vg/ml, inclusive, or between about 0.25×10¹⁴ and about 2.5×10¹⁴ vg/ml, inclusive.

3. The method of claim 1, wherein the mixture of full rAAV capsids and empty rAAV capsids comprises about 50%, 60%, 70%, 80%, 90%, or more than 90% full rAAV capsids out of the total rAAV capsids.

4. The method of claim 1, wherein prior to step (i) the method comprises a step of obtaining the crystallization region to provide a phase diagram for each of the full rAAV capsids and the empty rAAV capsids by varying one or more parameters of precipitant concentration, salt concentration, and pH; and a further step of superimposing the phase diagram for the full rAAV capsids and the phase diagram for the empty rAAV capsids to identify the range of precipitant concentration, salt concentration, and pH that preferentially crystalizes the full rAAV capsids over empty rAAV capsids to provide the pre-determined set of conditions.

5. The method of claim 1, wherein: (a) the precipitant is polyethylene glycol (PEG), optionally, wherein the precipitant is PEG8000 or PEG6000, optionally, the precipitant concentration is between about 0.5 and about 12 w/v %, inclusive; (b) salt is sodium chloride or magnesium chloride, optionally, wherein the salt concentration is between about 0.01 to 4.5 M, inclusive; and/or (c) the pH range is between about 5.5 and about 7.5, inclusive.

6. The method of claim 1, wherein the rAAVs are of a serotype selected from the group consisting of AAV1, AAV2, AAV3, AAV 3B, AAV4, AAV5, AAV6, AAV7, AAV8, AAV9, AVV10, AAV11, AAV12, BAAV, AAAV, or AAV VR-942.

7. The method of claim 6, wherein:
(a) the rAAVs are of serotype AAV5, and wherein the pre-determined set of conditions comprise PEG6000 as the precipitant with a concentration between about 1.8 and about 8.0 w/v %, inclusive; sodium chloride as the salt with a concentration between about 0.01 and about 2.2 M, inclusive; and a pH between about 5.5 and about 7.4, inclusive;
(b) the rAAVs are of serotype AAV9, and wherein the pre-determined set of conditions comprise PEG8000 as the precipitant with a concentration between about 2.0 and about 3.5 w/v %, inclusive; sodium chloride as the salt with a concentration between about 0.01 and about 2.3 M, inclusive; and a pH between about 5.5 and about 7.4, inclusive.

8. The method of claim 1, wherein: (a) the crystals are stored at between about 2° C. and about 20° C., inclusive, preferably between about 4° C. and about 10° C., inclusive; and/or (b) the method comprises adding one or more cryoprotectant to the crystals of the full rAAVs capsids and then storing the crystals at between about −1° C. and about −80° C., inclusive, preferably between about −10° C. and about −30° C., inclusive.

9. A crystal of full capsids of recombinant adeno-associated virus vectors (rAAVs) containing recombinant therapeutic genes, wherein the crystal is prepared and purified according to the method of claim 1.

10. The crystal of claim 9, wherein the crystal has minimal empty rAAVs capsids having no recombinant therapeutic genes, about less than 5%, 4%, 3%, 2%, or 1% out of total rAAVs capsids in the crystal.

11. The crystal of full capsids of recombinant adeno-associated virus vectors (rAAVs) of claim 10 comprising serotype AAV5, rAAV8 or rAAV9, containing recombinant therapeutic genes, wherein the crystal is prepared by the steps of
(a) mixing:
(i) a mixture containing full rAAV5 capsids and empty rAAV5 capsids having no recombinant therapeutic genes with one or more crystallization solution, wherein the crystallization solution comprises PEG8000 as a precipitant with a concentration between about 1.8 and about 8.0 w/v %, inclusive; sodium chloride as the salt with a concentration between about 00.01 and about 2.2 M; and a pH between about 5.5 and about 7.4, inclusive;
(ii) a mixture containing full rAAV8 capsids and empty rAAV8 capsids having no recombinant therapeutic genes with one or more crystallization solution, wherein the crystallization solution comprises PEG as a precipitant with a concentration between about 2.0 and about 4.0 w/v %, inclusive; sodium chloride as the salt with a concentration between about 0.01 and about 1.5 M; and a pH between about 5.5 and about 7.2, inclusive; or
(iii) a mixture containing full rAAV5 capsids and empty rAAV9 capsids having no recombinant therapeutic genes with one or more crystallization solution, wherein the crystallization solution comprises PEG8000 as the precipitant with a concentration between about 2.0 and about 7.8 w/v %, inclusive; sodium chloride as the salt with a concentration between about 0.01 and about 2.3 M, inclusive; and a pH between about 5.5 and about 7.4, inclusive; and
(b) providing an effective amount time to allow crystals of the full rAAV5 capsids to grow.

12. The crystal of claim 11, wherein:
(a) for rAAV5, the crystallization solution comprises PEG as the precipitant with a concentration between about 2.0 and about 2.6 w/v %, inclusive; sodium chloride as the salt with a concentration between about 0.01 and about 1.2 M; and a pH between about 5.6 and about 6.9, inclusive; and
(b) for rAAV9, the crystallization solution comprises PEG6000 as the precipitant with a concentration between about 2.5 and about 5.4 w/v %, inclusive; sodium chloride as the salt with a concentration between about 0.7 and about 1.9 M, inclusive; and a pH between about 5.6 and about 6.9, inclusive.

13. The crystal of claim 12, wherein for rAAV5, the crystallization solution comprisesPEG8000 as the precipitant with a concentration between about 2.6 and about 5.6 w/v %, inclusive; magnesium chloride as the salt with a concentration between about 0.3 and about 1.6 M, inclusive; and a pH between about 5.6 and about 6.9, inclusive.

14. The crystal of claim 12, wherein for rAAV9, the crystallization solution comprises PEG8000 as the precipitant with a concentration between about 3.5 and about 4.7 w/v %, inclusive; sodium chloride as the salt with a concentration between about 0.2 and about 1.5 M, inclusive; and a pH between about 5.6 and about 6.9, inclusive.

15. The method of claim 12, wherein the rAAVs are of serotype AAV8, and wherein the pre-determined set of conditions comprise PEG8000 as the precipitant with a concentration between about 3 and about 5 w/v %, inclusive; sodium chloride as the salt with a concentration between about 1.3 and about 2.1 M, inclusive; and a pH between about 5.6 and about 6.9, inclusive.

16. The method of claim 12, wherein the rAAVs are of serotype AAV5, and wherein the pre-determined set of conditions comprise PEG6000 as the precipitant with a concentration between about 1.5 and about 8 w/v %, inclusive; sodium chloride as the salt with a concentration between about 0.01 and about 2 M, inclusive; and a pH between about 5.5 and about 7.4, inclusive.

17. The method of claim 12, wherein the rAAVs are of serotype AAV5, and wherein the pre-determined set of conditions comprise PEG6000 as the precipitant with a concentration between about 2.1 and about 3.5 w/v %, inclusive; sodium chloride as the salt with a concentration between about 0.01 and about 0.8 M, inclusive; and a pH between about 5.6 and about 6.9, inclusive.

18. The method of claim 12, wherein the rAAVs are of serotype AAV9, and wherein the pre-determined set of conditions comprise PEG8000 as the precipitant with a concentration between about 1.8 and about 7.5 w/v %, inclusive; sodium chloride as the salt with a concentration between about 0.01 and about 2.3 M, inclusive; and a pH between about 5.5 and about 7.4, inclusive.

19. The method of claim 1, wherein the method comprises reconstituting the crystals of the full rAAVs capsids with a pharmaceutically acceptable excipient for administering to a subject in need of gene therapies.

20. The method of claim 19, wherein the subject is in need of a genetic enzyme replacement therapy or has one or more of Duchenne muscular dystrophy, limb girdle muscular dystrophy type 2D, Leber's hereditary optic neuropathy, late infantile neuronal ceroid lipofuscinosis, rheumatoid arthritis, mucopolysaccharidosis, spinal muscular atrophy, X-linked juvenile retinoschisis, Dysferlin deficiency, hemophilia A, hemophilia B, metachromatic leukodystrophy, idiopathic Parkinson's disease, and Alzheimer's disease.
